(19) United States
(12) Patent Application Publication
(10) Pub. No.: US 2022/0041719 A1
(43) Pub. Date: Feb. 10, 2022

(54) MULTISPECIFIC ANTIGEN-BINDING MOLECULES

(71) Applicant: MORPHOSYS AG, Planegg (DE)

(72) Inventors: Andreas Bültmann, Planegg (DE); Karin Felderer, Munich (DE); Sebastian Jäger, Gräfelfing (DE); Steffen Runz, Heidelberg (DE); Johannes Urban, Munich (DE)

(21) Appl. No.: 17/297,735

(22) PCT Filed: Dec. 4, 2019
(86) PCT No.: PCT/EP2019/083638

§ 371 Date: May 27, 2021

(30) Foreign Application Priority Data
Dec. 5, 2018 (EP) 18210328.2

(51) Int. Cl. C07K 16/28; A61P 35/00

(52) U.S. Cl. CPC C07K 16/2809; A61P 35/00; C07K 2317/52; C07K 2317/31; C07K 2317/56; C07K 2317/565; C07K 2317/35; C07K 2317/55

(57) ABSTRACT

Novel antigen-binding molecules are provided, with the ability to target different antigens with different valency, e.g. one antigen monovalently and another antigen bivalently.

### TECHNICAL FIELD

The present disclosure provides novel antigen-binding molecules capable of simultaneously binding to at least two different antigens. The ability to target two different antigens with different valency (e.g. one antigen monovalently and one antigen bivalently) is a particular useful aspect of the antigen-binding molecules disclosed herein. The novel molecules described herein preferably utilize heterodimeric Fc regions of an immunoglobulin. Methods of producing and using such novel antigen-binding molecules and compositions comprising such, particularly for therapeutic purposes are also described.

### BACKGROUND

Multispecific antigen-binding molecules, such as bispecific antibodies, capable of binding to two or more antigens are of great interest for therapeutic applications, as they allow for the simultaneous binding and inactivation of two or more target antigens, and as such represent an alternative approach to conventional combination therapies. However, for many antigens that are attractive as co-targets for such multispecific formats, the preferred binding to at least one antigen is monovalent rather than bivalent.

Bivalent binding of regular immunoglobulin antibodies has been found to crosslink certain cell surface receptors and thereby mimic the effect of the natural ligand. Cross-linking can lead to receptor activation (e.g. receptor phosphorylation). In contrast, monovalent binding (such as of Fabs derived from the same antibody) does not lead to receptor cross-linking and, if the appropriate epitope is targeted, prevent the natural ligand from binding. Thus, while bivalent antigen-binding might result in an agonistic activity, monovalent binding to the same antigen might result in an antagonistic activity. Examples of such receptors are the insulin receptor (Kahn et al., Proc Natl Acad Sci USA. (1978) 75:4209-13), the EGF receptor (Schreiber et al., J Biol Chem. (1983) 258:846-53), the EPO receptor (Schneider et al, Blood (1997) 89:473-82), the GH receptor (Wan et al., Mol Endocrinol. (2003) 17:2240-50) or the beta2-Adrenoceptor (Mijares et al., Mol Pharmacol. (2000) 58:373-9).

Other exemplary antigens for which it may be therapeutically beneficial or necessary to co-engage monovalently include members of the T-cell receptor complex, such as CD3, the low affinity Fc gamma receptors (FcyRs), toll-like receptors (TLRs), cytokines, chemokines, cytokine receptors, chemokine receptors or receptor-tyrosine kinases (RTKs).

A large number of multispecific antigen-binding formats were developed in the recent years, including tetravalent IgG-single-chain variable fragment (scFv) fusions (see e.g. Coloma & Morrison, Nat Biotechnol 15, 159-163 (1997)), tetravalent IgG-like dual-variable domain (DVD) antibodies (Wu et al., Nat Biotechnol 25, 1290-1297 (2007)), or bivalent rat/mouse hybrid bispecific IgGs (see e.g. Lindhofer et al., J Immunol 155, 219-225 (1995)).

However, a disadvantage of such IgG based approaches is that they bind to the co-targeted antigen in a multivalent (e.g. bivalent) fashion, thus leading to a potential non-specific activation and associated side-effects. The production of these IgG-based multispecific constructs is also a major hurdle, as the homodimerization of antibody heavy chains and/or the mispairing of antibody heavy and light chains of different specificities upon co-expression decreases the yield of the correctly assembled construct and results in a number of non-functional side products from which the desired construct may be difficult to separate.

On the other hand, several multispecific antigen-binding formats, wherein an antibody core structure (IgA, IgD, IgE, IgG or IgM) is no longer maintained were developed. Examples include diabodies (see e.g. Holliger et al., Proc Natl Acad Sci USA 90, 6444-6448 (1995)), tandem scFv molecules (see e.g. Bargou et al., Science 321, 974-977 (2008)), and various derivatives thereof. However, antigen-binding formats lacking the IgG core structure and being capable of providing monovalent binding to at least one antigen are often disadvantageous because of their poor biophysical and pharmacokinetic properties, such as short half-life and their incapability to mediate effector function (such as ADCC or CDC).

WO 2016/086189 discloses various multispecific bi- and trivalent antibody formats based on a full IgG molecule wherein either one Fab arm of the IgG molecule is replaced by a single-chain Fv domain (“bottle opener” format) or wherein an additional Fv or single-chain Fv domain is fused to the C-terminus of one or both heavy chains of the IgG molecule. One additional theoretical described antibody format refers to the “central-Fv” format which incorporates an additional Fv domain between the two Fab arms and the Fc region of an IgG in order to form a third antigen binding site. However, the application neither provides experimental evidence that the described construct does actually function nor does it suggests to combine the specific Fc modifications utilized in the antigen-binding molecules of the present disclosure, responsible for efficient heterodimerization of the two Fc region subunits and/or abolished effector function.

The presently disclosed novel antigen-binding molecules solve the aforementioned shortcomings of the IgG- and the non-IgG based antigen-binding molecules by introducing a format that allows for the simultaneous bivalent and monovalent co-engagement of different antigens with all the desirable properties provided by a regular full-length immunoglobulin and which are easy to purify from the culture supernatant of respective production cell lines.

### SUMMARY

The present disclosure pertains to novel multispecific antigen-binding molecules, which allow for monovalent binding to at least one antigen, whilst retaining the properties of a regular immunoglobulin molecule in respect of size and presence of an Fc region.

In an embodiment, an antigen-binding molecule according to the present disclosure allows for trivalent binding to one antigen. In such an embodiment, the antigen-binding molecule comprises at least three Fv regions, wherein each Fv region binds to the same antigen. In such an embodiment, the antigen-binding molecule according to the present disclosure refers to a trivalent monospecific antigen-binding molecule.

In an embodiment, an antigen-binding molecule according to the present disclosure allows for a monovalent binding to three different antigens. In such an embodiment, the antigen-binding molecule comprises at least three Fv regions, wherein each of the three Fv regions binds to one different antigen. In such an embodiment, the antigen-binding molecule according to the present disclosure refers to a trivalent trispecific antigen-binding molecule.

In a preferred embodiment, an antigen-binding molecule according to the present disclosure allows for a bivalent binding to one antigen and monovalent binding to a second antigen. In such an embodiment, the antigen-binding molecule comprises at least three Fv regions, wherein two of the three Fv regions binds to one of the two target antigens and the third Fv region binds to the other target antigen. In such a preferred embodiment, an antigen-binding molecule according to the present disclosure refers to a trivalent bispecific antigen-binding molecule.

Accordingly, in an embodiment, the present disclosure provides an antigen-binding molecule comprising
a) a first Fab comprising a first Fv region, which specifically binds to a first antigen, b) a second Fv region which specifically binds to a second antigen and
c) a second Fab comprising a third Fv region, which specifically binds to a third antigen, and
d) a Fc region composed of a first and second Fc region subunit; wherein
   I. the C-terminus of the heavy or light chain of the first Fab is fused to the N-terminus of the VH or VL of the second Fv region, and wherein
   II. the C-terminus of the VH or VL of the second Fv region is fused to the N-terminus of the first Fc region subunit and the N-terminus of the second Fc domain subunit is fused to the C-terminus of the complementary variable domain of the second Fv region, and wherein
   III. the C-terminus of the heavy or light chain of the second Fab is fused to the N-terminus of the VH or VL of the second Fv region with the proviso that the first and second Fab are fused to distinct variable domains of the second Fv region, and wherein
   IV. in the CH3 domain of first Fc region subunit, the threonine residue at position 366 is replaced with a tryptophan residue (T366W) and the serine residue at position 354 is replaced with a cysteine residue (S354C) and in the CH3 domain of the second Fc region subunit the tyrosine residue at position 407 is replaced with a valine residue (Y407V), the threonine residue at position 366 is replaced with a serine residue (T366S), the leucine residue at position 368 is replaced with an alanine residue (L368A) and the tyrosine residue at position 349 is replaced by a cysteine residue (Y349C) with numbering according EU index.

In an embodiment, the third antigen is identical to the first or the second antigen. In an embodiment, the third antigen is identical to the first antigen. In an embodiment, the first Fab is identical to the second Fab.

In an embodiment, the second Fab is fused to the second Fv region. In an embodiment, the C-terminus of the second Fab is fused to the N-terminus of the second Fv region. In an embodiment, the C-terminus of the heavy or light chain of the second Fab is fused to the N-terminus of the VH or VL of the second Fv region with the proviso that the first and second Fab are fused to distinct variable domains of the second Fv region. In an embodiment, the C-terminus of the CH1 or CL of the second Fab is fused to the N-terminus of the VH or VL of the second Fv region with the proviso that the first and second Fab are fused to distinct variable domains of the second Fv region.

In an embodiment, each fusion occurs via a linker. In an embodiment, each fusion occurs via a peptide linker. In an embodiment, the peptide linkers are identical or different. In an embodiment, each fusion occurs via a peptide linker each having a length of at least 1 amino acids residue. In an embodiment, each fusion occurs via a peptide linker each having a length of at least 5 amino acids residues. In an embodiment, the peptide linkers are of identical length. In an embodiment, the peptide linkers are of different length. In an embodiment, the first Fab is fused to the second Fv region via a peptide linker.

In an embodiment, the C-terminus of the heavy or light chain of the first Fab is fused to the N-terminus of the VH or VL of the second Fv region via a peptide linker. In an embodiment, the C-terminus of the CH1 or CL of the first Fab is fused to the N-terminus of the VH or VL of the second Fv region via a peptide linker.

In an embodiment, the second Fv region is fused to the Fc region via peptide linkers. In an embodiment, the second Fv region is fused to the Fc region via two peptide linkers. In an embodiment, the C-terminus of the VH or VL of the second Fv region is fused to the N-terminus of the first Fc region subunit via a first peptide linker and the N-terminus of the second Fc region subunit is fused to the C-terminus of the complementary variable domain of the second Fv region via a second peptide linker. In an embodiment, the first and second peptide linker are different. In an embodiment, the first and second peptide linker are identical.

In an embodiment, the first and second peptide linker are linked via one more interchain disulfide bridges. In an embodiment, the first and second peptide linker comprises one or more cysteine residues allowing for the formation of one or more interchain disulfide bridges between the first and second peptide linker. In an embodiment, the first and second peptide linker comprises one or more cysteine residues allowing for the formation of one or more interchain disulfide bridges between the first and second peptide linker resulting in a disulfide bridge stabilized dimeric peptide linker. In an embodiment, the first and second peptide linker is derived from an immunoglobulin hinge, preferably from an IgG hinge, preferably from a human IgG hinge, preferably a human IgG1 hinge of fragment thereof. In an embodiment, the fusion between the VH or VL of the second Fv region and the first or second Fc region subunit occurs via a peptide linker comprising an IgG hinge or a portion or fragment thereof. In an embodiment, the IgG hinge is a human IgG hinge. In an embodiment, the human IgG hinge is a human IgG1 hinge. In an embodiment, the human IgG1 hinge comprises the amino sequence DKTHTCPPCP (SEQ ID NO: 13). In an embodiment, the fusion between the second Fv region and the Fc region occurs via an IgG hinge region or part thereof.

In an embodiment, the first Fab, the second Fv region and the Fc region are fused to each of their fusion partners via a peptide linker. In an embodiment, the peptide linkers are identical or different. In an embodiment, the peptide linkers are identical. In an embodiment, the peptide linkers are different. In an embodiment, each of the peptide linkers has a length of at least 1 amino acid residue. In an embodiment, each of the peptide linkers has a length of at least 5 amino acid residues. In an embodiment, each of the peptide linkers has a length of between 1 and 70 amino acid residues. In an embodiment, the peptide linkers are of identical length. In an embodiment, the peptide linkers are of different length.

In an embodiment, the peptide linker is selected from the group consisting of but not limited to QPKAAP (SEQ ID NO: 12), ASTKGP (SEQ ID NO: 11), (G₄S)₃ (SEQ ID NO: 33), (GGS)₃ (SEQ ID NO: 10), DKTHTCPPCP (SEQ ID NO: 13), QPKAAPDKTHTCPPCP (SEQ ID NO: 15), and ASTKGPDKTHTCPPCP (SEQ ID NO: 14).

In an embodiment, the VH and the VL of the second Fv region are optionally linked via an interchain disulfide bridge. In an embodiment, the VH and the VL of the second Fv region are linked via an interchain disulfide bridge.

In an embodiment, the disulfide bridge is introduced between the following positions with numbering according Kabat:
a. VH position 44 and VL position 100, and/or
b. VH position 105 and VL position 43 and/or
c. VH position 101 and VL position 100

In an embodiment, the disulfide bridge is introduced between the positions with numbering according Kabat: VH position 44 and VL position 100. In an embodiment, the disulfide bridge is introduced between the positions with numbering according Kabat: VH position 105 and VL position 43. In an embodiment, the disulfide bridge is introduced between the positions with numbering according Kabat: VH position 101 and VL position 100. In an embodiment, the second Fab is fused to the second Fv region via a peptide linker

In an embodiment, the C-terminus of the heavy or light chain of the second Fab is fused to the N-terminus of the VH or VL of the second Fv region via a peptide linker. In an embodiment, the C-terminus of the CH1 or CL of the second Fab is fused to the N-terminus of the VH or VL of the second Fv region via a peptide linker. In an embodiment, the C-terminus of the CH1 or CL of the second Fab is fused to the N-terminus of the VH or VL of the second Fv region with the proviso that first and second Fab are fused to distinct variable domains of the second Fv region and wherein each fusion occurs via a peptide linker.

In an embodiment, the antigen-binding molecule according to the present disclosure is composed of at least 4 polypeptides. In an embodiment, an antigen-binding molecule according to the present disclosure is composed of at least 4 polypeptides, wherein
a. a first polypeptide comprises the light or heavy chain of the first Fab,
b, a second polypeptide comprises from its N-terminus to its C-terminus
   i. the complementary light or heavy chain of the first Fab,
   ii. the VH or VL of the second Fv region and
   iii. the first or second Fc domain subunit
c. a third polypeptide comprises from its N-terminus to its C-terminus
   i. the light or heavy chain of the second Fab,
   ii. the complementary VH or VL of the second Fv region and
   iii. the complementary first or second Fc domain subunit.
d. a fourth polypeptide comprises the complementary light or heavy chain of the second Fab.

In an embodiment, the first polypeptide is identical to the fourth polypeptide. In an embodiment, the light chain of the first Fab is identical to light chain of the second Fab. In an embodiment, the heavy chain of the first Fab is identical to the heavy chain of the second Fab. In an embodiment, the heavy and light chain of the first and second Fab are identical. In an embodiment, an antigen-binding molecule according to the present disclosure has a structure as depicted in FIG. 1B.

In an embodiment, an antigen-binding molecule according to the present disclosure provides monovalent binding to the first, second antigen or third antigen. In an embodiment, an antigen-binding molecule according to the present disclosure provides monovalent binding to the first antigen. In an embodiment, an antigen-binding molecule according to the present disclosure provides monovalent binding to the second antigen. In an embodiment, an antigen-binding molecule according to the present disclosure provides monovalent binding to the third antigen. In an embodiment, the third antigen is identical to the first or the second antigen. In an embodiment, the third antigen is identical to the first antigen. In an embodiment, an antigen-binding molecule according to the present disclosure provides monovalent binding to the first antigen and bivalent binding to the second antigen. In an embodiment, an antigen-binding molecule according to the present disclosure provides bivalent binding to the first antigen and monovalent binding to the second antigen.

In an embodiment, an antigen-binding molecule according to the present disclosure is a bispecific antigen-binding molecule. In an embodiment, an antigen-binding molecule according to the present disclosure is a trivalent bispecific antigen-binding molecule.

In an embodiment, the first or second antigen is a member of the T-cell receptor complex. In an embodiment, the second antigen is a member of the T-cell receptor complex. In an embodiment, the first antigen is a member of the T-cell receptor complex. In an embodiment, the member of the T-cell receptor complex is CD3. In an embodiment, the first antigen is CD3. In an embodiment, the second antigen is CD3.

In an embodiment, an antigen-binding molecule according to the present disclosure provides bivalent binding to the first antigen and monovalent binding to the second antigen, wherein the second antigen is CD3. In an embodiment, an antigen-binding molecule according to the present disclosure provides monovalent binding to the first antigen and bivalent binding to the second antigen, wherein the first antigen is CD3.

In an embodiment, the present disclosure provides an antigen-binding molecule, wherein the Fc region comprises one or more amino acid modifications promoting the association of the first and second Fc region subunit. In an embodiment, the present disclosure provides an antigen-binding molecule, wherein each Fc region subunit comprises one or more amino acid modifications promoting the association of the first and second Fc region subunit. In an embodiment, each Fc region subunit comprises a different amino acid modification, such that the heterodimeric Fc region is more stable than the homodimeric Fc region. In an embodiment, each Fc region subunit comprises a different amino acid modification, such that the association of the first and second Fc region subunit is promoted. In an embodiment, the Fc region is an immunoglobulin Fc region. In an embodiment, the immunoglobulin Fc region is an IgG Fc region. In an embodiment, the IgG Fc region is a human IgG Fc region. In an embodiment, the human IgG Fc region is a human IgG1 region.

In an embodiment, in the CH3 domain of the first Fc region subunit the threonine residue at position 366 is replaced with a tryptophan residue (T366W) and in the CH3 domain of the second Fc region subunit the tyrosine residue at position 407 is replaced with a valine residue (Y407V) with numbering according EU index. In an embodiment, in the second Fc region subunit, the threonine residue at position 366 is replaced with a serine residue (T366S) and the leucine residue at position 368 is replaced with an alanine residue (L368A) with numbering according EU index. In an embodiment, in the first Fc region subunit the serine residue at position 354 is replaced with a cysteine residue (S354C), and in the second Fc region subunit of the Fc region the tyrosine residue at position 349 is replaced by a cysteine residue (Y349C) with numbering according EU index.

In an embodiment, in the CH3 domain of first Fc region subunit, the threonine residue at position 366 is replaced with a tryptophan residue (T366W) and the serine residue at position 354 is replaced with a cysteine residue (S354C) and in the CH3 domain of the second Fc region subunit the tyrosine residue at position 407 is replaced with a valine residue (Y407V), the threonine residue at position 366 is replaced with a serine residue (T366S), the leucine residue at position 368 is replaced with an alanine residue (L368A) and the tyrosine residue at position 349 is replaced by a cysteine residue (Y349C) with numbering according EU index.

In an embodiment, the Fc region is engineered to have an altered binding affinity to an Fc receptor and/or to C1q and/or to have an altered effector function when compared to the non-engineered Fc region. In an embodiment, the engineered Fc region has a higher binding affinity to an Fc receptor and/or to C1q and/or has increased effector function when compared to the non-engineered Fc region.

In an embodiment, the engineered Fc region has a lower binding affinity to an Fc receptor and/or to C1q and/or has reduced effector function when compared to the non-engineered Fc region. In an embodiment, the engineered Fc region has substantially no binding affinity to an Fc receptor and/or to C1q and/or has substantially no effector function when compared to the non-engineered Fc region. In an embodiment, the engineered Fc region has no binding affinity to an Fc receptor and/or to C1q and/or has no effector function when compared to the non-engineered Fc region.

In an embodiment, the present disclosure provides an antigen-binding molecule, wherein in each Fc region subunit at least one of the 5 amino acid residues in the positions corresponding to positions L234, L235, G237, A330, P331 with numbering according EU index in a human IgG1 are mutated. In an embodiment, the present disclosure provides an antigen-binding molecule, wherein in each Fc region subunit at least one of the 5 amino acid residues in the positions corresponding to positions L234, L235, G237, A330, P331 with numbering according EU index in a human IgG1 are mutated and wherein the engineered Fc region has substantially no binding affinity to an Fc receptor and/or to C1q and/or has substantially no effector function when compared to the non-engineered Fc region. In an embodiment, the present disclosure provides an antigen-binding molecule, wherein in each Fc region subunit at least one of the 5 amino acid residues in the positions corresponding to positions L234, L235, G237, A330, P331 with numbering according EU index in a human IgG1 are mutated to A, E, A, S, and S, respectively. In an embodiment, the present disclosure provides an antigen-binding molecule, wherein in each Fc region subunit at least one of the 5 amino acid residues in the positions corresponding to positions L234, L235, G237, A330, P331 with numbering according EU index in a human IgG1 are mutated to A, E, A, S, and S, respectively and wherein the engineered Fc region has substantially no binding affinity to an Fc receptor and/or to C1q and/or has substantially no effector function when compared to the non-engineered Fc region.

In an embodiment, the present disclosure provides an antigen-binding molecule, wherein in the Fc region subunit at least 5 amino acid residues in the positions corresponding to positions L234, L235, G237, A330, P331 with numbering according EU index in a human IgG1 are mutated to A, E, A, S, and S, respectively. In an embodiment, the present disclosure provides an antigen-binding molecule, wherein in the Fc region subunit at least 5 amino acid residues in the positions corresponding to positions L234, L235, G237, A330, P331 with numbering according EU index in a human IgG1 are mutated to A, E, A, S, and S, respectively and wherein the engineered Fc region has substantially no binding affinity to an Fc receptor and/or to C1q and/or has substantially no effector function when compared to the non-engineered Fc region.

In an embodiment, the antigen-binding molecule according to the present disclosure is a polyclonal or monoclonal antigen-binding molecule. In an embodiment, the antigen-binding molecule according to the present disclosure is a monoclonal antigen-binding molecule.

In an embodiment, the antigen-binding molecule according to the present disclosure is an isolated antigen-binding molecule. In an embodiment, the antigen-binding molecule according to the present disclosure is a recombinant antigen-binding molecule. In an embodiment, the antigen-binding molecule according to the present disclosure is an isolated recombinant antigen-binding molecule.

In an embodiment, the present disclosure provides a nucleic acid composition comprising a nucleic acid sequence or a plurality of nucleic acid sequences encoding an antigen-binding molecule according to the present disclosure. In an embodiment, an antigen-binding molecule according to the present disclosure is encoded by a nucleic acid composition according to the present disclosure. In an embodiment, the present disclosure provides a vector composition comprising a vector or a plurality of vectors comprising the nucleic acid composition according to the present disclosure. In an embodiment, the present disclosure provides to a host cell comprising a vector composition according to the present disclosure or a nucleic acid composition according to the present disclosure encoding an antigen-binding molecule according to the present disclosure. In an embodiment, the present disclosure provides a host cell comprising a nucleic acid composition according to the present disclosure or the vector composition according to the present disclosure. In an embodiment, the present disclosure provides a host cell, wherein the host cell is a eukaryotic cell, particularly a mammalian cell. In an embodiment, the present disclosure provides a host cell, wherein the host cell is a eukaryotic cell. In an embodiment, the present disclosure provides a host cell, wherein the host cell is a mammalian cell.

In an embodiment, the present disclosure provides a method of producing an antigen-binding molecule according to the present disclosure, comprising the steps of a) culturing a host cell according to the present disclosure under conditions suitable for the expression of the antigen-binding molecule and b) recovering the antigen-binding molecule. In an embodiment, the present disclosure provides an antigen-binding molecule produced by the method disclosed herein.

In an embodiment, the present disclosure provides a pharmaceutical composition comprising an antigen-binding molecule according to the present disclosure and a pharmaceutically acceptable carrier. In an embodiment, the present disclosure provides a pharmaceutical composition comprising an antigen-binding molecule according to the present disclosure for use as a medicament. In an embodiment, the present disclosure provides an antigen-binding molecule according to the present disclosure or a pharmaceutical composition according to the present disclosure for use in the treatment of a disease. In an embodiment, the present disclosure provides an antigen-binding molecule according to the present disclosure or a pharmaceutical composition according to the present disclosure for use in the treatment of a disease in an individual in need thereof. In an embodiment, the present disclosure provides the use of an antigen-binding molecule according to the present disclosure for the manufacture of a medicament. In an embodiment, the present disclosure provides the use of an antigen-binding molecule according to the present disclosure for the manufacture of a medicament for the treatment of a disease in an individual in need thereof. In an embodiment, the present disclosure pertains to a method of treating a disease in an individual, comprising administering to said individual a therapeutically effective amount of a pharmaceutical composition comprising an antigen-binding molecule according to the present disclosure.

### BRIEF DESCRIPTION OF THE DRAWINGS

FIG. 1: Design of a bivalent or trivalent bispecific antigen-binding molecule according to the present disclosure comprising two or three Fv regions.

FIG. 1A: Structure of a bivalent bispecific antigen-binding molecule according to the present disclosure. The structure comprises one Fab arm (Fab₁) from a regular immunoglobulin comprising a first binding site formed by a first Fv region (Fv₁). A second antigen-binding site is formed by a second Fv region (Fv₂). Each variable domain (VH₂ and VL₂) of the second Fv region (Fv₂) is fused via a peptide linker to the C-terminus of one CH2 domain of an Fc region subunit. The two peptide linkers may include interchain-cysteines which allows for the formation of stabilizing disulfide bridges between the two linkers (bold cross strokes). Additional peptide linkers fuse the N-terminus of the VH or VL (VH₂ and VL₂) of the second Fv region (Fv₂) with the CH1 or CL of first Fab. Heterodimerization of the polypeptide chains comprising the two Fc region subunits is promoted by modification of the CH3 domains in each Fc region subunit by approaches of disulfide stabilization and knob-into-holes technology. In addition, disulfide stabilization in the VH₂/VL₂ interface of the second Fv region may be applied (not indicated). The CH2 domain in each Fc region subunit may be additionally modified in order to enhance or to abolish Fc mediated effector function (not indicated).

FIG. 1B: Structure of a trivalent bispecific antigen-binding molecule according to the present disclosure comprising a first Fab (Fab₁), second Fab (Fab)₂ and a second Fv region (Fv₂). The molecule comprises two Fab arms from a regular immunoglobulin comprising a first and third antigen-binding site formed by a first Fv region (Fv₁) and a third Fv region (Fv₃). A second antigen-binding site is formed by a second Fv region (Fv₂). Each variable domain (VH₂ and VL₂) of the second Fv region (Fv₂) is fused via a peptide linker to the C-terminus of one CH2 domain of an Fc region subunit. The peptide linkers include interchain-cysteines which allow for the formation of stabilizing disulfide bridges between the two linkers (bold cross strokes). Additional peptide linkers fuse the N-terminus of the VH₂ and VL₂ of the second Fv region with the N-terminus of the CH1 or CL of the first Fab and second Fab, respectively. Heterodimerization of the polypeptide chains comprising the two Fc region subunits is promoted by modification of the CH3 domains in each Fc region subunit by approaches of disulfide stabilization and knob-into-holes technology. In addition, disulfide stabilization in the VH₂/VL₂ interface of the second Fv region may be applied (not indicated). The CH2 domain in each Fc region subunit may be additionally modified in order to enhance or to abolish Fc mediated effector function (not indicated).

FIG. 2: Design of bivalent or trivalent bispecific antigen-binding molecules according to the present disclosure comprising two or three Fv regions and additional IgG constant domains.

FIG. 2A: Structure of a bivalent bispecific antigen-binding molecule according to the present disclosure. The molecule comprises one Fab arm from a regular immunoglobulin comprising a first binding site formed by a first Fv region (Fv₁). A second antigen-binding site is formed by a second Fv region (Fv₂) of a third Fab (Fab₃). The C-terminus of the heavy and light chain of the third Fab (CH1 or CL, respectively) is fused via peptide linkers to the N-terminus of the CH2 domains of the Fc region. The peptide linkers include interchain-cysteines, which allow for the formation of stabilizing disulfide bridges between the two linkers (bold cross strokes). Additional peptide linkers fuse the N-terminus of the VH or VL of the second Fv region with the C-terminus of the CH1 or CL of the first Fab, respectively. Heterodimerization of the polypeptide chains comprising the two Fc region subunits is promoted by modification of the CH3 domains in each Fc region subunit by approaches of disulfide stabilization and knob-into-holes technology. In addition, disulfide stabilization in the VH₂/VL₂ interface of the second Fv region may be applied (not indicated). The CH2 domains may be additionally modified in order to enhance or to abolish Fc mediated effector function (not indicated).

FIG. 2B: Structure of a trivalent bispecific antigen-binding molecule according to the present disclosure comprising a first Fab (Fab₁), a second Fab (Fab₂) and third Fab (Fab₃). The molecule comprises two Fab arms (Fab₁ and Fab₂) from a regular immunoglobulin comprising a first and third antigen-binding site formed by a first Fv region (Fv₁) and a third Fv region (Fv₃). A second antigen-binding site is formed by a second Fv region (Fv₂) of a third Fab (Fab₃). The C-terminus of the constant domains of the first and third Fab (CH1 or CL, respectively) are fused via peptide linkers to the N-terminus of respective CH2 domains of the Fc region. The peptide linkers include interchain-cysteines which allow for the formation of stabilizing disulfide bridges between the two linkers (bold cross strokes). Additional peptide linkers fuse the N-terminus of the VH or VL of the second Fv region (Fv₂) with the C-terminus of the CH1 or CL of the first Fab and second Fab, respectively. Heterodimerization of the polypeptide chains comprising the two Fc region subunits is promoted by modification of the CH3 domains in each Fc region subunit by approaches of disulfide stabilization and knob-into-holes technology. In addition, disulfide stabilization in the VH/VL interface of the second Fv region may be applied (not indicated). The CH2 domains may be additionally modified in order to enhance or to inhibit Fc mediated effector function (not indicated).

FIG. 3: Cell binding of 5 mammalian produced and purified bispecific trivalent antigen-binding molecules according to the present disclosure with a structure as depicted in FIG. 1B with bivalent binding to HER2 and monovalent binding to CD3 (Constructs 1, 3, 4) and negative control (Construct 5). FIG. 3A shows cell binding (signal over background) to CD3 positive Jurkat cells as a function of Construct concentration determined by flow cytometry. FIG. 3B depicts the same as FIG. 3A with the difference that binding to HER2 positive human adenocarcinoma SKOV-3 cells is shown.

FIG. 4: Evaluation of the functional activity of bispecific trivalent antigen-binding molecules according to the present disclosure with a structure as depicted in FIG. 1B with bivalent binding to HER2 and monovalent binding to CD3 (Constructs 1, 3, 4) and negative control (Construct 5) in a NFAT Reporter Gene Assay using Jurkat cells transiently transfected with the NFAT reporter gene construct used as surrogate effector cells. As target cells either the HER2 positive human adenocarcinoma SKOV-3 or the HER2 negative human adenocarcinoma MDA-MB-468 cell line are used. FIG. 4A is a graph showing the relative fluorescence of SKOV-3 cells as a function of Construct concentration. FIG. 4B is a graph showing the relative fluorescence of MDA-MB-468 cells as a function of Construct concentration.

FIG. 5: Cytotoxicity assay of bispecific trivalent antigen-binding molecules according to the present disclosure with a structure as depicted in FIG. 1B with bivalent binding to HER2 and monovalent binding to CD3 (Constructs 1, 3, 4) or negative control (Construct 5) on either HER2 expressing SKBR3 cells or HER2 negative MDA-MB-468 cells in presence of human derived PBMCs. Cytotoxic activity of PBMCs is assessed by measuring incorporated CellToxGreen fluorescence. The graph showing the relative fluorescence of HER2 expressing SKBR3 cells and HER negative MDA-MV-468 cells as a function of Construct concentration.

### DETAILED DESCRIPTION OF THE INVENTION

The present disclosure pertains to antigen-binding molecules that are suited to co-engage two or more antigens simultaneously.

### Definitions

The terms “comprising”, “comprises” and “comprised of” “as used herein are synonymous with “including”, “includes” or “containing”, “contains”, or “composed of”, and are inclusive or open-ended and do not exclude additional, non-recited members, elements or method steps.

The term “polypeptide” as used herein refer to a polymer of amino acid residues and does not refer to a specific length of a product. The term applies to naturally occurring amino acid polymers and non-naturally occurring amino acid polymers. Unless otherwise indicated, a particular amino acid sequence of a polypeptide also implicitly encompasses conservatively modified variants thereof (e.g. by replacing an amino acid residue with another amino acid residue having similar structural and/or chemical properties). A polypeptide may be derived from a natural biological source or produced by recombinant technology, but is not necessarily translated from a designated nucleic acid sequence. It may be generated in any manner, including chemical synthesis.

The term “antigen-binding molecule” as used herein, refers in its broadest sense to a proteinacious molecule that specifically binds to at least one antigen. An antigen-binding molecule may be composed of one or more polypeptides. Examples of antigen-binding molecules are immunoglobulins and derivatives and/or fragments thereof. Antigen-binding molecules according to the present disclosure are based on a regular immunoglobulin (e.g. IgG) structure that incorporates an additional Fv region between the two Fab arms and the Fc region. The antigen-binding molecule as disclosed herein may also lack one of the two Fabs arms of a regular IgG. In such an embodiment, the additional Fv region is incorporated between one Fab arm and the Fc region of a regular immunoglobulin structure. Other proteinaceous antigen-binding molecules include scaffolds with antibody-like properties, such as affibodies (which comprise the Z-domain of protein A), immunity proteins (such as ImmE7), cytochrome b562, proteins comprising ankyrin repeats, PDZ domains or Kunitz domains, insect defensin A, scorpion toxins (such as charybdotoxin or CTLA-4), knottins (such as Min-23, neocarzinostatin, CBM4-2 or tendamistat), anticalins or armadillo repeat proteins.

The term “antibody” molecule or “immunoglobulin” (Ig) molecule used herein refers to a protein comprising at least two heavy (H) chains and two light (L) chains interconnected by disulfide bonds, which interacts with an antigen. Each heavy chain (HC) is comprised of a heavy chain variable domain (abbreviated herein as VH) and a heavy chain constant region. The heavy chain constant region is comprised of three domains, CH1, CH2 and CH3. Each light chain (LC) is comprised of a light chain variable domain (abbreviated herein as VL) and a light chain constant region. The light chain constant region is comprised of one domain, CL. The VH and VL domains can be further subdivided into regions of hypervariability, termed complementarity determining regions (CDR), interspersed with regions that are more conserved, termed framework regions (FR). Each VH and VL is composed of three CDRs and four FR's arranged from N-terminus to C-terminus in the following order: FR1, CDR1, FR2, CDR2, FR3, CDR3, and FR4. The variable domains of the heavy and light chains (VH and VL) contain a “binding site” or “antigen-binding site” that interacts with an antigen. The constant regions of the antibodies may mediate the binding of the immunoglobulin to host tissues or factors, including various cells of the immune system (e.g., effector cells) and the first component (C1q) of the classical complement system. The term “antibody” includes for example, monoclonal antibodies, human antibodies, humanized antibodies, camelised antibodies and chimeric antibodies. The antibodies can be of any isotype (e.g., IgG, IgE, IgM, IgD, IgA and IgY), class (e.g., IgG1, IgG2, IgG3, IgG4, IgA1 and IgA2) or subclass. Both the light and heavy chains are divided into regions of structural and functional homology.

The term “antibody fragment” as used herein, refers to one or more portions of an antibody that retain the ability to specifically interact with (e.g., by binding, steric hindrance, stabilizing spatial distribution) an antigen. Examples of antibody fragments include, but are not limited to, a Fab, a monovalent fragment consisting of the VL, VH, CL and CH1 domains, wherein the Fab heavy chain (HC) is formed by the VH and CH1 domains (VH-CH1) and the Fab light chain is formed by the complementary VL and CL domains (VL-CL). Accordingly, the Fab heavy chain and the Fab light chain are complementary to each other; a F(ab)₂, a bivalent fragment comprising two Fabs linked by a disulfide bridge at the hinge region; a Fd fragment consisting of the VH and CH1 domains; a Fv fragment or Fv region consisting of a dimer of one VL and one VH domain. Accordingly, the VH and VL domain of a Fv fragment or Fv region are complementary to each other; a dAb fragment (Ward et al., (1989) Nature 341:544-546), which consists of a VH domain; and an isolated complementarity determining region (CDR). Furthermore, although the two variable domains of the Fv fragment or Fv region, VL and VH, are coded for by separate genes, they can be joined, using recombinant methods, by a synthetic linker that enables them to be made as a single protein chain in which the VL and VH regions pair to form monovalent molecules (referred herein as “single chain Fv” or “scFv”; see e.g., Bird et al., (1988) Science 242:423-426; and Huston et al., (1988) Proc. Natl. Acad. Sci. 85:5879-5883). Such single chain antibodies are also intended to be encompassed within the term “antibody fragment”. These antibody fragments are obtained using conventional techniques known to those of skill in the art, and the fragments are screened for utility in the same manner as are intact antibodies. Antibody fragments can also be incorporated into single domain antibodies, maxibodies, minibodies, intrabodies, diabodies, triabodies, tetrabodies, v-NAR and bis-scFv (see, e.g., Hollinger and Hudson, (2005) Nature Biotechnology 23:1126-1136). Antibody fragments can be grafted into scaffolds based on polypeptides such as Fibronectin type III (Fn3) (see U.S. Pat. No. 6,703,199, which describes fibronectin polypeptide monobodies). Antibody fragments can be incorporated into single chain molecules comprising a pair of tandem Fv segments (VH-CH1-VH-CH1) which, together with complementary light chain polypeptides, form a pair of antigen-binding sites (Zapata et al., (1995) Protein Eng. 8:1057-1062; and U.S. Pat. No. 5,641,870).

A “Fv fragment” or “Fv region” is a monovalent antibody fragment, which consists of a dimer of one VL and one VH domain. Accordingly, the VH and VL domain of an Fv fragment or Fv region are complementary to each other.

A “Fab” or “Fab fragment” is a monovalent antibody fragment consisting of the VL, VH, CL and CH1 domains. The Fab heavy chain consists of one VH and one CH1 domain (VH-CH1) and the Fab light chain consists of one VL and one CL domain (VL-CL). Accordingly, the Fab heavy chain and the Fab light chain are complementary to each other.

The term immunoglobulin (Ig) “hinge” as used herein refers to one of the two polypeptides forming the dimeric “hinge region” of an immunoglobulin. The hinge includes the portion of an immunoglobulin heavy chain that joins the CH1 domain to the CH2 domain. Accordingly, a natural occurring immunoglobulin is composed of two identical hinges, which are linked via one or more disulfide bridges formed through interchain cysteins present in the two hinges. In other words, a natural occurring immunoglobulin is composed of a dimeric disulfide stabilized hinge region, that joins the two Fab arms of an immunoglobulin to the Fc region. A hinge can be subdivided into three distinct domains: upper, middle, and lower hinge (Roux et ah, J. Immunol. 1998 161:4083).

The term “Fc region” as used herein refers to the two Fc region subunits being capable of stable association with each other thus forming the dimeric C-terminal region of an immunoglobulin. Accordingly, the two Fc region subunits ((e.g. the first the second Fc region subunit) are complementary to each other. The Fc region of a regular IgG molecule (and of the antigen-binding molecules according to the present disclosure) exists as a dimer, each subunit of which comprises the CH2 and CH3 IgG heavy chain constant domains. The two subunits of the Fc region are capable of stable association with each other.

A “Fc region subunit” as used herein refers to one of the two polypeptides forming the dimeric Fc region of an immunoglobulin or an antigen-binding molecule according to the present disclosure, i.e. a polypeptide comprising C-terminal constant regions of an immunoglobulin heavy chain, capable of stable self-association. Accordingly, the two Fc region subunits ((e.g. the first the second Fc region subunit) which form the dimeric Fc region are complementary to each other. For example, IgG Fc region subunit comprises an IgG CH2 and an IgG CH3 constant domain. The term includes native sequence Fc regions subunits and variant Fc region subunits. Although the boundaries of the Fc region subunits of an IgG heavy chain might vary slightly, the human IgG heavy chain Fc region subunit is usually defined to extend from Cys226, or from Pro230, to the C-terminus of the heavy chain. However, the C-terminal lysine (Lys447) of the Fc region subunit may or may not be present. Unless otherwise specified herein, numbering of amino acid residues in the Fc region is according to the EU numbering system, also called the EU index, as described in Kabat et al., Sequences of Proteins of Immunological Interest, 5th Ed. Public Health Service, National Institutes of Health, Bethesda, Md., 1991.

A “human antibody” or “human antibody fragment” as used herein, includes antibodies and antibody fragments having variable regions in which both the framework and CDR regions are derived from sequences of human origin. Furthermore, if the antibody contains a constant region, the constant region also is derived from such sequences. Human origin includes, e.g., human germline sequences, or mutated versions of human germline sequences or antibody containing consensus framework sequences derived from human framework sequences analysis, for example, as described in Knappik et al., (2000) J Mol Biol 296:57-86). The structures and locations of immunoglobulin variable domains, e.g., CDRs, may be defined using well known numbering schemes, e.g., the Kabat numbering scheme, the Chothia numbering scheme, or a combination of Kabat and Chothia (see, e.g., Sequences of Proteins of Immunological Interest, U.S. Department of Health and Human Services (1991), eds. Kabat et al.; Lazikani et al., (1997) J. Mol. Bio. 273:927-948); Kabat et al., (1991) Sequences of Proteins of Immunological Interest, 5th edit., NIH Publication no. 91-3242 U.S. Department of Health and Human Services; Chothia et al., (1987) J. Mol. Biol. 196:901-917; Chothia et al., (1989) Nature 342:877-883; and Al-Lazikani et al., (1997) J. Mol. Biol. 273:927-948. Human antibodies and human variable regions can also be isolated from synthetic libraries or from transgenic mice (e.g. xenomouse) provided the respective system yield in antibodies having variable regions in which both the framework and CDR regions are derived from sequences of human origin.

The term “chimeric antibody” or “chimeric antibody fragment” is defined herein as an antibody which has constant antibody regions derived from, or corresponding to, sequences found in one species and variable antibody regions derived from another species. Preferably, the constant antibody regions are derived from, or corresponding to, sequences found in humans, and the variable antibody regions (e.g. VH, VL, CDR or FR regions) are derived from sequences found in a non-human animal, e.g. a mouse, rat, rabbit or hamster.

A “humanized antibody” or “humanized antibody fragment” is defined herein as an antibody molecule which has constant antibody regions derived from sequences of human origin and the variable antibody regions or parts thereof or only the CDRs are derived from another species. Humanization may be achieved by various methods including, but not limited to (a) grafting the non-human (e.g., donor antibody) CDRs onto human (e.g. recipient antibody) framework and constant regions with or without retention of critical framework residues (e.g. those that are important for retaining good antigen binding affinity or antibody functions), (b) grafting only the non-human specificity-determining regions (SDRs or a-CDRs; the residues critical for the antibody-antigen interaction) onto human framework and constant regions, or (c) transplanting the entire non-human variable domains, but “cloaking” them with a human-like section by replacement of surface residues. Humanized antibodies and methods of making them are reviewed, e.g., in Almagro and Fransson, Front Biosci 13, 1619-1633 (2008), and are further described, e.g., in Riechmann et al., Nature 332, 323-329 (1988); Queen et al., Proc Natl Acad Sci USA 86, 10029-10033 (1989); U.S. Pat. Nos. 5,821,337, 7,527,791, 6,982,321, and 7,087,409; Jones et al., Nature 321, 522-525 (1986); Morrison et al., Proc Natl Acad Sci 81, 6851-6855 (1984); Morrison and Oi, Adv Immunol 44, 65-92 (1988); Verhoeyen et al, Science 239, 1534-1536 (1988); Padlan, Molec Immun 31(3), 169-217 (1994); Kashmiri et al., Methods 36, 25-34 (2005) (describing SDR (a-CDR) grafting); Padlan, Mol Immunol 28, 489-498 (1991) (describing “resurfacing”); Dall'Acqua et al, Methods 36, 43-60 (2005) (describing “FR shuffling”); and Osbourn et al, Methods 36, 61-68 (2005) and Klimka et al, Br J Cancer 83, 252-260 (2000) (describing the “guided selection” approach to FR shuffling).

The term “isolated” refers to a compound, which can be e.g. an antibody, antibody fragment or antigen-binding molecule, that is substantially free of other antibodies, antibody fragments or antigen-binding molecules having different antigenic specificities. Moreover, an isolated antibody, antibody fragment or antigen-binding molecule may be substantially free of other cellular material and/or chemicals. Thus, in some embodiments, the antibodies, antibody fragments or antigen-binding molecules provided are isolated antibodies, antibody fragments or antigen-binding molecules that have been separated from antibodies or antigen-binding molecules with a different specificity. An isolated antibody or antigen-binding molecule may be a monoclonal antibody, antibody fragment or antigen-binding molecule. An isolated antibody, antibody fragments or antigen-binding molecule may be a recombinant monoclonal antibody, antibody fragment or antigen-binding molecule. An isolated antibody, antibody fragment or antigen-binding molecule that specifically binds to an epitope, isoform or variant of a target may, however, have cross-reactivity to other related antigens, e.g., from other species (e.g., species homologs).

The term “recombinant antibody”, “recombinant antibody fragment” or “recombinant antigen-binding molecule”, as used herein, includes all antibodies, antibody fragments or antigen-binding molecules according to the present disclosure that are prepared, expressed, created or segregated by means not existing in nature. For example, antibodies or antigen-binding molecules isolated from a host cell transformed to express the antibody or antigen-binding molecule, antibodies selected and isolated from a recombinant, combinatorial human antibody library, and antibodies prepared, expressed, created or isolated by any other means that involve splicing of all or a portion of a human immunoglobulin gene, sequences to other DNA sequences or antibodies isolated from an animal (e.g., a mouse) that is transgenic or transchromosomal for human immunoglobulin genes or a hybridoma prepared therefrom. Preferably, such recombinant antibodies or antigen-binding molecules have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. A recombinant antibody or antigen-binding molecule may be a recombinant monoclonal antibody or a recombinant monoclonal antigen-binding molecule. In an embodiment, the antibodies and antibody fragment disclosed herein are isolated from the Ylanthia® antibody library as disclosed in U.S. Ser. No. 13/321,564 or U.S. Ser. No. 13/299,367, which both herein are incorporated by reference.

As used herein, the term “monoclonal antibody”, “monoclonal antibody fragment” or “monoclonal antigen-binding molecule” refers to an antibody, antibody fragment or antigen-binding molecule disclosed herein that is derived from a single clone, including any eukaryotic, prokaryotic, or phage clone, and not the method by which it is produced. Monoclonal antibodies or antibody fragments may be made by the hybridoma method as described in Kohler et al.; Nature, 256:495 (1975) or may be isolated from phage libraries. Other methods for the preparation of clonal cell lines and monoclonal antibodies or antigen-binding molecule as disclosed herein expressed thereby are well known in the art (see, for example, Chapter 11 in: Short Protocols in Molecular Biology, (2002) 5th Ed., Ausubel et al., eds., John Wiley and Sons, New York).

The term “multispecific” means that an antigen-binding molecule is able to specifically bind to two or more different antigens. Typically, a multispecific antigen-binding molecule comprises of two or more antigen-binding sites, each of which is specific for a different antigen or epitope. The term “bispecific” means that an antibody or antigen-binding molecule is able to specifically bind to two different antigens. Typically, a bispecific antigen-binding molecule comprises two antigen-binding sites, each of which is specific for a different antigen or epitope.

As used herein the term “binds specifically to”, “specifically binds to”, is “specific to/for” or “specifically recognizes”, or the like, refers to measurable and reproducible interactions such as binding between a target antigen and an antibody, antibody fragment or antigen-binding molecule disclosed herein, which is determinative of the presence of the target antigen in the presence of a heterogeneous population of molecules including biological molecules. For example, an antibody, antibody fragment or antigen-binding molecule disclosed herein that specifically binds to a target antigen (which can be an antigen or an epitope of an antigen) is an antibody, antibody fragment, or antigen-binding molecule that binds this target with greater affinity, avidity, more readily, and/or with greater duration than it binds to other target antigens. In certain embodiments, an antibody, antibody fragment or antigen-binding molecule specifically binds to an epitope on a protein that is conserved among the protein from different species. In another embodiment, specific binding can include, but does not require exclusive binding. The antibodies, antibody fragments or antigen-binding molecules disclosed herein specifically bind to antigens. Methods for determining whether two molecules specifically bind are well known in the art and include, for example, a standard ELISA assay. The scoring may be carried out by standard color development (e.g. secondary antibody with horseradish peroxide and tetramethyl benzidine with hydrogen peroxide). The reaction in certain wells is scored by the optical density, for example, at 450 nm. Typical background (=negative reaction) may be 0.1 OD; typical positive reaction may be 1 OD. This means the difference positive/negative can be more than 5-fold. Typically, determination of binding specificity is performed by using not a single reference antigen, but a set of three to five unrelated antigens, such as milk powder, BSA, transferrin or the like.

As used herein, the term “affinity” refers to the strength of interaction between a polypeptide and its target antigen at a single site. Within each site, the binding site of the polypeptide interacts through weak non-covalent forces with its target at numerous sites; the more interactions, the stronger the affinity.

The term “K_{D}”, as used herein, refers to the dissociation constant, which is obtained from the ratio of Kd to Ka (i.e. Kd/Ka) and is expressed as a molar concentration (M). K_{D} values for antigen-binding molecules like e.g. monoclonal antibodies or monoclonal antigen-binding molecules as disclosed herein can be determined using methods well established in the art. Methods for determining the K_{D} of an antigen-binding molecule like e.g. a monoclonal antibody or monoclonal antigen-binding molecule as disclosed herein are SET (soluble equilibrium titration) or surface plasmon resonance using a biosensor system such as a Biacore® system. In the present disclosure an antibody or antigen-binding molecule according to the present disclosure specific for an antigen typically has a dissociation rate constant (KD) (koff/kon) of less than 5×10⁻²M, less than 1×10⁻²M, less than 5×10⁻³M, less than 1×10⁻³M, less than 5×10⁻⁴M, less than 1×10⁻⁴M, less than 5×10⁻⁵M, less than 1×10⁻⁵M, less than 5×10⁻⁶M, less than 1×10⁻⁶M, less than 5×10⁻⁷M, less than 1×10⁻⁷M, less than 5×10⁻⁸M, less than 1×10⁻⁸M, less than 5×10⁻⁹M, less than 1×10⁻⁹M, less than 5×10⁻¹⁰M, less than 1×10⁻¹⁰M, less than 5×10⁻¹¹M, less than 1×10⁻¹¹M, less than 5×10⁻¹²M, less than 1×10⁻¹²M, less than 5×10⁻¹³M, less than 1×10⁻¹³M, less than 5×10⁻¹⁴M, less than 1×10⁻¹⁴M, less than 5×10⁻¹⁵M, or less than 1×10⁻¹⁵M or lower for the antigen.

The term “epitope” refers to a site (e.g. a contiguous stretch of amino acids or a conformational configuration made up of different regions of non-contiguous amino acid residues) on a polypeptide or protein, which is specifically recognized by an antibody, antibody fragment or antigen-binding molecule as disclosed herein, or a T-cell receptor or otherwise interacts with a molecule. Generally, epitopes are of chemically active surface groupings of molecules such as amino acids or carbohydrate or sugar side chains and generally may have specific three-dimensional structural characteristics, as well as specific charge characteristics. As will be appreciated by one of skill in the art, practically anything to which an antibody or antigen-binding molecule can specifically bind could be an epitope. An epitope can comprise those residues to which the antibody or antigen-binding molecule binds and may be “linear” or “conformational.” The term “linear epitope” refers to an epitope wherein all of the points of interaction between the protein and the interacting molecule (such as an antibody) occur linearly along the primary amino acid sequence of the protein (continuous). The term “conformational epitope” refers to an epitope in which discontinuous amino acid residues that come together in three dimensional conformations. In a conformational epitope, the points of interaction occur across amino acid residues on the protein that are separated from one another. For example, an epitope can be one or more amino acid residues within a stretch of amino acid residues as shown by peptide mapping or HDX, or one or more individual amino acid residues as shown by X-ray crystallography.

“Binds the same epitope as” means the ability of an antibody, antibody fragment or antigen-binding molecule to bind to a specific antigen and binding to the same epitope as the exemplified antibody or antigen-binding molecule when using the same epitope mapping technique for comparing the antibodies or antigen-binding molecules. The epitopes of the exemplified antibody, antigen-binding molecules, other antibodies and antigen-binding molecules can be determined using epitope mapping techniques. Epitope mapping techniques are well known in the art. For example, conformational epitopes are readily identified by determining spatial conformation of amino acids such as by, e.g., hydrogen/deuterium exchange, x-ray crystallography and two-dimensional nuclear magnetic resonance.

The terms “engineered” or “modified” as used herein includes manipulation of nucleic acids or polypeptides by synthetic means (e.g. by recombinant techniques, in vitro peptide synthesis, by enzymatic or chemical coupling of peptides or some combination of these techniques). Preferably, the antibodies, antibody fragments or antigen-binding molecules according to the present disclosure are engineered or modified to improve one or more properties, such as antigen binding, stability, half-life, effector function, immunogenicity, safety and the like.

The term “valent” as used herein denotes the presence of a specified number of antigen-binding sites in an antigen-binding molecule.

As used herein, the terms “first” and “second” with respect to a Fab and/or Fv region, Fc region subunit or the like are used for distinguishing when there is more than one of each type of component. Use of these terms is not intended to confer a specific order or orientation of the bispecific antigen binding molecule unless explicitly so stated.

A “modification promoting the association of the first and the second Fc region subunit” is a manipulation of the polypeptide backbone or the post-translational modifications of an Fc region subunit that reduces or prevents the association of a polypeptide comprising the Fc region subunit with an identical polypeptide to form a homodimer. A modification promoting association as used herein particularly includes separate modifications made to each of the two Fc region subunits desired to associate (i.e. the first and the second Fc region subunit), wherein the modifications are complementary to each other so as to promote association of the two Fc region subunits. For example, a modification promoting association may alter the structure or charge of one or both of the Fc region subunits to make their association sterically or electrostatically favorable, respectively. Accordingly, heterodimerization occurs between a polypeptide comprising the first Fc region subunit and a polypeptide comprising the second Fc region subunit, which might be non-identical in the sense that further components fused to each of the subunits (e.g. Fab, Fv) are not the same.

As used herein, “amino acid residues” or “amino acid” will be indicated either by their full name or according to the standard three-letter or one-letter amino acid code. “Natural occurring amino acids” means the following amino acids:

TABLE 1
Natural occurring amino acids

| Amino acid | Three letter code | One letter code |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The term “amino acid mutation” as used herein is meant to encompass amino acid substitutions, deletions, insertions, and modifications. Any combination of substitution, deletion, insertion, and modification can be made as long as the final construct possesses the desired characteristics, e.g., reduced binding to an Fc receptor, or increased association with another peptide. Amino acid sequence deletions and insertions include amino- and/or carboxy-terminal deletions and insertions of amino acid residues. Particular amino acid mutations are amino acid substitutions. Amino acid substitutions include replacement by non-naturally occurring amino acids or by naturally occurring amino acid derivatives of the twenty standard amino acids. Amino acid mutations can be generated using genetic or chemical methods well known in the art. Genetic methods may include site-directed mutagenesis, PCR, gene synthesis and the like. It is contemplated that methods of altering the side chain group of an amino acid residue by methods other than genetic engineering, such as chemical modification, may also be useful. Various designations may be used herein to indicate the same amino acid mutation. For example, a substitution from glyince at position 327 of the Fc region to alanine can be indicated as 237A, G337, G337A, or Gly329Ala.

The term “vector” refers to a polynucleotide molecule capable of transporting another polynucleotide to which it has been linked. Preferred vectors are those capable of autonomous replication and/or expression of nucleic acids to which they are linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and mammalian vectors). Other vectors can be integrated into the genome of a host cell upon introduction into the host cell, and thereby are replicated along with the host genome. Vectors may be compatible with prokaryotic or eukaryotic cells. Prokaryotic vectors typically include a prokaryotic replicon, which may include a prokaryotic promoter capable of directing the expression (transcription and translation) of the peptide in a bacterial host cell, such as Escherichia coli transformed therewith. A promoter is an expression control element formed by a DNA sequence that permits binding of RNA polymerase and transcription to occur. Promoter sequences compatible with bacterial hosts are typically provided in plasmid vectors containing convenience restriction sites for insertion of a DNA segment. Examples of such vector plasmids include pUC8, pUC9, pBR322, and pBR329, pPL and pKK223, available commercially. “Expression vectors” are those vectors capable of directing the expression of nucleic acids to which they are operatively linked and is intended to include such other forms of expression vectors, such as viral vectors (e.g., replication defective retroviruses, adenoviruses and adeno-associated viruses), which serve equivalent functions. The expression vector includes an expression cassette into which the nucleic acid sequence encoding an antigen-binding molecule according to the present disclosure (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region.

As used herein, the term “host cell” refers to any kind of cellular system which can be engineered to generate an antigen-binding molecule according to the present disclosure and refers to a cell into which a (recombinant expression) vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. Typical host cells are prokaryotic (such as bacterial, including but not limited to E. coli) or eukaryotic (which includes yeast, mammalian cells, and more). Bacterial cells are preferred prokaryotic host cells and typically are a strain of Escherichia coli (E. coli) such as, for example, the E. coli strain DH5 available from Bethesda Research Laboratories, Inc., Bethesda, Md. Preferred eukaryotic host cells include yeast and mammalian cells including murine and rodents, preferably vertebrate cells such as those from a mouse, rat, monkey or human cell line, for example HKB11 cells, PERC.6 cells, or CHO cells.

The term “EC₅₀” as used herein, refers to the concentration of an antibody, antibody fragment or antigen-binding molecule as disclosed herein, which induces a response in an assay half way between the baseline and maximum. It therefore represents the antibody or antigen-binding molecule concentration at which 50% of the maximal effect is observed.

The terms “inhibition” or “inhibit” or “reduction” or “reduce” or “neutralization” or “neutralize” refer to a decrease or cessation of any phenotypic characteristic (such as binding, a biological activity or function) or to the decrease or cessation in the incidence, degree, or likelihood of that characteristic. The “inhibition”, “reduction” or “neutralization” needs not to be complete as long as it is detectable using an appropriate assay. In some embodiments, by “reduce” or “inhibit” is meant the ability to cause a decrease of 20% or greater. In another embodiment, by “reduce” or “inhibit” is meant the ability to cause a decrease of 50% or greater. In yet another embodiment, by “reduce” or “inhibit” is meant the ability to cause an overall decrease of 75%, 85%, 90%, 95%, or greater.

The terms “increase” or “enhance” refer to an increase of any phenotypic characteristic (such as binding, a biological activity or function) or to the increase in the incidence, degree, or likelihood of that characteristic. The “increase” or “enhance” needs not to be maximum effect as long as it is detectable using an appropriate assay. In some embodiments, by “increase” or “enhance” is meant the ability to cause an increase of 20% or greater. In another embodiment, by “increase” or “enhance” is meant the ability to cause an increase of 50% or greater. In yet another embodiment, by “increase” or “enhance” is meant the ability to cause an overall increase of 75%, 85%, 90%, 95%, or greater.

The term “antagonistic” antigen-binding molecule as used herein refers to an antigen-binding molecule that interacts with an antigen and partially or fully inhibits or neutralizes a biological activity or function or any other phenotypic characteristic of an target antigen.

The term “agonistic” antigen-binding molecule as used herein refers to an antigen-binding molecule that interacts with an antigen and increases or enhances a biological activity or function or any other phenotypic characteristic of the target antigen.

An “effective amount” of an agent, e.g. a pharmaceutical composition, refers to the amount that is necessary to result in a physiological change in the cell or tissue to which it is administered.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

The terms “individual” or “subject” refer to a mammal.

The term “pharmaceutical composition” refers to a preparation which is in such form as to permit the biological activity of an active ingredient contained therein to be effective, and which contains no additional components which are unacceptably toxic to a subject to which the formulation would be administered.

The term “pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative.

As used herein, “treatment”, “treat” or “treating” and the like refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antigen-binding molecules according to the preset disclosure are used to delay development of a disease or to slow the progression of a disease.

The term “effector function” refers to those biological activities attributable to the Fc region of an antibody or antigen-binding molecules according to the present disclosure, which vary with the antibody isotype. Examples of antibody effector functions include C1q binding and complement dependent cytotoxicity (CDC); Fc receptor binding and antibody-dependent cell-mediated cytotoxicity (ADCC); phagocytosis; down regulation of cell surface receptors (e.g. B cell receptor); and B cell activation.

“Antibody-dependent cell-mediated cytotoxicity” or “ADCC” refers to a form of cytotoxicity in which antibodies or antigen-binding molecules according to the present disclosure bound onto Fc receptors (FcRs) present on certain cytotoxic cells (e.g. NK cells, neutrophils, and macrophages) enable these cytotoxic effector cells to bind specifically to an antigen-bearing target cell and subsequently kill the target cell with cytotoxins. The primary cells for mediating ADCC, NK cells, express FcγRIII only, whereas monocytes express FcγRI, FcγRII, and FcγRIII.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) or to antigen-binding molecules of the present disclosure, which are bound to their cognate antigen. To assess complement activation, a CDC assay, e.g., as described in Gazzano-Santoro et al, J. Immunol. Methods 202:163 (1996), may be performed. Polypeptide variants with altered Fc region subunit amino acid sequences (polypeptides with a variant or modified Fc region subunit) and increased or decreased C1q binding capability are described, e.g., in U.S. Pat. No. 6,194,551 and WO 1999/51642. See also, e.g., Idusogie et al. J. Immunol. 164: 4178-4184 (2000).

### Embodiments

The present disclosure pertains to antigen-binding molecules that are suited to co-engage two or more (different) antigens simultaneously. The ability to target two or more different antigens with different valency (e.g. one antigen monovalently and one antigen bivalently) is a particular useful aspect of the antigen-binding molecules disclosed herein.

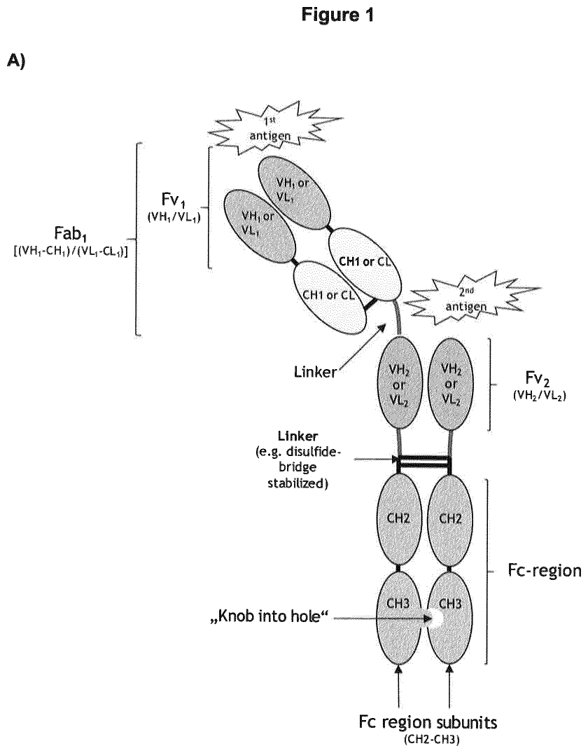

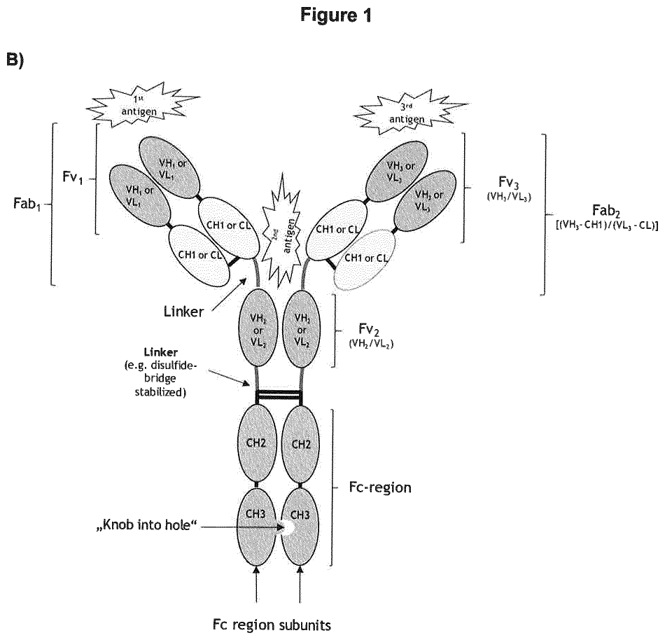

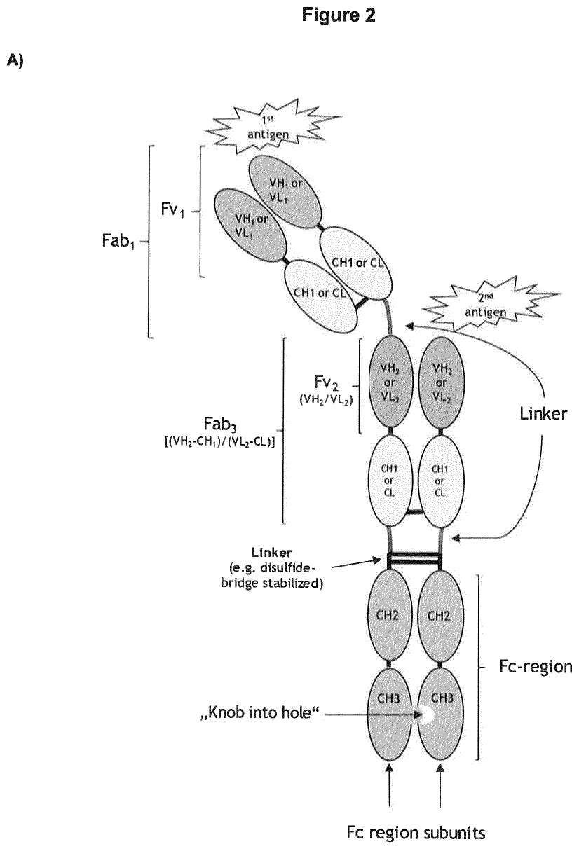

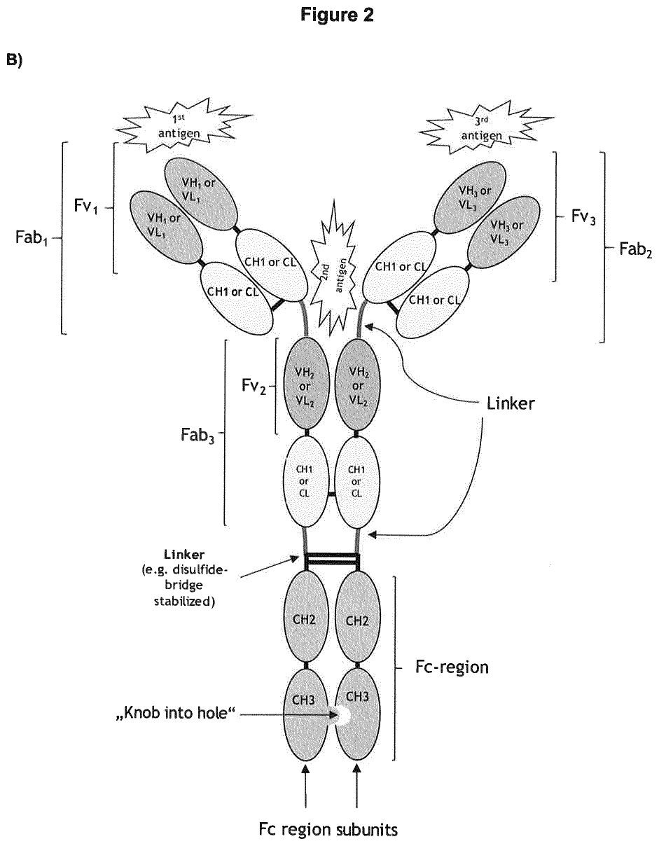

The individual components of an antigen-binding molecule according to the present disclosure can be used to each other in a variety of configurations. Exemplary configurations are depicted in FIGS. 1 and 2.

In general, there are two main types of antigen-binding molecules as described herein:
a. one type that utilizes two Fv regions. This type allows for (I) simultaneous monovalent binding to two (different) antigens or (II) bivalent binding to one antigen.
b. one type that utilizes three Fv regions. This type allows for (I) simultaneous bivalent binding to one antigen and monovalent binding to second antigen or (II) trivalent binding to one antigen.

### Multispecific Antigen-Binding Molecules

An antigen-binding molecule according to the present disclosure can be made of two or more, preferably three Fv regions. Accordingly, the present molecule can act as a bivalent or trivalent antigen-binding molecule. The basic structures of antigen-binding molecules according to the present disclosure are depicted in FIGS. 1 and 2.

### Bivalent Antigen-Binding Molecules

In an embodiment, an antigen-binding molecule according to the present disclosure is composed of two Fv regions. This is achieved by using a regular immunoglobulin (e.g. IgG) antibody structure (which lacks one of the two Fab arms) that incorporates an additional Fv region between the retained Fab arm and the Fc portion of the immunoglobulin structure.

In an embodiment, an antigen-binding molecule according to the present disclosure allows for a monovalent binding to two different antigens. In such an embodiment, the antigen-binding molecule comprises at least two Fv regions, wherein one Fv region binds to a first antigen and the other Fv region binds to a second antigen. In such an embodiment, the antigen-binding molecule according to the present disclosure refers to a bivalent bispecific antigen-binding molecule.

In an embodiment, the present disclosure pertain to an antigen-binding molecule, comprising
a) a first Fab comprising a first Fv region, which specifically binds to a first antigen,
b) a second Fv region which specifically binds to a second antigen and
c) a Fc region composed of a first and second Fc region subunit; wherein
   I. the C-terminus of the heavy or light chain of the first Fab is fused to the N-terminus of the VH or VL of the second Fv region, and wherein
   II. the C-terminus of the VH or VL of the second Fv region is fused to the N-terminus of the first Fc region subunit and the N-terminus of the second Fc domain subunit is fused to the C-terminus of the complementary variable domain of the second Fv region.

In an embodiment, the C-terminus of the heavy chain of the first Fab is fused to the N-terminus of the VH of the second Fv region. In an embodiment, the C-terminus of the CH1 of the first Fab is fused to the N-terminus of the VH of the second Fv region. In an embodiment, the fusion occurs via a peptide linker.

In an embodiment, the C-terminus of the heavy chain of the first Fab is fused to the N-terminus of the VL of the second Fv region. In an embodiment, the C-terminus of the CH1 of the first Fab is fused to the N-terminus of the VL of the second Fv region. In an embodiment, the fusion occurs via a peptide linker.

In an embodiment, the C-terminus of the light chain of the first Fab is fused to the N-terminus of the VH of the second Fv region. In an embodiment, the C-terminus of the CL of the first Fab is fused to the N-terminus of the VH of the second Fv region.

In an embodiment, the fusion occurs via a peptide linker. In an embodiment, the C-terminus of the light chain of the first Fab is fused to the N-terminus of the VL of the second Fv region.

In an embodiment, the C-terminus of the CL of the first Fab is fused to the N-terminus of the VL of the second Fv region. In an embodiment, the fusion occurs via a peptide linker.

In an embodiment, the C-terminus of the VH of the second Fv region is fused to the N-terminus of the first Fc region subunit. In an embodiment, the fusion occurs via a peptide linker. In an embodiment, the C-terminus of the VH of the second Fv region is fused to the N-terminus of the second Fc region subunit. In an embodiment, the fusion occurs via a peptide linker. In an embodiment, the C-terminus of the VL of the second Fv region is fused to the N-terminus of the first Fc region subunit. In an embodiment, the fusion occurs via a peptide linker. In an embodiment, the C-terminus of the VL of the second Fv region is fused to the N-terminus of the second Fc region subunit. In an embodiment, the fusion occurs via a peptide linker. In an embodiment, the N-terminus of the first Fc domain subunit is fused to the C-terminus of the VH of the second Fv region. In an embodiment, the fusion occurs via a peptide linker. In an embodiment, the N-terminus of the first Fc domain subunit is fused to the C-terminus of the VL of the second Fv region. In an embodiment, the fusion occurs via a peptide linker.

In an embodiment, the N-terminus of the second Fc domain subunit is fused to the C-terminus of the VH of the second Fv region. In an embodiment, the fusion occurs via a peptide linker. In an embodiment, the N-terminus of the second Fc domain subunit is fused to the C-terminus of the VL of the second Fv region. In an embodiment, the fusion occurs via a peptide linker.

In an embodiment, the C-terminus of the heavy chain of the first Fab is fused to the N-terminus of the VH of the second Fv region and the C-terminus of the VH of the second Fv region is fused to the N-terminus of the first Fc region subunit. In an embodiment, the C-terminus of the CH1 of the first Fab is fused to the N-terminus of the VH of the second Fv region and the C-terminus of the VH of the second Fv region is fused to the N-terminus of the first Fc region subunit. In an embodiment, each fusion occurs via a peptide linker.

In an embodiment, the C-terminus of the heavy chain of the first Fab is fused to the N-terminus of the VH of the second Fv region and the C-terminus of the VH of the second Fv region is fused to the N-terminus of the second Fc region subunit. In an embodiment, the C-terminus of the CH1 of the first Fab is fused to the N-terminus of the VH of the second Fv region and the C-terminus of the VH of the second Fv region is fused to the N-terminus of the second Fc region subunit. In an embodiment, each fusion occurs via a peptide linker.

In an embodiment, the C-terminus of the heavy chain of the first Fab is fused to the N-terminus of the VH of the second Fv region and the C-terminus of the VL of the second Fv region is fused to the N-terminus of the first Fc region subunit. In an embodiment, the C-terminus of the CH1 of the first Fab is fused to the N-terminus of the VH of the second Fv region and the C-terminus of the VL of the second Fv region is fused to the N-terminus of the first Fc region subunit. In an embodiment, each fusion occurs via a peptide linker.

In an embodiment, the C-terminus of the heavy chain of the first Fab is fused to the N-terminus of the VH of the second Fv region and the C-terminus of the VL of the second Fv region is fused to the N-terminus of the second Fc region subunit. In an embodiment, the C-terminus of the CH1 of the first Fab is fused to the N-terminus of the VH of the second Fv region and the C-terminus of the VL of the second Fv region is fused to the N-terminus of the second Fc region subunit. In an embodiment, each fusion occurs via a peptide linker.

In an embodiment, the C-terminus of the heavy chain of the first Fab is fused to the N-terminus of the VL of the second Fv region and the C-terminus of the VH of the second Fv region is fused to the N-terminus of the first Fc region subunit. In an embodiment, the C-terminus of the CH1 of the first Fab is fused to the N-terminus of the VL of the second Fv region and the C-terminus of the VH of the second Fv region is fused to the N-terminus of the first Fc region subunit. In an embodiment, each fusion occurs via a peptide linker.

In an embodiment, the C-terminus of the heavy chain of the first Fab is fused to the N-terminus of the VL of the second Fv region and the C-terminus of the VH of the second Fv region is fused to the N-terminus of the second Fc region subunit. In an embodiment, the C-terminus of the CH1 of the first Fab is fused to the N-terminus of the VL of the second Fv region and the C-terminus of the VH of the second Fv region is fused to the N-terminus of the second Fc region subunit. In an embodiment, each fusion occurs via a peptide linker.

In an embodiment, the C-terminus of the heavy chain of the first Fab is fused to the N-terminus of the VL of the second Fv region and the C-terminus of the VL of the second Fv region is fused to the N-terminus of the first Fc region subunit. In an embodiment, the C-terminus of the CH1 of the first Fab is fused to the N-terminus of the VL of the second Fv region and the C-terminus of the VL of the second Fv region is fused to the N-terminus of the first Fc region subunit. In an embodiment, each fusion occurs via a peptide linker.

In an embodiment, the C-terminus of the heavy chain of the first Fab is fused to the N-terminus of the VL of the second Fv region and the C-terminus of the VL of the second Fv region is fused to the N-terminus of the second Fc region subunit. In an embodiment, the C-terminus of the CH1 of the first Fab is fused to the N-terminus of the VL of the second Fv region and the C-terminus of the VL of the second Fv region is fused to the N-terminus of the second Fc region subunit. In an embodiment, each fusion occurs via a peptide linker.

In an embodiment, the C-terminus of the light chain of the first Fab is fused to the N-terminus of the VH of the second Fv region and the C-terminus of the VH of the second Fv region is fused to the N-terminus of the first Fc region subunit. In an embodiment, the C-terminus of the CL of the first Fab is fused to the N-terminus of the VH of the second Fv region and the C-terminus of the VH of the second Fv region is fused to the N-terminus of the first Fc region subunit. In an embodiment, each fusion occurs via a peptide linker.

In an embodiment, the C-terminus of the light chain of the first Fab is fused to the N-terminus of the VH of the second Fv region and the C-terminus of the VH of the second Fv region is fused to the N-terminus of the second Fc region subunit. In an embodiment, the C-terminus of the CL of the first Fab is fused to the N-terminus of the VH of the second Fv region and the C-terminus of the VH of the second Fv region is fused to the N-terminus of the second Fc region subunit. In an embodiment, each fusion occurs via a peptide linker.

In an embodiment, the C-terminus of the light chain of the first Fab is fused to the N-terminus of the VH of the second Fv region and the C-terminus of the VL of the second Fv region is fused to the N-terminus of the first Fc region subunit. In an embodiment, the C-terminus of the CL of the first Fab is fused to the N-terminus of the VH of the second Fv region and the C-terminus of the VL of the second Fv region is fused to the N-terminus of the first Fc region subunit. In an embodiment, each fusion occurs via a peptide linker.

In an embodiment, the C-terminus of the light chain of the first Fab is fused to the N-terminus of the VH of the second Fv region and the C-terminus of the VL of the second Fv region is fused to the N-terminus of the second Fc region subunit. In an embodiment, the C-terminus of the CL of the first Fab is fused to the N-terminus of the VH of the second Fv region and the C-terminus of the VL of the second Fv region is fused to the N-terminus of the second Fc region subunit. In an embodiment, each fusion occurs via a peptide linker.

In an embodiment, the C-terminus of the light chain of the first Fab is fused to the N-terminus of the VL of the second Fv region and the C-terminus of the VH of the second Fv region is fused to the N-terminus of the first Fc region subunit. In an embodiment, the C-terminus of the CL of the first Fab is fused to the N-terminus of the VL of the second Fv region and the C-terminus of the VH of the second Fv region is fused to the N-terminus of the first Fc region subunit. In an embodiment, each fusion occurs via a peptide linker.

In an embodiment, the C-terminus of the light chain of the first Fab is fused to the N-terminus of the VL of the second Fv region and the C-terminus of the VH of the second Fv region is fused to the N-terminus of the second Fc region subunit. In an embodiment, the C-terminus of the CL of the first Fab is fused to the N-terminus of the VL of the second Fv region and the C-terminus of the VH of the second Fv region is fused to the N-terminus of the second Fc region subunit. In an embodiment, each fusion occurs via a peptide linker.

In an embodiment, the C-terminus of the light chain of the first Fab is fused to the N-terminus of the VL of the second Fv region and the C-terminus of the VL of the second Fv region is fused to the N-terminus of the first Fc region subunit. In an embodiment, the C-terminus of the CL of the first Fab is fused to the N-terminus of the VL of the second Fv region and the C-terminus of the VL of the second Fv region is fused to the N-terminus of the first Fc region subunit. In an embodiment, each fusion occurs via a peptide linker.

In an embodiment, the C-terminus of the light chain of the first Fab is fused to the N-terminus of the VL of the second Fv region and the C-terminus of the VL of the second Fv region is fused to the N-terminus of the second Fc region subunit. In an embodiment, the C-terminus of the CL of the first Fab is fused to the N-terminus of the VL of the second Fv region and the C-terminus of the VL of the second Fv region is fused to the N-terminus of the second Fc region subunit. In an embodiment, each fusion occurs via a peptide linker.

In an embodiment, the C-terminus of the heavy chain of the first Fab is fused to the N-terminus of the VH of the second Fv region and the C-terminus of the VH of the second Fv region is fused to the N-terminus of the first Fc region subunit and the N-terminus of the second Fc region subunit is fused to the C-terminus of the VL of the second Fv region. In an embodiment, the C-terminus of the CH1 of the first Fab is fused to the N-terminus of the VH of the second Fv region and the C-terminus of the VH of the second Fv region is fused to the N-terminus of the first Fc region subunit and the N-terminus of the second Fc region subunit is fused to the C-terminus of the VL of the second Fv region. In an embodiment, each fusion occurs via a peptide linker.

In an embodiment, the C-terminus of the heavy chain of the first Fab is fused to the N-terminus of the VH of the second Fv region and the C-terminus of the VH of the second Fv region is fused to the N-terminus of the second Fc region subunit and the N-terminus of the first Fc region subunit is fused to the C-terminus of the VL of the second Fv region. In an embodiment, the C-terminus of the CH1 of the first Fab is fused to the N-terminus of the VH of the second Fv region and the C-terminus of the VH of the second Fv region is fused to the N-terminus of the second Fc region subunit and the N-terminus of the first Fc region subunit is fused to the C-terminus of the VL of the second Fv region. In an embodiment, each fusion occurs via a peptide linker.

In an embodiment, the C-terminus of the heavy chain of the first Fab is fused to the N-terminus of the VH of the second Fv region and the C-terminus of the VL of the second Fv region is fused to the N-terminus of the first Fc region subunit and the N-terminus of the second Fc domain subunit is fused to the C-terminus of the VH of the second Fv region. In an embodiment, the C-terminus of the CH1 of the first Fab is fused to the N-terminus of the VH of the second Fv region and the C-terminus of the VL of the second Fv region is fused to the N-terminus of the first Fc region subunit and the N-terminus of the second Fc domain subunit is fused to the C-terminus of the VH of the second Fv region. In an embodiment, each fusion occurs via a peptide linker.

In an embodiment, the C-terminus of the heavy chain of the first Fab is fused to the N-terminus of the VH of the second Fv region and the C-terminus of the VL of the second Fv region is fused to the N-terminus of the second Fc region subunit and the N-terminus of the first Fc domain subunit is fused to the C-terminus of the VH of the second Fv region. In an embodiment, the C-terminus of the CH1 of the first Fab is fused to the N-terminus of the VH of the second Fv region and the C-terminus of the VL of the second Fv region is fused to the N-terminus of the second Fc region subunit and the N-terminus of the first Fc domain subunit is fused to the C-terminus of the VH of the second Fv region. In an embodiment, each fusion occurs via a peptide linker.

In an embodiment, the C-terminus of the heavy chain of the first Fab is fused to the N-terminus of the VL of the second Fv region and the C-terminus of the VH of the second Fv region is fused to the N-terminus of the first Fc region subunit and the N-terminus of the second Fc region subunit is fused to the C-terminus of the VL of the second Fv region. In an embodiment, the C-terminus of the CH1 of the first Fab is fused to the N-terminus of the VL of the second Fv region and the C-terminus of the VH of the second Fv region is fused to the N-terminus of the first Fc region subunit and the N-terminus of the second Fc region subunit is fused to the C-terminus of the VL of the second Fv region. In an embodiment, each fusion occurs via a peptide linker.

In an embodiment, the C-terminus of the heavy chain of the first Fab is fused to the N-terminus of the VL of the second Fv region and the C-terminus of the VH of the second Fv region is fused to the N-terminus of the second Fc region subunit and the N-terminus of the first Fc region subunit is fused to the C-terminus of the VL of the second Fv region. In an embodiment, the C-terminus of the CH1 of the first Fab is fused to the N-terminus of the VL of the second Fv region and the C-terminus of the VH of the second Fv region is fused to the N-terminus of the second Fc region subunit and the N-terminus of the first Fc region subunit is fused to the C-terminus of the VL of the second Fv region. In an embodiment, each fusion occurs via a peptide linker.

In an embodiment, the C-terminus of the heavy chain of the first Fab is fused to the N-terminus of the VL of the second Fv region and the C-terminus of the VL of the second Fv region is fused to the N-terminus of the first Fc region subunit and the N-terminus of the second Fc region subunit is fused to the C-terminus of the VH of the second Fv region. In an embodiment, the C-terminus of the CH1 of the first Fab is fused to the N-terminus of the VL of the second Fv region and the C-terminus of the VL of the second Fv region is fused to the N-terminus of the first Fc region subunit and the N-terminus of the second Fc region subunit is fused to the C-terminus of the VH of the second Fv region. In an embodiment, each fusion occurs via a peptide linker.

In an embodiment, the C-terminus of the heavy chain of the first Fab is fused to the N-terminus of the VL of the second Fv region and the C-terminus of the VL of the second Fv region is fused to the N-terminus of the second Fc region subunit and the N-terminus of the first Fc region subunit is fused to the C-terminus of the VH of the second Fv region. In an embodiment, the C-terminus of the CH1 of the first Fab is fused to the N-terminus of the VL of the second Fv region and the C-terminus of the VL of the second Fv region is fused to the N-terminus of the second Fc region subunit and the N-terminus of the first Fc region subunit is fused to the C-terminus of the VH of the second Fv region. In an embodiment, each fusion occurs via a peptide linker.

In an embodiment, the C-terminus of the light chain of the first Fab is fused to the N-terminus of the VH of the second Fv region and the C-terminus of the VH of the second Fv region is fused to the N-terminus of the first Fc region subunit and the N-terminus of the second Fc region subunit is fused to the C-terminus of the VL of the second Fv region. In an embodiment, the C-terminus of the CL of the first Fab is fused to the N-terminus of the VH of the second Fv region and the C-terminus of the VH of the second Fv region is fused to the N-terminus of the first Fc region subunit and the N-terminus of the second Fc region subunit is fused to the C-terminus of the VL of the second Fv region. In an embodiment, each fusion occurs via a peptide linker.

In an embodiment, the C-terminus of the light chain of the first Fab is fused to the N-terminus of the VH of the second Fv region and the C-terminus of the VH of the second Fv region is fused to the N-terminus of the second Fc region subunit and the N-terminus of the first Fc region subunit is fused to the C-terminus of the VL of the second Fv region. In an embodiment, the C-terminus of the CL of the first Fab is fused to the N-terminus of the VH of the second Fv region and the C-terminus of the VH of the second Fv region is fused to the N-terminus of the second Fc region subunit and the N-terminus of the first Fc region subunit is fused to the C-terminus of the VL of the second Fv region. In an embodiment, each fusion occurs via a peptide linker.

In an embodiment, the C-terminus of the light chain of the first Fab is fused to the N-terminus of the VH of the second Fv region and the C-terminus of the VL of the second Fv region is fused to the N-terminus of the first Fc region subunit and the N-terminus of the second Fc region subunit is fused to the C-terminus of the VH of the second Fv region. In an embodiment, the C-terminus of the CL of the first Fab is fused to the N-terminus of the VH of the second Fv region and the C-terminus of the VL of the second Fv region is fused to the N-terminus of the first Fc region subunit and the N-terminus of the second Fc region subunit is fused to the C-terminus of the VH of the second Fv region. In an embodiment, each fusion occurs via a peptide linker.

In an embodiment, the C-terminus of the light chain of the first Fab is fused to the N-terminus of the VH of the second Fv region and the C-terminus of the VL of the second Fv region is fused to the N-terminus of the second Fc region subunit and the N-terminus of the first Fc region subunit is fused to the C-terminus of the VH of the second Fv region. In an embodiment, the C-terminus of the CL of the first Fab is fused to the N-terminus of the VH of the second Fv region and the C-terminus of the VL of the second Fv region is fused to the N-terminus of the second Fc region subunit and the N-terminus of the first Fc region subunit is fused to the C-terminus of the VH of the second Fv region. In an embodiment, each fusion occurs via a peptide linker.

In an embodiment, the C-terminus of the light chain of the first Fab is fused to the N-terminus of the VL of the second Fv region and the C-terminus of the VH of the second Fv region is fused to the N-terminus of the first Fc region subunit and the N-terminus of the second Fc region subunit is fused to the C-terminus of the VL of the second Fv region. In an embodiment, the C-terminus of the CL of the first Fab is fused to the N-terminus of the VL of the second Fv region and the C-terminus of the VH of the second Fv region is fused to the N-terminus of the first Fc region subunit and the N-terminus of the second Fc region subunit is fused to the C-terminus of the VL of the second Fv region. In an embodiment, each fusion occurs via a peptide linker.

In an embodiment, the C-terminus of the light chain of the first Fab is fused to the N-terminus of the VL of the second Fv region and the C-terminus of the VH of the second Fv region is fused to the N-terminus of the second Fc region subunit and the N-terminus of the first Fc region subunit is fused to the C-terminus of the VL of the second Fv region. In an embodiment, the C-terminus of the CL of the first Fab is fused to the N-terminus of the VL of the second Fv region and the C-terminus of the VH of the second Fv region is fused to the N-terminus of the second Fc region subunit and the N-terminus of the first Fc region subunit is fused to the C-terminus of the VL of the second Fv region. In an embodiment, each fusion occurs via a peptide linker.

In an embodiment, the C-terminus of the light chain of the first Fab is fused to the N-terminus of the VL of the second Fv region and the C-terminus of the VL of the second Fv region is fused to the N-terminus of the first Fc region subunit and the N-terminus of the second Fc region subunit is fused to the C-terminus of the VH of the second Fv region. In an embodiment, the C-terminus of the CL of the first Fab is fused to the N-terminus of the VL of the second Fv region and the C-terminus of the VL of the second Fv region is fused to the N-terminus of the first Fc region subunit and the N-terminus of the second Fc region subunit is fused to the C-terminus of the VH of the second Fv region. In an embodiment, each fusion occurs via a peptide linker.

In an embodiment, the C-terminus of the light chain of the first Fab is fused to the N-terminus of the VL of the second Fv region and the C-terminus of the VL of the second Fv region is fused to the N-terminus of the second Fc region subunit and the N-terminus of the first Fc region subunit is fused to the C-terminus of the VH of the second Fv region. In an embodiment, the C-terminus of the CL of the first Fab is fused to the N-terminus of the VL of the second Fv region and the C-terminus of the VL of the second Fv region is fused to the N-terminus of the second Fc region subunit and the N-terminus of the first Fc region subunit is fused to the C-terminus of the VH of the second Fv region. In an embodiment, each fusion occurs via a peptide linker.

In an embodiment, an antigen-binding molecule according to the present disclosure is composed of at least 3 polypeptides, wherein
a. a first polypeptide comprises the light or heavy chain of the first Fab,
b. a second polypeptide comprises from its N-terminus to its C-terminus
   i. the complementary light or heavy chain of the first Fab,
   ii. the VH or VL of the second Fv region and
   iii. the first or second Fc region subunit
c. a third polypeptide comprises from its N-terminus to its C-terminus
   i. the complementary VH or VL of the second Fv region and
   ii. the complementary first or second Fc region subunit.

In an embodiment, the first polypeptide comprises the light chain of the first Fab. In an embodiment, the first polypeptide comprises the heavy chain of the first Fab.

In an embodiment, the second polypeptide comprises from its N-terminus to its C-terminus
i. the complementary heavy chain of the first Fab,
ii. the VH of the second Fv region and
iii. the first Fc region subunit.

In an embodiment, the second polypeptide comprises from its N-terminus to its C-terminus
i. the complementary heavy chain of the first Fab,
ii. the VL of the second Fv region and
iii. the first Fc region subunit.

In an embodiment, the second polypeptide comprises from its N-terminus to its C-terminus
i. the complementary heavy chain of the first Fab,
ii. the VH of the second Fv region and
iii. the second Fc region subunit.

In an embodiment, the second polypeptide comprises from its N-terminus to its C-terminus
i. the complementary heavy chain of the first Fab,
ii. the VL of the second Fv region and
iii. the second Fc region subunit.

In an embodiment, the second polypeptide comprises from its N-terminus to its C-terminus
i. the complementary light chain of the first Fab,
ii. the VH of the second Fv region and
iii. the first Fc region subunit.

In an embodiment, the second polypeptide comprises from its N-terminus to its C-terminus
i. the complementary light chain of the first Fab,
ii. the VL of the second Fv region and
iii. the first Fc region subunit.

In an embodiment, the second polypeptide comprises from its N-terminus to its C-terminus
i. the complementary light chain of the first Fab,
ii. the VH of the second Fv region and
iii. the second Fc region subunit.

In an embodiment, the second polypeptide comprises from its N-terminus to its C-terminus
i. the complementary light chain of the first Fab,
ii. the VL of the second Fv region and
iii. the second Fc region subunit.

In an embodiment, the third polypeptide comprises from its N-terminus to its C-terminus
i. the complementary VH of the second Fv region and
ii. the complementary first Fc region subunit.

In an embodiment, the third polypeptide comprises from its N-terminus to its C-terminus
i. the complementary VH of the second Fv region and
ii. the complementary second Fc region subunit.

In an embodiment, the third polypeptide comprises from its N-terminus to its C-terminus
i. the complementary VL of the second Fv region and
ii. the complementary first Fc region subunit.

In an embodiment, the third polypeptide comprises from its N-terminus to its C-terminus
i. the complementary VL of the second Fv region and
ii. the complementary second Fc region subunit.

In an embodiment, an antigen-binding molecule according to the present disclosure is composed of at least 3 polypeptides, wherein
a. a first polypeptide comprises the light chain of the first Fab,
b. a second polypeptide comprises from its N-terminus to its C-terminus
   i. the complementary heavy chain of the first Fab,
   ii. the VL of the second Fv region and
   iii. the first Fc region subunit
c. a third polypeptide comprises from its N-terminus to its C-terminus
i. the complementary VH of the second Fv region and
ii. the complementary second Fc region subunit.

In an embodiment, an antigen-binding molecule according to the present disclosure is composed of at least 3 polypeptides, wherein
a. a first polypeptide comprises the light chain of the first Fab,
b. a second polypeptide comprises from its N-terminus to its C-terminus
   i. the complementary heavy chain of the first Fab,
   ii. the VH of the second Fv region and
   iii. the first Fc region subunit
c. a third polypeptide comprises from its N-terminus to its C-terminus
   i. the complementary VL of the second Fv region and
   ii. the complementary second Fc region subunit.

In an embodiment, an antigen-binding molecule according to the present disclosure is composed of 3 polypeptides.

According to the aforementioned embodiments, the first Fv region and the second Fv region can be of different antigen specificity and are fused to each other in a configuration, which allows for a geometry and distance between the two Fv regions different from that of the bispecific antibody formats known in the art.

An antigen-binding molecule according to the present disclosure provides monovalent binding to at least one of the antigens it binds to. Monovalent binding may be desired or required in situations where internalization of the target antigen occurs following bivalent binding of an antigen-binding molecule. In such cases, bivalent binding to one target antigen may enhance internalization of the antigen, thereby reducing its availability. Furthermore, monovalent binding is essential where crosslinking of a target antigen is not desired. For example, bivalent binding to certain target classes, such as receptor tyrosine kinase, may mimic the function of natural ligands resulting in receptor activation rather an inactivation.

The configuration present in an antigen-binding molecule according to the present disclosure is particularly suited to mimic the immunological synapse between a T-cell and a target cell, as required, if a bispecific antigen-binding molecule is to be used for T-cell engagement and redirection. Ensuring monovalent binding to an activating T-cell antigen (such as CD3) minimizes the risk of activation of said T-cell in the absence of target cells.

However, bivalent binding to a target antigen might be also desirable in certain situations, for example to increase binding affinity and to optimize targeting.

### Trivalent Antigen-Binding Molecules

In an preferred embodiment, an antigen-binding molecule according to the present disclosure is composed of three Fv regions. This is achieved by using a regular immunoglobulin (e.g. IgG) antibody structure (two heavy chains with associated two light chains that form two Fv regions) that incorporates an additional Fv region between the two Fab arms and the Fc portion of the regular immunoglobulin structure.

In an embodiment, an antigen-binding molecule according to the present disclosure comprises a second Fab comprising a third Fv region, which specifically binds to a third antigen.

In an embodiment, the third antigen is identical to the first or second antigen. In an embodiment, the third antigen is identical to the first antigen. In an embodiment, the third antigen is identical to the second antigen.

In an embodiment, an antigen-binding molecule according to the present disclosure comprises a second Fab comprising a third Fv region, which specifically binds to the first or the second antigen.

In an embodiment, an antigen-binding molecule according to the present disclosure comprises a first Fab comprising a first Fv region, which specifically binds to a first antigen, a second Fab comprising a third Fv region, which specifically binds to a third antigen and a second Fv region, which specifically binds to a second antigen. In an embodiment, the third antigen is identical to the first or second antigen. In an embodiment, the third antigen is identical to the first antigen.

In an embodiment, the second Fab is fused to the second Fv region. In an embodiment, the C-terminus of the second Fab is fused to the N-terminus of the second Fv region. In an embodiment, the second Fab is fused to the second Fv region via a peptide linker.

In an embodiment, the present disclosure provides an antigen-binding molecule, comprising
a) a first Fab comprising a first Fv region, which specifically binds to a first antigen,
b) a second Fv region which specifically binds to a second antigen and
c) a second Fab comprising a third Fv region, which specifically binds to a third antigen, and
d) a Fc region composed of a first and second Fc region subunit; wherein
   I. the C-terminus of the heavy or light chain of the first Fab is fused to the N-terminus of the VH or VL of the second Fv region, and wherein
      II. the C-terminus of the VH or VL of the second Fv region is fused to the N-terminus of the first Fc region subunit and the N-terminus of the second Fc domain subunit is fused to the C-terminus of the complementary variable domain of the second Fv region, and wherein
      III. the C-terminus of the heavy or light chain of the second Fab is fused to the N-terminus of the VH or VL of the second Fv region with the proviso that the first and second Fab are fused to distinct variable domains of the second Fv region.

In an embodiment, each fusion occurs via a peptide linker.

In an embodiment, the antigen-binding molecule according to the present disclosure comprises not more than three Fv domains. In an embodiment, the antigen-binding molecule according to the present disclosure consists of three Fv domains.

In an embodiment, the C-terminus of the CH1 or CL of the second Fab is fused to the N-terminus of the VH or VL of the second Fv region with the proviso that the first and second Fab are fused to distinct variable domains of the second Fv region.

In an embodiment, the C-terminus of the heavy chain of the second Fab is fused to the N-terminus of the VH of the second Fv region with the proviso that first and second Fab are fused to distinct variable domains of the second Fv region. In an embodiment, the C-terminus of the CH1 of the second Fab is fused to the N-terminus of the VH of the second Fv region with the proviso that first and second Fab are fused to distinct variable domains of the second Fv region. In an embodiment, the fusion occurs via a peptide linker.

In an embodiment, the C-terminus of the heavy chain of the second Fab is fused to the N-terminus of the VL of the second Fv region with the proviso that first and second Fab are fused to distinct variable domains of the second Fv region. In an embodiment, the C-terminus of the CH1 of the second Fab is fused to the N-terminus of the VL of the second Fv region with the proviso that first and second Fab are fused to distinct variable domains of the second Fv region. In an embodiment, the fusion occurs via a peptide linker.

In an embodiment, the C-terminus of the light chain of the second Fab is fused to the N-terminus of the VH of the second Fv region with the proviso that first and second Fab are fused to distinct variable domains of the second Fv region. In an embodiment, the C-terminus of the CL of the second Fab is fused to the N-terminus of the VH of the second Fv region with the proviso that first and second Fab are fused to distinct variable domains of the second Fv region. In an embodiment, the fusion occurs via a peptide linker.

In an embodiment, the C-terminus of the light chain of the second Fab is fused to the N-terminus of the VL of the second Fv region with the proviso that first and second Fab are fused to distinct variable domains of the second Fv region. In an embodiment, the C-terminus of the CL of the second Fab is fused to the N-terminus of the VL of the second Fv region with the proviso that first and second Fab are fused to distinct variable domains of the second Fv region. In an embodiment, the fusion occurs via a peptide linker.

In an embodiment, the C-terminus of the heavy chain of the first Fab is fused to the N-terminus of the VL of the second Fv region and the C-terminus of the heavy chain of the second Fab is fused to the N-terminus of the VH of the second Fv region. In an embodiment, the C-terminus of the heavy chain of the first Fab is fused to the N-terminus of the VH of the second Fv region and the C-terminus of the heavy chain of the second Fab is fused to the N-terminus of the VL of the second Fv region.

In an embodiment, the C-terminus of the light chain of the first Fab is fused to the N-terminus of the VL of the second Fv region and the C-terminus of the light chain of the second Fab is fused to the N-terminus of the VH of the second Fv region. In an embodiment, the C-terminus of the light chain of the first Fab is fused to the N-terminus of the VL of the second Fv region and the C-terminus of the light chain of the second Fab is fused to the N-terminus of the VL of the second Fv region. In an embodiment, the fusion occurs via a peptide linker.

In an embodiment, the C-terminus of the heavy chain of the first Fab is fused to the N-terminus of the VL of the second Fv region and the C-terminus of the light chain of the second Fab is fused to the N-terminus of the VH of the second Fv region. In an embodiment, the C-terminus of the heavy chain of the first Fab is fused to the N-terminus of the VH of the second Fv region and the C-terminus of the light chain of the second Fab is fused to the N-terminus of the VL of the second Fv region. In an embodiment, the C-terminus of the light chain of the first Fab is fused to the N-terminus of the VL of the second Fv region and the C-terminus of the heavy chain of the second Fab is fused to the N-terminus of the VH of the second Fv region. In an embodiment, the C-terminus of the light chain of the first Fab is fused to the N-terminus of the VH of the second Fv region and the C-terminus of the heavy chain of the second Fab is fused to the N-terminus of the VL of the second Fv region. In an embodiment, the fusion occurs via a peptide linker.

In an embodiment, the antigen-binding molecule according to the present disclosure is composed of at least 4 polypeptides, wherein
a. a first polypeptide comprises the light or heavy chain of the first Fab,
b. a second polypeptide comprises from its N-terminus to its C-terminus
   i. the complementary light or heavy chain of the first Fab,
   ii. the VH or VL of the second Fv region and
   iii. the first or second Fc domain subunit
c. a third polypeptide comprises from its N-terminus to its C-terminus
   i. the light or heavy chain of the second Fab,
   ii. the complementary VH or VL of the second Fv region and
   iii. the complementary first or second Fc domain subunit
d. a fourth polypeptide comprises the complementary light or heavy chain of the second Fab.

In an embodiment, the first polypeptide comprises the light chain of the first Fab. In an embodiment, the light chain of the first Fab comprises the VL and CL of the first Fab. In an embodiment, the first polypeptide comprises the heavy chain of the first Fab. In an embodiment, the heavy chain of the first Fab comprises the VH and CH1 of the first Fab.

In an embodiment, the second polypeptide comprises from its N-terminus to its C-terminus
i. the complementary heavy chain of the first Fab,
ii. the VH of the second Fv region and
iii. the first Fc region subunit.

In an embodiment, the second polypeptide comprises from its N-terminus to its C-terminus
i. the complementary heavy chain of the first Fab,
ii. the VL of the second Fv region and
iii. the first Fc region subunit.

In an embodiment, the second polypeptide comprises from its N-terminus to its C-terminus
i. the complementary heavy chain of the first Fab,
ii. the VH of the second Fv region and
iii. the second Fc region subunit.

In an embodiment, the second polypeptide comprises from its N-terminus to its C-terminus
i. the complementary heavy chain of the first Fab,
ii. the VL of the second Fv region and
iii. the second Fc region subunit.

In an embodiment, the second polypeptide comprises from its N-terminus to its C-terminus
i. the complementary light chain of the first Fab,
ii. the VH of the second Fv region and
iii. the first Fc region subunit.

In an embodiment, the second polypeptide comprises from its N-terminus to its C-terminus
i. the complementary light chain of the first Fab,
ii. the VL of the second Fv region and
iii. the first Fc region subunit.

In an embodiment, the second polypeptide comprises from its N-terminus to its C-terminus
i. the complementary light chain of the first Fab,
ii. the VH of the second Fv region and
iii. the second Fc region subunit.

In an embodiment, the second polypeptide comprises from its N-terminus to its C-terminus
i. the complementary light chain of the first Fab,
ii. the VL of the second Fv region and
iii. the second Fc region subunit.

In an embodiment, the third polypeptide comprises from its N-terminus to its C-terminus
i. the heavy chain of the second Fab,
ii. the complementary VH of the second Fv region and
iii. the complementary first Fc region subunit.

In an embodiment, the third polypeptide comprises from its N-terminus to its C-terminus
i. the heavy chain of the second Fab,
ii. the complementary VH of the second Fv region and
iii. the complementary second Fc region subunit.

In an embodiment, the third polypeptide comprises from its N-terminus to its C-terminus
i. the heavy chain of the second Fab,
ii. the complementary VL of the second Fv region and
iii. the complementary first Fc region subunit.

In an embodiment, the third polypeptide comprises from its N-terminus to its C-terminus
i. the heavy chain of the second Fab,
ii. the complementary VL of the second Fv region and
iii. the complementary second Fc region subunit.

In an embodiment, the third polypeptide comprises from its N-terminus to its C-terminus
i. the light chain of the second Fab,
ii. the complementary VH of the second Fv region and
iii. the complementary first Fc region subunit.

In an embodiment, the third polypeptide comprises from its N-terminus to its C-terminus
i. the light chain of the second Fab,
ii. the complementary VH of the second Fv region and
iii. the complementary second Fc region subunit.

In an embodiment, the third polypeptide comprises from its N-terminus to its C-terminus
i. the light chain of the second Fab,
ii. the complementary VL of the second Fv region and
iii. the complementary first Fc region subunit.

In an embodiment, the third polypeptide comprises from its N-terminus to its C-terminus
i. the light chain of the second Fab,
ii. the complementary VL of the second Fv region and
iii. the complementary second Fc region subunit.

In an embodiment, the fourth polypeptide comprises the complementary light chain of the second Fab. In an embodiment, the fourth polypeptide comprises the complementary heavy chain of the second Fab. In an embodiment, an antigen-binding molecule according to the present disclosure is composed of 4 polypeptides. In an embodiment, an antigen-binding molecule according to the present disclosure, is composed of at least 4 polypeptides, wherein
a. a first polypeptide comprises the light chain of the first Fab,
b. a second polypeptide comprises from its N-terminus to its C-terminus
   i. the complementary heavy chain of the first Fab,
   ii. the VL of the second Fv region and
   iii. the first Fc domain subunit
c. a third polypeptide comprises from its N-terminus to its C-terminus
   i. the heavy chain of the second Fab,
   ii. the complementary VH of the second Fv region and
   iii. the complementary second Fc domain subunit
d. a fourth polypeptide comprises the complementary light chain of the second Fab.

In an embodiment, an antigen-binding molecule according to the present disclosure, is composed of at least 4 polypeptides, wherein
a. a first polypeptide comprises the light chain of the first Fab,
b. a second polypeptide comprises from its N-terminus to its C-terminus
   i. the complementary heavy chain of the first Fab,
   ii. the VH of the second Fv region and
   iii. the first Fc domain subunit
c. a third polypeptide comprises from its N-terminus to its C-terminus
   i. the heavy chain of the second Fab,
   ii. the complementary VL of the second Fv region and
   iii. the complementary second Fc domain subunit
d. a fourth polypeptide comprises the complementary light chain of the second Fab.

In an embodiment, the light chain of the first or second Fab comprises the VL and CL of the first or second Fab, respectively. In an embodiment, the light chain of the first or second Fab consists of the VL and CL of the first or second Fab, respectively.

In an embodiment, the third antigen is identical to the first antigen.

In an embodiment, the antigen-binding molecule according to the present disclosure provides bivalent binding to the first antigen and monovalent binding to the second antigen.

In an embodiment, the antigen-binding molecule according to the present disclosure is a trivalent bispecific antigen-binding molecule.

In an embodiment, the first antigen is a tumor-associated antigen. In an embodiment, the first antigen is a tumor-associated antigen expressed on a tumor cell.

In an embodiment, the second antigen is an immune cell related antigen. In an embodiment, the second antigen is expressed on an immune cell. In an embodiment, the second antigen is expressed on an immune effector cell. In an embodiment, the second antigen is expressed on a cytotoxic T-cell. In an embodiment, the second antigen is CD3.

In an embodiment, the Fc region is an IgG1 Fc region. In an embodiment, said IgG1 Fc region is a human IgG1 Fc region. In an embodiment, the Fc region comprises one or more amino acid modifications promoting the association of the first and second Fc region subunit.

In an embodiment, in the CH3 domain of first Fc region subunit, the threonine residue at position 366 is replaced with a tryptophan residue (T366W) and the serine residue at position 354 is replaced with a cysteine residue (S354C) and in the CH3 domain of the second Fc region subunit the tyrosine residue at position 407 is replaced with a valine residue (Y407V), the threonine residue at position 366 is replaced with a serine residue (T366S), the leucine residue at position 368 is replaced with an alanine residue (L368A) and the tyrosine residue at position 349 is replaced by a cysteine residue (Y349C) with numbering according EU index.

In an embodiment, in each Fc region subunit, at least 5 amino acid residues in the positions corresponding to positions L234, L235, G237, A330, P331 with numbering according EU index in a human IgG1 are mutated to A, E, A, S, and S, respectively.

### Antibodies

The antibodies or antibody fragments, or heavy and light chain variable regions used in an antigen-binding molecule according to the present disclosure can be of any animal species origin, such as murine, rat, human or non-human primate. Preferably, the origin is human or may be also obtained by humanization approaches.

Accordingly, the Fab and/or Fv regions used in the antigen-binding molecules according to the present disclosure are human or humanized. In an embodiment, the Fv region is human. In an embodiment, the Fv region is humanized. In an embodiment, the Fab is human. In an embodiment, the Fab is humanized. In yet another embodiment, the Fab comprises human heavy and light chain constant regions.

### Linkers

An antigen-binding molecule according to the present disclosure can be designed such that its individual components (e.g. Fab, Fv region, Fc region, variable domains, constant domains) are fused directly to each other or indirectly through a linker.

In certain embodiments, the individual components of an antigen-binding molecule according to the present disclosure are genetically fused to each other. Such fusion can be achieved by a number of strategies, which include, but are not limited to polypeptide fusion between the N- and C-terminus of polypeptides, fusion via disulfide bonds, and fusion via chemical cross-linking reagents.

A variety of linkers may be used in the embodiments described herein to covalently fuse the individual components of an antigen-binding molecule according to the present disclosure to its intended fusion partner. The composition and length of a linker may be determined in accordance with methods well known in the art and may be tested for efficacy. Preferably, the linker is non-immunogenic. In an embodiment, the linker is a peptide linker. In an embodiment, the linker is a peptide linker comprising one or more amino acid residues, joined by peptide bonds that are known in the art. The peptide linker should have a length that is adequate to fuse two polypeptides (or components) in such a way that they assume the correct conformation relative to one another so that they retain or obtain the desired activity.

In an embodiment, a peptide linker according to the present disclosure is from 1 to 70 amino residues in length, 1 to 50 amino acid residues in length, 1 to 40 amino residues in length, 1 to 30 amino acid residues in length, 1 to 20 amino acid residues in length, 1 to 10 amino acid residue in length, 1 to 5 amino acid residues in length.

In an embodiment, a peptide linker according to the present disclosure has a length of at least 1, 2, 3, 4, 5, 6, 7, 8, 9, 10, 11, 12, 13, 14, 15, 16, 17, 18, 19, 20, 25, 30, 35, 40, 45, 50, 60, 70 amino acids residues. In an embodiment, a peptide linkers linker according to the present disclosure has a length of 1, 2, 3, 4, 5, 6, 7, 8, 9, 10, 11, 12, 13, 14, 15, 16, 17, 18, 19, 20, 25, 30, 35, 40, 45, 50, 60, or 70 amino acids residues

The peptide linker may pre-dominantly comprise the following amino acid residues: Gly, Ser, Ala, or Thr. Suitable, non-immunogenic peptide linkers comprises glycine-serine polymers for example, (GS)ₙ (SEQ ID NO: 34), (G₄S)ₙ (SEQ ID NO: 35), (SG₄)ₙ (SEQ ID NO: 36), (GSGGS)ₙ (SEQ ID NO: 37), (GGGS)ₙ (SEQ ID NO: 38) or G₄(SG₄)ₙ (SEQ ID NO: 39), wherein n is an integer between 1 and 10, typically between 2 and 4. A non-immunogenic peptide linker used herein may comprise glycine-alanine polymers, alanine-serine polymers, and other flexible peptide linkers. A suitable peptide linker for fusing the first Fab and/or the second Fab to the second Fv region is a glycine-serine polymers, such as (GGS)₃ (SEQ ID NO: 10).

Peptide linkers can be also derived from immunoglobulin light or heavy chain constant domain, such as CLκ or CLλ domains or the CH1 domain, but not all residues of such a constant domain, for example only the first 5-12 amino acid residues. In an embodiment, the peptide linkers is not a immunoglobulin light or heavy chain constant domain. In an embodiment, the peptide linker is not a CLκ, CLλ, CH1, CH2 or CH3 domain.

Exemplary peptide linkers which may be used in an antigen-binding molecule are derived from immunoglobulin light or heavy chain constant domain are QPKAAP (SEQ ID NO: 12) or ASTKGP (SEQ ID NO: 11). In general, peptide linkers can be derived from immunoglobulin heavy chains of any isotype, including for example Cy1, Cy2, Cy3, Cy4, Ca1, Ca2, C8, Cs, and Oμ.

A peptide linker may also comprise an immunoglobulin hinge (e.g. a human IgG1 hinge or part thereof) or any peptide derived from such hinge. Preferably, where only a part or portion of an immunoglobulin hinge is used, the truncated hinge may still include one or more of its interchain cysteines. The presence of the interchain cysteines allows for the formation of a dimeric peptide linker (or hinge region) by disulfide bridges, in situations where two of such hinge peptide linkers are used. A preferred situation for the use of such disulfide stabilized dimeric peptide linkers is the fusion of the variable domains of the second Fv region to the Fc domain subunits. The presence of a dimeric-peptide linker or hinge region additionally promotes and stabilizes the dimerization of the two Fc region subunits present in an antigen-binding molecule according to the present disclosure. An exemplary human IgG hinge derived peptide linker suited for dimerization is DKTHTCPPCP (SEQ ID NO: 13).

It is understood that a peptide linker as used herein is not limited to only one of the aforementioned and exemplified peptide linkers but my comprise any combination of two or more such linker which are fused to each other. For instance, a peptide linker as used herein may be built from a glycine-serine polymer and an immunoglobulin hinge derived sequence in order to retain or obtain the desired activity.

Alternatively, a variety of non-proteinaceous polymers, including but not limited to polyethylene glycol (PEG), polypropylene glycol, polyoxyalkylenes, or copolymers of polyethylene glycol and polypropylene glycol, may be used as linkers.

### The Fc Region

The Fc region of an antigen-binding molecule according to the present disclosure consists of a pair of polypeptides comprising heavy chain domains of a regular immunoglobulin. The Fc region of a regular IgG exists as a dimer, each subunit of which comprises the CH2 and CH3 IgG heavy chain constant domains. The two Fc region subunits are capable of stable association with each other. Accordingly, in an embodiment, the two Fc region subunits of an antigen-binding molecule according to the present disclosure are capable of stable association with each other. In an embodiment, the Fc region of an antigen-binding molecule according to the present disclosure is an IgG Fc region. In an embodiment, the Fc region is an IgG1 Fc region. In an embodiment, the Fc region is human. In an embodiment, the Fc region is a human IgG1 Fc region.

### The Heterodimeric Fc Region

The two Fc region subunits of an antigen-binding molecule according to the present disclosure are typically comprised in two non-identical polypeptide chains. To improve the yield and purity of the molecule in recombinant production, it is advantageous to introduce in the Fc region one or more modifications promoting the association of the two non-identical polypeptides forming the Fc region subunits. Accordingly, in certain embodiments, the present disclosure provides heterodimeric antigen-binding molecules that rely on the use of two different variant Fc region subunits that will self-assemble to form a heterodimeric molecule.

In an embodiment, the Fc region of an antigen-binding molecule according to the present disclosure comprises one or more modifications promoting the association of the first and the second Fc region subunit. In an embodiment, the first and second Fc region subunit of an antigen-binding molecule according to the present disclosure comprises one or more modification promoting the association of the first and the second Fc region subunit. In an embodiment, the first Fc region subunit and second Fc region subunit comprises one or more modification that reduce homodimerization or reduce homodimer formation between two identical polypeptide chains comprising the same Fc region subunit.

A modification may be present in the first Fc region subunit and/or the second Fc region subunit. In a preferred embodiment, a modification is present in the first and second Fc region subunit. In one embodiment, said modification occurs in the CH3 domain of an Fc region subunit. A modification can be made by altering the nucleic acid encoding the polypeptides, e.g. by site-specific mutagenesis, or by peptide synthesis. Several approaches for CH3 modifications in order to promote heterodimerization have been described, for example in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, WO 2013/096291, which are herein incorporated by reference.

Typically, in the heterodimerization approaches known in the art, the CH3 domain of one polypeptide chain (e.g. immunoglobulin heavy chain) and the CH3 domain of the other polypeptide chain are both engineered in a complementary manner so that the polypeptide comprising one engineered CH3 domain can no longer homodimerize with another polypeptide chain of the same structure. Thereby the polypeptide comprising one engineered CH3 domain is forced to heterodimerize with the other polypeptide comprising the CH3 domain, which is engineered in a complementary manner.

One heterodimerization approach known in the art is the so-called “knobs-into-holes” technology, which is described in detail providing several examples in e.g. WO 96/027011, Ridgway, J. B., et al, Protein Eng. 9 (1996) 617-621; Merchant, A. M., et al, Nat. Biotechnol. 16 (1998) 677-681; U.S. Pat. Nos. 5,731,168; 7,695,936; WO 98/050431, Carter, J Immunol Meth 248, 7-15 (2001) which are incorporated by reference. The “knobs-into-holes” technology broadly involves: (1) mutating the CH3 domains in each Fc region subunit to promote heterodimerization; and (2) combining the mutated Fc region subunits under conditions that promote heterodimerization. “Knobs” or “protuberances” are typically created by replacing a small amino acid in a parental antibody with a larger amino acid (e.g., T366Y or T366W); “Holes” or “cavities” are created by replacing a larger residue in a parental antibody with a smaller amino acid (e.g., Y407T, T366S, L368A and/or Y407V) with numbering according EU index.

In an embodiment, the modification present in the Fc region of an antigen-binding molecule according to the present disclosure is a “knobs-into-holes” modification, comprising “knob mutations” in one of the two Fc region subunits and “hole mutations” in the other complementary Fc region subunit. The knob modifications and hole modifications can be made by altering the nucleic acid encoding the polypeptides, e.g. by site-specific mutagenesis, or by peptide synthesis. In an embodiment, the CH3 domain of each Fc region subunit is modified according to the knobs-into-holes technology.

In an embodiment, in the CH3 domain of the first Fc region subunit, the threonine residue at position 366 is replaced with a tryptophan residue (T366W) and in the CH3 domain of the second Fc region subunit the tyrosine residue at position 407 is replaced with a valine residue (Y407V) with numbering according EU index. In an embodiment, in the CH3 domain of the second Fc region subunit, the threonine residue at position 366 is replaced with a serine residue (T366S) and the leucine residue at position 368 is replaced with an alanine residue (L368A) with numbering according EU index.

In an embodiment, in the CH3 domain of the first Fc region subunit, the serine residue at position 354 is replaced with a cysteine residue (S354C), and in the CH3 domain of the second Fc region subunit the tyrosine residue at position 349 is replaced by a cysteine residue (Y349C) with numbering according EU index based. Introduction of these two cysteine residues results in formation of a disulfide bridge between the two Fc region subunits, further stabilizing the dimer (Carter, J Immunol Methods 248, 7-15 (2001)).

In a more specific embodiment, the present disclosure provides an antigen-binding molecule, wherein in the CH3 domain of first Fc region subunit, the threonine residue at position 366 is replaced with a tryptophan residue (T366W) and the serine residue at position 354 is replaced with a cysteine residue (S354C) and in the CH3 domain of the second Fc region subunit the tyrosine residue at position 407 is replaced with a valine residue (Y407V), the threonine residue at position 366 is replaced with a serine residue (T366S), the leucine residue at position 368 is replaced with an alanine residue (L368A) and the tyrosine residue at position 349 is replaced by a cysteine residue (Y349C) with numbering according EU index.

In an alternative embodiment, a modification promoting the association of the first and the second Fc region subunit comprises a modification mediating electrostatic steering effects, e.g. as described in PCT publication WO 2009/089004. Generally, this method involves replacement of one or more amino acid residues at the interface of the two Fc region subunits by charged amino acid residues so that homodimer formation becomes electrostatically unfavorable but heterodimerization electrostatically favorable.

### Fc Binding

The Fc region of an immunoglobulin generally confers to the favorable pharmacokinetic properties of antibodies, such as prolonged half-life in serum and to the ability to mediate effector function via binding to Fc receptors expressed on cells. On the other hand, binding to Fc receptors might also results in an undesirable activation of certain cell surface receptors leading to unwanted cytokine release and severe side effects upon systemic administration.

Accordingly, in certain embodiments, the Fc region of an antigen-binding molecule according to the present disclosure is engineered to have an altered binding affinity to an Fc receptor and/or to C1q or to have altered effector function, as compared to a non-engineered Fc region.

Altered effector function can include, but is not limited to, one or more of the following: altered complement dependent cytotoxicity (CDC), altered antibody-dependent cell-mediated cytotoxicity (ADCC), altered antibody-dependent cellular phagocytosis (ADCP), altered cytokine secretion, altered immune complex-mediated antigen uptake by antigen-presenting cells, altered binding to NK cells, altered binding to macrophages, altered binding to monocytes, altered binding to polymorphonuclear cells, altered direct signaling inducing apoptosis, altered crosslinking of target-bound antibodies, altered dendritic cell maturation, or altered T cell priming. In particular embodiments, the altered effector function is one or more selected from the group consisting of CDC, ADCC and ADCP. In an embodiment, the altered effector function is ADCC. In an embodiment, the altered effector function is CDC. In an embodiment, the altered effector function is ADCP. In an embodiment, the altered effector function is CDC, ADCC and ADCP.

Altered effector functions are typically achieved by mutating at least one, preferably both, of the parental Fc domain subunits. Substitutions that result in increased binding as well as decreased binding can be useful. For altering the binding properties of an Fc region, non-conservative amino acid substitutions, i.e. replacing one amino acid with another amino acid having different structural and/or chemical properties, are preferred.

Decreased Fc Receptor Binding and/or Effector Function

For certain therapeutic situations, it may be desirable to reduce or inhibit the normal binding of the Fc region to one or more or all of the Fc receptors and/or binding to a complement component, such as C1q. For instance, it may be desirable to reduce or prevent the binding of an Fc region to one or more or all of the Fcγ receptors (e.g. FcγRI, FcγRIIa, FcγRIIb, FcγRIIIa).

In particular, when an antigen-binding molecule co-engages a receptor of an immune effector cell, it is advisable to prevent FcγRIIIa binding to abolish or significantly reduce ADCC activity and/or to prevent C1q binding to eliminate or significantly reduce CDC activity.

The reduced or abolished effector function can include, but is not limited to, one or more of the following: reduced complement dependent cytotoxicity (CDC), reduced or abolished antibody-dependent cell-mediated cytotoxicity (ADCC), reduced or abolished antibody-dependent cellular phagocytosis (ADCP), reduced or abolished cytokine secretion, reduced or abolished immune complex-mediated antigen uptake by antigen-presenting cells, reduced or abolished binding to NK cells, reduced or abolished binding to macrophages, reduced or abolished binding to monocytes, reduced or abolished binding to polymorphonuclear cells, reduced or abolished direct signaling inducing apoptosis, reduced or abolished crosslinking of target-bound antibodies, reduced or abolished dendritic cell maturation, or reduced or abolished T cell priming. In certain embodiments, the reduced or abolished effector function is one or more selected from the group consisting of CDC, ADCC and ADCP. In an embodiment, the reduced or abolished effector function is ADCC. In an embodiment, the reduced or abolished effector function is CDC. In an embodiment, the reduced or abolished effector function is ADCP. In an embodiment, the reduced or abolished effector function is CDC, ADCC and ADCP.

In an embodiment, the Fc region of an antigen-binding molecule according to the present disclosure is engineered to have a reduced binding affinity to an Fc receptor and/or to C1q and/or to have reduced effector function when compared to a non-engineered Fc region.

In an embodiment, the Fc region of an antigen-binding molecule according to the present disclosure is engineered to have reduced effector function when compared to a non-engineered Fc region. In an embodiment, the Fc region of an antigen-binding molecule according to the present disclosure comprises one or more amino acid mutation that reduces the binding affinity of the Fc region to an Fc receptor and/or to C1q and/or reduces the effector function. In general, the same one or more amino acid mutation(s) is present in each of the two Fc region subunits forming the Fc region. In an embodiment, the one or more amino acid mutations reduces the binding affinity of the Fc region to an Fc receptor. Where there is only one amino acid mutation that reduces the binding affinity of the Fc region to the Fc receptor and/or to C1q, the one amino acid mutation reduces the binding affinity of the Fc region to an Fc receptor and/or to C1q by at least 2-fold, at least 5-fold, or at least 10-fold and/or reduces the effector function by at least 2-fold, at least 5-fold, or at least 10-fold when compared to the non-engineered Fc region. Where there is more than one amino acid mutation that reduces the binding affinity of the Fc region to the Fc receptor and/or to C1q, the combination of these amino acid mutations may reduce the binding affinity of the Fc region to an Fc receptor and/or to C1q by at least 10-fold, at least 20-fold, or at least 50-fold and/or may reduce the effector function by at least 10-fold, at least 20-fold, or at least 50-fold when compared to the non-engineered Fc region.

In an embodiment, the engineered Fc region does substantially not bind to an Fc receptor and/or C1q and/or induce effector function. In an embodiment, the Fc receptor is a human Fc receptor. In one embodiment, the Fc receptor is an activating Fc receptor. In an embodiment, the Fc receptor is an Fcγ receptor. In an embodiment, the Fc receptor is an activating human Fcγ receptor, more specifically human FcγRIIIa, FcγRI or FcγRIIa, most specifically human FcγRIIIa.

In an embodiment, the binding affinity of the Fc region to a complement component, in particular the binding affinity to C1q, is reduced or abolished. In an embodiment, the reduced or abolished effector function is one or more selected from the group of reduced or abolished CDC, reduced or abolished ADCC and reduced or abolished ADCP. In a particular embodiment, the reduced or abolished effector function is reduced ADCC, CDC, and ADCP.

In an embodiment, the Fc region of an antigen-binding molecule according to the present disclosure comprises one or more amino acid mutation(s) that reduce(s) the binding affinity of the Fc region to an Fc receptor and/or to C1q and/or reduces the effector function.

In an embodiment, the amino acid mutation is an amino acid substitution. In an embodiment, the Fc region of an antigen-binding molecule according to the present disclosure comprises one or more amino acid mutations that reduces the binding affinity of the Fc region to an Fc receptor and/or to C1q and/or reduces the effector function, wherein each Fc region subunit comprises an amino acid substitution at a position selected from the group of 234, 235, 237, 330 and 331 with numbering according EU index.

In an embodiment, each Fc region subunit of an antigen-binding molecule according to the present disclosure comprises an amino acid substitution at a position selected from the group of L234, L235 and G237 (numbering according EU index). In an embodiment, each Fc subunit comprises the amino acid substitutions L234A and L235E with numbering according EU index. In an embodiment, each Fc region subunit comprises the amino acid substitutions L234A, L235E and G237A with numbering according EU index. In an embodiment, each Fc region subunit comprises an amino acid substitution at a position selected from the group of 330 and 331 with numbering according EU index. In an embodiment, each Fc region subunit comprises an amino acid substitution at the positions 330 and 331 with numbering according EU index. In an embodiment, the amino acid substitution is A330S or P331S.

In an embodiment, the Fc region of an antigen-binding molecule according to the present disclosure comprises one or more amino acid mutations in each Fc region subunit that reduces the binding affinity of the Fc region to an Fc receptor and/or to C1q and/or reduces the effector function, wherein said one or more amino acid mutations are L234A, L235E, G237A, A330S and P331S.

In an embodiment, the Fc region of an antigen-binding molecule according to the present disclosure consists of one or more amino acid mutation in each Fc region subunit that reduces the binding affinity of the Fc region to an Fc receptor and/or to C1q and/or reduces the effector function, wherein the one or more amino acid mutations are L234A, L235E, G237A, A330S and P331S. In an embodiment, the Fc region is an IgG1 Fc region, particularly a human IgG1 Fc region.

Mutant Fc regions or Fc region subunits can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Increased Fc Receptor Binding and/or Effector Function

In certain situations, it may me desirable to enhance or increase Fc receptor binding and/or C1q binding and/or effector function of an antigen-binding molecule according to the present disclosure.

In an embodiment, the increased or enhanced effector function is one or more selected from the group of CDC, ADCC, ADCP. In an embodiment, the increased or enhanced effector function is ADCC. In an embodiment, the increased or enhanced effector function is CDC. In an embodiment, the increased or enhanced effector function is ADCP. In an embodiment, the increased or enhanced effector function is ADCC, ADCP and CDC. Accordingly, in certain embodiments, the Fc region of an antigen-binding molecule according to the present disclosure is engineered to have an increased binding affinity to an Fc receptor and/or to C1q and/or to have increased effector function when compared to the non-engineered Fc region.

Accordingly, in an embodiment, the Fc region of an antigen-binding molecule according to the present disclosure is engineered to have an increased binding affinity to an Fc receptor when compared the non-engineered Fc region. In an embodiment, the Fc region of an antigen-binding molecule according to the present disclosure is engineered to have an increased binding affinity to C1q when compared the non-engineered Fc region. In certain embodiments, the Fc region of an antigen-binding molecule according to the present disclosure is engineered to have increased effector function when compared to the non-engineered Fc region.

In an embodiment, the Fc region of an antigen-binding molecule according to the present disclosure comprises one or more amino acid mutations in each Fc region subunit that increase the binding affinity of the Fc region to an Fc receptor and/or to C1q and/or increases the effector function. Increased binding affinity may be an increase in the binding affinity of the Fc region to the Fc receptor and/or C1q by at least 2-fold, at least 5-fold, or at least 10-fold when compared to the non-engineered Fc region. Typically, the same amino acid mutations are present in each of the two Fc region subunits. In an embodiment, the one or more amino acid mutation increases the binding affinity of the Fc region to an Fc receptor when compared to the non-engineered Fc region. In an embodiment, the one or more amino acid mutation increases the binding affinity of the Fc region to C1q when compared to the non-engineered Fc region. Examples of amino acid mutations, which result in an increase in binding affinity of an Fc region to an Fc receptor and/or C1q are described in WO 2000/042072 or WO 2004/099249, which are incorporated by reference.

Typically, an amino acid mutation that increases the binding affinity of the Fc region to an Fc receptor and/or to C1q and/or increases effector function is an amino acid substitution.

In embodiments, where there is only one amino acid mutation that increases the binding affinity of the Fc region to the Fc receptor and/or to C1q, the one amino acid mutation may increase the binding affinity of the Fc region to an Fc receptor and/or to C1q by at least 2-fold, at least 5-fold, or at least 10-fold and/or may increase the effector function by at least 2-fold, at least 5-fold, or at least 10-fold when compared to the non-engineered Fc region. In embodiments, where there is more than one amino acid mutation that increases the binding affinity of the Fc region to the Fc receptor and/or to C1q, the combination of these amino acid mutations may increase the binding affinity of the Fc region to an Fc receptor and/or C1q by at least 10-fold, at least 20-fold, or at least 50-fold and/or may increase the effector function by at least 10-fold, at least 20-fold, or at least 50-fold when compared to the non-engineered Fc region.

In an embodiment, the Fc receptor is a human Fc receptor. In an embodiment, the Fc receptor is an activating Fc receptor. In an embodiment, the Fc receptor is a Fcγ receptor. In an embodiment, the Fc receptor is an activating human Fcγ receptor, more specifically human FcγRIIIa, FcγRI or FcγRIIa. In an embodiment, the Fc receptor is selected from the group of FcγRIIIa, FcγRI and FcγRIIa. In a particular embodiment, the Fc receptor is FcγRIIIa.

In an embodiment, the increased effector function is one or more selected from the group of increased ADCC, increased CDC and increased ADCP. In an embodiment, the increased effector function is increased ADCC.

### In Vitro Methods to Assess Binding to Fc Receptors or to Assess Immune Effector Function

Binding of the Fc region to Fc receptors can be easily determined e.g. by ELISA, or by Surface Plasmon Resonance (SPR) using standard instrumentation such as a BIAcore instrument (GE Healthcare), and Fc receptors may be obtained by recombinant expression. Alternatively, the binding affinity of Fc regions may be evaluated using cell lines known to express particular Fc receptors, such as NK cells expressing FcγIIIa receptor. Effector function of an Fc region can be measured by methods known in the art. Suitable in vitro assays to assess ADCC activity of a molecule of interest are for instance described in WO2012130831. Useful effector cells for such assays include peripheral blood mononuclear cells (PBMC) and Natural Killer (NK) cells. Alternatively, or additionally, ADCC activity of the molecule of interest may be assessed in vivo, e.g. in an animal model such as that disclosed in Clynes et al., Proc Natl Acad Sci USA 95, 652-656 (1998). To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)). C1q binding assays (such as ELISA) may be carried out to determine whether an antigen-binding molecule is able to bind C1q and hence has CDC activity (WO 2006/029879 and WO 2005/100402).

### Target Antigens

The novel antigen-binding molecules according to the present disclosure are suited for targeting a variety of antigens and are particularly suited for targeting different antigens simultaneously.

“Antigen” or “target antigen” as used herein refers to any molecule of interest that specifically binds to one of the Fv regions present in an antigen-binding binding molecule according to the present disclosure. Typically, an antigen is a peptide, a protein or any other proteinaceous molecule. Alternatively, an antigen may be any other organic or inorganic molecule, such as carbohydrate, fatty acid, lipid, dye or flourophor.

The ability of an antigen-binding molecule according to the present disclosure to specifically bind to an target antigen can be measured either through an enzyme-linked immunosorbent assay (ELISA) or other techniques familiar to one of skill in the art, e.g. surface plasmon resonance technique (analyzed on a BIACORE T100 system) (Liljeblad, et al., Glyco J 17, 323-329 (2000)), and traditional binding assays (Heeley, Endocr Res 28, 217-229 (2002)).

Competition assays may be used to identify an antibody, antibody fragment, antigen-binding domain or variable domain that cross-competes with a reference antibody for binding to a specific antigen or epitope. “Cross competes” means the ability of an antibody, antibody fragment or antigen-binding molecules to interfere with the binding of other antibodies, antibody fragments or antigen-binding molecules to a specific antigen in a standard competitive binding assay. The ability or extent to which an antibody, antibody fragment or antigen-binding molecule is able to interfere with the binding of another antibody, antibody fragment or antigen-binding molecule to a specific antigen, and, therefore whether it can be said to cross-compete according to the present disclosure, can be determined using standard competition binding assays. One suitable assay involves the use of the Biacore technology (e.g. by using the BIAcore 3000 instrument (Biacore, Uppsala, Sweden)), which can measure the extent of interactions using surface plasmon resonance technology. Another assay for measuring cross-competing uses an ELISA-based approach. A high throughput process for “epitope binning” antibodies based upon their cross-competition is described in International Patent Application No. WO 2003/48731. In certain embodiments, such a competing antibody or antigen-binding-molecule binds to the same epitope (e.g. a linear or a conformational epitope) that is bound by the reference antibody or antigen-binding molecule.

Accordingly, an antigen-binding molecule according to the present disclosure preferably targets two or more, even more preferably, two different antigens (e.g. a first and a second antigen). The two antigens can be expressed on the surface of one cell or can be expressed on the surface of different cells. The ability to target two different antigens with different valency (e.g. one antigen monovalently and one antigen bivalently) is a particular useful aspect of an antigen-binding molecule according to the present disclosure. As outlined before, for some immune receptors (such as the CD3 signaling receptor on T cells) receptor activation only upon binding to the co-target (e.g. a tumor-associated antigen) is desired, because non-specific cross-linking in a clinical setting can result in a life-threatening cytokine storm. By binding such immune receptors monovalently, receptor activation will only occur in response of cross-linking to the co-target.

In an embodiment, the first and/or the second antigen is an antigen associated with a pathological condition, such as an antigen presented on a tumor cell, on a virus-infected cell, or an antigen expressed at a site of inflammation. In an embodiment, the first or second antigen is preferably an antigen expressed on immune cells, such as T-cells. Other suitable antigens include cell surface antigens (such as cell surface receptors), antigens free in blood serum, and/or antigens in the extracellular matrix. In an embodiment, the antigen is a human antigen.

In an embodiment, the first antigen is a tumor-associated antigen, specifically an antigen presented on a tumor cell or a cell of the tumor stroma. In an embodiment, the first antigen is a HLA-restricted peptide. In an embodiment, the first antigen is a peptide/HLA-A0201 complex. The term “HLA-A0201” refer to a specific HLA serotype. HLA-A0201 is a heterodimeric protein, comprising an alpha chain and a beta chain. In an embodiment, the peptide/HLA-A0201 complex is expressed on a cancer cell. In an embodiment, the peptide/HLA-A0201 complex is specific for a cancer cell. In an embodiment, the first antigen is a cancer specific HLA-restricted peptide expressed on the surface of a cancer cell

Non-limiting examples of (tumor-associated) antigens include antigens such as AR, AGR2, A1G1, AKAP1, AKAP2, ANGPT1, ANGPT2, ANPEP, ANGPTL3, APOC1, ANGPTL4, AITGAV, AZGP1, BMP6, BRCA1, BAD, BAG1, BCL2, BL6R, BA2, BPAG1, CDK2, CD52, CD20, CD19, CD3, CD4, CD8, CD164, CDKN1A, CDKN1B, CDKN1C, CDKN2A, CDKN2B, CDKN2C, CDKN3, CDK3, CDK4, CDK5, CDK6, CDK7, CDK9, CLDN3, CLN3, CYB5, CYC1, CCL2, CXCL1, CXCL10, CXCL3, CXCL5, CXCL6, CXCL9, CHGB, CDH20, CDH7, CDH8, CDH9, CD44, CDH1, CDH10, CDH19, CDH20, CDH7, CDH9, CDH13, CDH18, CDH19, CANT1, CAV1, CDH12, CD164, COL6A1, CCL2, CDH5, COL18A1, CHGA, CHGB, CLU, COLIA1, COL6A1, CCNA1, CCNA2, CCND1, CCNE1, CCNE2, COL6A1, CTNNB1, CTSB, CLDN7, CLU, CD44APC, COL4A3, DSfHA, DAB2JP, DES, DNCL1, DD2, DL2, EL24, EGF, E2F1, EGFR, ENO1, ERBB2, ESR1, ESR2, EL2, EStHA, ELAC2, ENO2, ENO3, ERBB2, ESR1, ESR2, EDG1, EFNA1, EFNA3, EFNB2, EPHB4, ESR1, ESR2, EGF, ERK8, EL12A, EL1A, EL24, ENHA, ELK, ECGF1, EREG, EDG1, ENG, E-cadherin, FGF1, FGF10, FGF11, FGF12, FGF13, FGF14, FGF16, FGF17, FGF18, FGF19, FGF2, FGF20, FGF21, FGF22, FGF23, FGF3, FGF4, FGF5, FGF6, FGF7, FGF8, FGF9, FASN, FLJ12584, FLJ25530, F1GF, FLT1, FGFR3, F3, FOSL1, FLRT1, IL12A, IL1A, IL1B, IL2, INHA, IGF1, IGF2, IL12A, IL1A, IL1B, IL2, INHA, IGF1R, IL2, IGFBP6, IL1A, IL1B, IGFBP3, IGFBP6, INSL4, IL6ST, ITGA6, IGF1, IGF2, INSL3, INSL4, IFNA1, IFNB1, IFNG, IL1B, IL6, IGFBP2, IL2RA, IL6, IGF1, IGF2, IGFBP3, IGFBP6, ITGA1, IGF1, ITGA6, ITGB4, INSL3, INSL4, IL29, IL8, ITGB3, GRP, GNRH1, GAGEB1, GAGEC1, GGT1, GSTP1, GATA3, GABRP, GNAS1, GSN, H1P1, HUMCYT2A, HGF, JAG1, JUN, LAMA5, S100A2, SCGB1D2, SCGB2A1, SCGB2A2, SPRR1B, SHBG, SERP1NA3, SHBG, SLC2A2, SLC33A1, SLC43A1, STEAP, STEAP2, SERP1NF1, SERPINB5, SERPINE1, STAB1, TGFA, TGFB1, TGFB2, TGFB3, TNF, TNFSF10, TGFB1I1, TP53, TPM1, TPM2, TRPC6, TGFA, THBS, TEE, TNFRSF6, TNFSF6, TOP2A, TP53, THBS1, THBS2, THBS4, TNFAIP2, TP53, TEK, TGFA, TGFB1, TGFB2, TGFBR1, TGFA, TEV1P3, TGFB3, TNFA1P2, 1TGB3, THBS1, THBS2, VEGF, VEGFC, ODZ1, PAWR, PLG, PAP, PCNA, PRKCQ, PRKD1, PRL, PECAM1, PF4, PROK2, PRL, PAP, PLAU, PRL, PSAP, PART1, PATE, PCA3, P1AS2, PGF, PGR, PLAU, PGR, PLXDCI, PTEN, PTGS2, PDGF, MYC, MMP2, MMP9, MSMB, MACMARCK5, MT3, MUC1, MAP2K7, MKi67, MTSS1, M1B1, MDK, NOX5, NR6A1, NR1H3, NR1I3, NR2F6, NR4A3, NR1H2, NR1H4, NR1I2, NR2C1, NR2C2, NR2E1, NR2E3, NR2F1, NR2F2, NR3C1, NR3C2, NR4A1, NR4A2, NR5A1, NR5A2, NR6A1, NROB1, NROB2, NR1D2, NR1D1, NTN4, NRP1, NRP2, NGFB, NGFR, NME1, KLK6, KLK10, KLK12, KLK13, KLK14, KLK15, KLK3, KLK4, KLK5, KLK6, KLK9, K6HF, KA2, KRT2A, KLK6, KLK3, KRT1, KDR, KLK5, KRT19, KLF5, KRT19, KRTHB6, RARB, RAC2, and ROBO2.

In an embodiment, the first or second antigen is selected from the group of HER2 and CD3. In an embodiment, the first antigen is HER2, particularly human HER2. In an embodiment, the first antigen is CD3, particularly human CD3. In an embodiment, the second antigen is HER2, particularly human HER2. In an embodiment, the second antigen is CD3, particularly human CD3. In an embodiment, the first and/or third Fv region can compete with monoclonal antibody Trastuzumab for binding to an epitope of HER2.

In an embodiment, the first and/or third Fv region specifically binds to HER2 and comprises a VH sequence that is at least 80%, 85%, 90%, 95%, 96%, 97%, 98%, 99% or 100% identical to SEQ ID NO: 8 and a VL sequence that is at least 80%, 85%, 90%, 95%, 96%, 97%, 98%, 99% or 100% identical to SEQ ID NO: 9, or variants thereof that retain functionality.

In an embodiment, the first or second antigen is expressed on an immune cell. In an embodiment, the second antigen is expressed on a T-cell. In an embodiment, the first antigen is expressed on a T-cell.

In an embodiment, the first or second antigen is selected from the group consisting of CD137 and CD3. In an embodiment, the first antigen is selected from the group consisting of CD137 and CD3. In an embodiment, the second antigen is selected from the group consisting of CD137 and CD3. In an embodiment, the first antigen is CD3, in particular human CD3. In an embodiment, the second antigen is CD3, in particular human CD3. In an embodiment, CD3 is bound monovalently by an antigen-binding molecule of the present disclosure.

CD3 is a proven T cell stimulating antigen with therapeutic relevance. Binding to CD3 mimics the T-cell receptor (TCR) leading to T-cell activation. Using CD3 binding molecules in a multispecific antigen-binding molecule in such a way that the target cells and the T-cells are bridged via the multispecific antigen-binding molecule resulting in the formation of an immunological synapse, the effector T cells are able to kill the target cell directly. It is known that efficacy and safety of such molecules with co-engagement of CD3 is mainly driven by the binding valency, the affinity of both specificities and the format used. The binding format should engage CD3 monovalently with moderate to low binding affinity to reduce the potential risk for side effects as discussed before. In order to increase the efficacy without the need for increasing affinity, the epitope of the target antigen (e.g. the first antigen) and of CD3 (e.g. the second antigen) should be in close proximity to enable the immunological synapse (Bluemel C., Cancer Immunol. Immunother. 2010 August; 59(8):1197-209). In addition, the format should further supports low frequencies of dosage in such a way that a usual IgG pharmacokinetic is achieved e.g. via an Fc region.

In an embodiment of the present disclosure, the second antigen is CD3, particularly human or cynomolgus CD3, most particularly human CD3. In an embodiment, the second antigen is the epsilon subunit of CD3. In an embodiment, the second antigen is the epsilon subunit of CD3 comprising SEQ ID NO: 1.

In an embodiment, the second Fv region of an antigen-binding molecule according to the present disclosure specifically binds to CD3, particularly human or cynomolgus CD3, most preferably human CD3. In an embodiment, CD3 is bound monovalently by an antigen-binding molecule according to the present disclosure. In an embodiment, the second Fv region can compete with a monoclonal antibody specific for CD3 for binding to an epitope of CD3. In an embodiment, the second Fv region can compete with any one of the antibodies specific for CD3 as described in Tables 3-5 for binding to an epitope of CD3.

In an embodiment, the second Fv region present in an antigen-binding molecule according to the present disclosure can compete with the monoclonal antibody comprising the VH of SEQ ID NO: 4 and the VL of SEQ ID NO: 5 for binding to an epitope of CD3.

In an embodiment, the second Fv region can compete with the monoclonal antibody comprising the VH of SEQ ID NO: 2 and the VL of SEQ ID NO: 3 for binding to an epitope of CD3.

In an embodiment, the second Fv region can compete with the monoclonal antibody comprising the VH of SEQ ID NO: 6 and the VL of SEQ ID NO: 7 for binding an epitope of CD3.

In a further embodiment, the second Fv region specific for CD3 comprises a VH that is at least 80%, 85%, 90%, 95%, 96%, 97%, 98%, 99% or 100% identical to SEQ ID SEQ ID NO: 2 and a VL sequence that is at least 80%, 85%, 90%, 95%, 96%, 97%, 98%, 99% or 100% identical to SEQ ID NO: 3 or variants thereof that retain functionality.

In a further embodiment, the second Fv region that is specific for CD3 comprises a VH sequence that is at least 80%, 85%, 90%, 95%, 96%, 97%, 98%, 99% or 100% identical to SEQ ID SEQ ID NO: 4 and a VL sequence that is at least 80%, 85%, 90%, 95%, 96%, 97%, 98%, 99% or 100% identical to SEQ ID NO: 5 or variants thereof that retain functionality.

In a further embodiment, the second Fv region that is specific for CD3 comprises a VH sequence that is at least 80%, 85%, 90%, 95%, 96%, 97%, 98%, 99% or 100% identical to SEQ ID SEQ ID NO: 6 and a VL sequence that is at least 80%, 85%, 90%, 95%, 96%, 97%, 98%, 99% or 100% identical to SEQ ID NO: 7 or variants thereof that retain functionality.

In an embodiment of to the present disclosure, an antigen-binding molecule according to the present disclosure is capable of simultaneous binding to a target cell antigen, particularly a tumor-associated antigen expressed on a cancer cell and CD3 expressed on an immune effector cell. In one such embodiment, the target antigen is bound bivalently and CD3 is bound monovalently.

In an embodiment, an antigen-binding molecule according to the present disclosure is capable of crosslinking a T-cell and a target cell by simultaneous binding to a target cell antigen and CD3. In an embodiment, such simultaneous binding results in lysis of the target cell, particularly lysis of a tumor cell. In one embodiment, such simultaneous binding results in activation of the T-cell. In an embodiment, the simultaneous binding results in a cellular response of a T-lymphocyte, particularly a cytotoxic T-lymphocyte, selected from the group of: proliferation, differentiation, cytokine secretion, cytotoxic effector molecule release, cytotoxic activity, and expression of activation markers. In an embodiment, binding of an antigen-binding molecule according to the present disclosure to CD3 without simultaneous binding to the target cell antigen does not result in T-cell activation. In an embodiment, the antigen-binding molecule is capable of re-directing cytotoxic activity of a T-cell to a target cell. In a particular embodiment, the re-direction is independent of MHC-mediated peptide antigen presentation by the target cell and and/or specificity of the T-cell. Particularly, a T-cell according to any of the embodiments according to the present disclosure is a cytotoxic T-cell. In some embodiments, the T-cell is a CD4+ or a CD8+ T cell, particularly a CD8+ T cell.

A format realized in the antigen-binding molecules of the present disclosure enables bivalent binding to a first antigen and monovalent binding to a second antigen and as such combines high affinity binding and avidity effects for the first antigen resulting in a significant difference in binding affinity between CD3 and the target antigen.

An antigen-binding molecule according to the present disclosure are particularly beneficial for targeting different antigens. However, in some cases it may be beneficial to target only one antigen and as such have specificity for the same antigen.

### Nucleic Acids

The present disclosure provides a nucleic acid composition comprising a nucleic acid sequence or a plurality of nucleic acid sequences encoding an antigen-binding molecule according to the present disclosure. An antigen-binding molecule according to the present disclosure may consist of one, two, three, four, or even more polypeptides. Each of said polypeptides may be encoded by the same or by different nucleic acid sequences. Likewise, the nucleic acid sequences encoding said individual polypeptides of an antigen-binding molecule according to the present invention may be present on the same or on different vectors.

In an embodiment, the present disclosure provides a nucleic acid composition comprising a nucleic acid sequence or a plurality of nucleic acid sequences encoding an antigen-binding molecule according to the present disclosure. In an embodiment, the present disclosure provides a nucleic acid composition comprising a nucleic acid sequence or a plurality of nucleic acid sequences encoding any of the antigen-binding molecules described in Tables 9-13. In an embodiment, the nucleic acid composition is an isolated nucleic acid composition.

In an embodiment, a first nucleic acid sequence encodes a polypeptide comprising from the N-terminus to the C-terminus the heavy or light chain of the first Fab, the VH or VL of the second Fv region and the first Fc region subunit. In one such embodiment, a second nucleic acid encodes a polypeptide comprising the complementary heavy or light chain of the first Fab. In one such embodiment, a third nucleic acid encodes a polypeptide comprising from the N-terminus to the C-terminus the complementary VH or VL of the second Fv region and the second Fc region subunit. In an alternative embodiment, a third nucleic acid encodes a polypeptide comprising from the N-terminus to the C-terminus the heavy or light chain of the second Fab, the complementary VH or VL of the second Fv region and the second Fc region subunit. In one such embodiment, a fourth nucleic acid sequence encodes a polypeptide comprising the complementary light or heavy chain of the second Fab.

The polypeptides encoded by the nucleic acid sequence or the plurality of nucleic acid sequences may associate after expression through, e.g., disulfide bonds or other means to form a functional antigen-binding molecule as described herein. For example, the light chain of the first Fab may be encoded by a separate nucleic acid sequence than the portion of an antigen-binding molecule comprising the heavy chain of the first Fab. When co-expressed, the light chain of the first Fab will associate with the heavy chain of the first Fab to form the first Fab comprising a first Fv region. In another example, the portion of an antigen-binding molecule comprising the first Fc region subunit could be encoded by a separate nucleic acid sequence than the portion of an antigen binding-molecule comprising the second Fc region subunit. When co-expressed, the two Fc region subunits will associate to form the dimeric Fc region of an antigen-binding molecule according to the present disclosure.

In an embodiment, the present disclosure is directed to a nucleic acid sequence or a plurality of nucleic acid sequences encoding an antigen-binding molecule according to the present disclosure, wherein the nucleic acid sequence or the plurality of nucleic acid sequences encodes for the individual polypeptides of the antigen-binding molecule. Polypeptides forming the exemplified antigen-binding molecules according to the present disclosure are described in Tables 9-13.

### Vector

In an embodiment, the present disclosure provides a vector composition comprising a vector or a plurality of vectors comprising a nucleic acid sequence composition according to the present disclosure. In an embodiment, the present disclosure provides a vector composition comprising a vector or a plurality of vectors comprising a nucleic acid sequence or plurality of nucleic acid sequences encoding an antigen-binding molecule according to the present disclosure. In an embodiment, the present disclosure provides a vector composition comprising a vector or a plurality of vectors comprising a nucleic acid sequence or plurality of nucleic acid sequences encoding an antigen-binding molecule as described in Tables 9-13. In certain embodiments, the vector is an expression vector.

### Host Cell

In certain embodiments, the present disclosure provides a host cell comprising a vector composition comprising a vector or a plurality of vectors comprising a nucleic acid composition comprising a nucleic acid sequence or plurality of nucleic acid sequences encoding an antigen-binding molecule according to the present disclosure. In an embodiment, the present disclosure refers to a host cell comprising a vector composition comprising a vector or a plurality of vectors comprising a nucleic acid composition comprising the nucleic acid sequence or plurality of nucleic acid sequences encoding an antigen-binding molecule as described in Tables 9-13.

Host cells suitable for replicating and for supporting expression of an antigen-binding molecule according to the present disclosure are well known in the art. Such host cells may be transfected or transduced as appropriate with the particular expression vector(s) and large quantities of vector containing cells can be grown for seeding large scale fermenters to obtain sufficient quantities of such an antigen-binding molecule for clinical applications. Standard technologies are known in the art to express foreign genes in these systems. In general, such steps typically include transforming or transfecting a suitable host cell with a nucleic acid composition or a vector composition, which encodes the individual polypeptides of an antigen-binding molecule according to the present disclosure. Further, such steps typically include culturing the host cells under conditions suitable for the proliferation (multiplication, growth) of the host cells and a culturing step under conditions suitable for the production (expression, synthesis) of the encoded polypeptides.

### Production

Methods to produce antibodies or antigen-binding molecules as disclosed herein are well known in the art (see e.g. Harlow and Lane, “Antibodies, a laboratory manual”, Cold Spring Harbor Laboratory, 1988). An antigen-binding molecule according to the present disclosure may be obtained, for example, by solid-state peptide synthesis or recombinant production. For recombinant production, one or more nucleic acid sequences encoding an antigen-binding molecule according to the present disclosure are isolated and inserted into one or more vectors for further cloning and/or expression in a host cell.

Methods which are well known to those skilled in the art can be used to construct expression vectors containing the coding sequences for an antigen-binding molecule according to the present disclosure along with appropriate transcriptional/translational control signals. Such methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al, CURRENT PROTOCOLS IN MOLECULAR BIOLOGY, Greene Publishing Associates and Wiley Interscience, N.Y (1989). The vectors can be introduced into the appropriate host cells such as prokaryotic (e.g., bacterial) or eukaryotic (e.g., yeast or mammalian) cells by methods well known in the art (see, e.g., “Current Protocol in Molecular Biology”, Ausubel et al. (eds.), Greene Publishing Assoc. and John Wiley Interscience, New York, 1989 and 1992). Numerous cloning vectors are known to those of skill in the art, and the selection of an appropriate cloning vector is a matter of choice. The coding sequences can be placed under the control of a promoter, ribosome binding site (for bacterial expression) and, optionally, an operator, so that the DNA sequence encoding the desired protein or polypeptide is transcribed into RNA in the host cell transformed by a vector or vectors containing this expression construct. The coding sequence may or may not contain a signal peptide or leader sequence. Depending on the expression system and host cell selected, an antigen-binding molecule according to the present disclosure is produced by growing host cells transformed by expression vectors described before under conditions whereby the protein of interest is expressed. The protein is then isolated from the host cells and purified. If the expression system secretes the protein into growth media, the protein can be purified directly from the media. If the protein is not secreted, it is isolated from cell lysates or recovered from the cell membrane fraction. The selection of the appropriate growth conditions and recovery methods are within the skill of the art.

It should be noted that an antigen-binding molecule according to the present disclosure is not a naturally occurring protein. Typically, an antigen-binding molecule according to the present disclosure is a recombinant, synthetic or semi-synthetic protein.

In an embodiment, a method of producing a antigen-binding molecule according to the present disclosure is provided, wherein the method comprises culturing a host cell comprising vector composition comprising a vector or a plurality of vectors comprising a nucleic acid sequence or plurality of nucleic acid sequences encoding an antigen-binding molecule according to the present disclosure, under conditions suitable for expression of an antigen-binding molecule, and recovering an antigen-binding molecule from the host cell or host cell culture medium.

In embodiments, the methods for the production of antigen-binding molecules according to the present disclosure further comprise the step of isolating the produced antigen-binding molecules from the host cells or medium. An antigen-binding molecule recovered as described herein may be purified techniques know in the art, such as high performance liquid chromatography (HPLC), ion exchange chromatography, gel electrophoresis, affinity chromatography, size exclusion chromatography, and the like. The conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which an antigen-binding molecule binds. For example, for affinity chromatography purification of antigen-binding molecules according to the present disclosure, a matrix with protein A or protein G may be used. The purity of an antigen-binding molecule can be determined by any of a variety of well-known analytical methods including gel electrophoresis, high-pressure liquid chromatography, and the like.

### Fusion Proteins

An antigen-binding molecule according to the present disclosure may or may not be fused to one or more other moieties. Such a fusion protein may be prepared in any suitable manner, including genetically or chemically approaches. Said linked moieties may contain secretory or leader sequences, sequences that aid detection, expression, separation or purification, or sequences that confer to increased protein stability, for example, during recombinant production. Non-limiting examples of potential moieties include beta-galactosidase, glutathione-S-transferase, luciferase, a T7 polymerase fragment, a secretion signal peptide, an antibody or antibody fragment, a toxin, a reporter enzyme, a moiety being capable of binding a metal ion like a poly-histidine tag, a tag suitable for detection and/or purification, a homo- or heteroassociation domain, a moiety which increases solubility of a protein, or a moiety which comprises an enzymatic cleavage site. Accordingly, an antigen-binding molecule according to the present disclosure may optionally contain one or more moieties for binding to other targets or target proteins of interest. It should be clear that such further moieties may or may not provide further functionality to an antigen-binding molecule according to the present disclosure and may or may not modify the properties of an antigen-binding molecule according to the present disclosure. The polypeptides according to the present disclosure may be fused by linkers as defined herein.

### Functionality

An antigen-binding molecule according to the present disclosure may be used for the prevention and treatment of diseases, which are mediated by biological pathways in which a target antigen of interest is involved. This may be achieved for instance by inhibiting the interaction between a target antigen and its cognate receptor or natural binding partner. The biological activity of an antigen-binding molecule according to the present disclosure can be measured by various assays known in the art, including those described in Examples 3-4 disclosed herein. Methods for assaying functional activity may utilize binding assays, such as the enzyme-linked immunosorbent assay (ELISA), radioimmunoassay (RIA), fluorescence activated cell sorting (FACS) and other methods that are well known in the art (see Hampton, R. et al. (1990; Serological Methods a Laboratory Manual, APS Press, St Paul, Minn.) and Maddox, D. E. et al. (1983; J. Exp. Med. 158:1211-1216). Alternatively, assays may test the ability of an antigen-binding molecule in eliciting a biological response because of binding to a biological target antigen, either in vivo or in vitro. Biological activities may for example include the induction of proliferation of T cells, the induction of signaling in T cells, the induction of expression of activation markers in T cells, the induction of cytokine secretion by T cells, the inhibition of signaling in target cells such as tumor cells or cells of the tumor stroma, the inhibition of proliferation of target cells, the induction of lysis of target cells, and the induction of tumor regression and/or the improvement of survival.

In an embodiment, the present disclosure provides a method for inducing lysis of a cancer cell, comprising contacting said cancer target cell in the presence of a cytotoxic T-cell with an antigen-binding molecule according to the present disclosure.

In an embodiment, the present disclosure provides a method for inhibition of signaling in cancer cells comprising contacting said cancer cells in the presence of a cytotoxic T-cell with an antigen-binding molecule according to the present disclosure.

In an embodiment, the present disclosure provides a method for inhibition of proliferation of cancer cells, comprising contacting said cancer cells in the presence of a cytotoxic T-cell with an antigen-binding molecule according to the present disclosure.

In an embodiment, the present disclosure provides a method for killing a cancer antigen high expressing cells but not cancer antigen low expressing cells, comprising contacting said cancer antigen high expressing cells in the presence of a cytotoxic T-cell with an antigen-binding molecule according to the present disclosure.

In an embodiment, the present disclosure provides a method for inducing a cellular response in cytotoxic T-cells, comprising contacting said cytotoxic T-cell in the presence of a cancer cell with an antigen-binding molecule according to the present disclosure. In an embodiment, said cellular response is selected from the group consisting of: proliferation, differentiation, cytokine secretion, cytotoxic effector molecule release, cytotoxic activity, and expression of activation markers.

In an embodiment, the present disclosure provides a method for inducing human T-cell proliferation in the presence of cancer cells, comprising contacting said cancer cell in the presence of a T-cell with a antigen-binding molecule according to the present disclosure

In an embodiment, the present disclosure provides a method for stimulating a primary T-cell response in the presence of cancer cells, comprising contacting said cancer cells in the presence of said T-cell with an antigen-binding molecule according to the present disclosure.

In an embodiment, the present disclosure provides a method for re-directing cytotoxic activity of a T-cell to a cancer cell, comprising contacting said cancer cells in the presence of said T-cell with an antigen-binding molecule according to the present disclosure.

In an embodiment, the present disclosure provides the use of an antigen-binding molecule according to the present disclosure for the treatment of cancer that is positive for a cancer associated antigen in a subject, comprising:
(a) selecting a subject who is afflicted with a cancer,
(b) collecting one or more biological samples from the subject,
(c) identifying the cancer associated antigen expressing cancer cells in the one or more samples; and
(d) administering to the subject an effective amount of an antigen-binding molecule according to the present disclosure.

### Diagnostics

In an embodiment, the present disclosure provides the use of an antigen-binding molecule according to the present disclosure for the diagnosis of a disease. In an embodiment, the present disclosure provides the use of an antigen-binding molecule according to the present disclosure for the detection of an antigen. In an embodiment, the present disclosure provides a method for detecting an antigen in a subject or a sample, comprising the step of contacting said subject or sample with an antigen-binding molecule according to the present disclosure. In an embodiment, the present disclosure provides a method for diagnosing a disease in a subject, comprising the step of contacting said subject or sample with an antigen-binding molecule according to the present disclosure.

### Therapeutic Methods

An antigen-binding molecule according to the present disclosure may be used in therapeutic methods. An antigen-binding molecule according to the present disclosure may be used for the treatment of cancer. In an embodiment, the present disclosure provides a method for the treatment of a disease. In an embodiment, the present disclosure provides an antigen-binding molecule according to the present disclosure for the treatment of a disease. In an embodiment, the present disclosure provides an antigen-binding molecule according to the present disclosure for use in the treatment of a disease. In an embodiment, the present disclosure provides an antigen-binding molecule according to the present disclosure for use in the treatment of a disease in an individual in need thereof. In an embodiment, the present disclosure provides the use of an antigen-binding molecule according to the present disclosure for the manufacture of a medicament. In an embodiment, the present disclosure provides an antigen-binding molecule according to the present disclosure for use as a medicament. In an embodiment, the present disclosure provides an antigen-binding molecule according to the present disclosure for use as a medicament for the treatment of a disease in an individual in need thereof. In an embodiment, the disease is associated with the undesired presence of an antigen. In an embodiment, the disease to be treated is a proliferative disease. In a particular embodiment, the disease is cancer.

Non-limiting examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer.

In an embodiment, the present disclosure provides an antigen-binding molecule according to the present disclosure for use in a method of treating a subject or individual having a disease comprising administering to the subject a therapeutically effective amount of an antigen-binding molecule according to the present disclosure. In an embodiment, the method further comprises administering to the individual a therapeutically effective amount of at least one additional therapeutic agent. The subject or individual in need of treatment is typically a mammal, more specifically a human. For use in therapeutic methods, an antigen-binding molecule according to the present disclosure would be formulated, dosed, and administered in a way consistent with good medical practice.

In an embodiment, the present disclosure provides a method for induction of tumor regression in a patient who has cancer, comprising administering to said subject, a therapeutically effective amount of an antigen-binding molecule according to the present disclosure.

In an embodiment, the present disclosure provides a method for improving survival of a subject who has cancer, comprising administering to said subject, a therapeutically effective amount of an antigen-binding molecule according to the present disclosure.

In an embodiment, the present disclosure provides a method for eliciting, stimulating or inducing an immune response in a subject who has cancer, comprising administering to said subject, a therapeutically effective amount of an antigen-binding molecule according to the present disclosure.

In an embodiment, the present disclosure provides a method for enhancing or inducing anti-cancer immunity in a subject who has cancer, comprising administering to said subject, a therapeutically effective amount of an antigen-binding molecule according to the present disclosure.

### Pharmaceutical Compositions

In an embodiment, the present disclosure provides a pharmaceutical composition comprising an antigen-binding molecules according to the present disclosure and at least one pharmaceutically acceptable carrier. The pharmaceutical compositions may further comprise at least one other pharmaceutically active compound. The pharmaceutical composition according to the present disclosure can be used in the diagnosis, prevention and/or treatment of diseases associated with a target antigen of interest.

In particular, the present disclosure provides a pharmaceutical compositions comprising an antigen-binding molecules according to the present disclosure that is suitable for prophylactic, therapeutic and/or diagnostic use in a mammal, more particular in a human. In general, an antigen-binding molecule according to the present disclosure may be formulated as a pharmaceutical composition comprising at least one antigen-binding molecule according to the present disclosure and at least one pharmaceutically acceptable carrier, diluent or excipient and/or adjuvant, and optionally one or more further pharmaceutically active compounds. Such a formulation may be suitable for oral, parenteral, topical administration or for administration by inhalation.

In particular, an antigen-binding molecule according to the present disclosure may be used in combination with one or more pharmaceutically active compounds that are or can be used for the prevention and/or treatment of the diseases in which a target antigen of interest is involved, as a result of which a synergistic effect may or may not be obtained. Examples of such compounds, as well as routes, methods and pharmaceutical formulations or compositions for administering them will be clear to the clinician. In an embodiment, the present disclosure provides a pharmaceutical composition comprising an antigen-binding molecule according to the present disclosure for use in the prevention and/or treatment of a disease associated with the undesired presence of a target antigen specifically. In an embodiment, the present disclosure provides a pharmaceutical composition comprising an antigen-binding molecule according to the present disclosure for the use as a medicament. In an embodiment, the present disclosure provides a pharmaceutical composition comprising an antigen-binding molecule according to the present disclosure for use in the prevention and/or treatment of autoimmune diseases, inflammatory diseases, cancer, vascular diseases, infectious diseases, thrombosis, myocardial infarction, and/or diabetes.

In an embodiment, the disclosure provides a method for the treatment of autoimmune diseases, inflammatory diseases, cancer, vascular diseases, infectious diseases, thrombosis, myocardial infarction, and/or diabetes in a subject in need thereof using a pharmaceutical composition comprising an antigen-binding molecule according to the present disclosure.

Further provided is a method of producing an antigen-binding molecules according to the present disclosure in a form suitable for administration in vivo, the method comprising (a) obtaining an antigen-binding molecule by a method according to the present disclosure, and (b) formulating said antigen-binding molecule with at least one pharmaceutically acceptable carrier, whereby a preparation of antigen-binding molecule is formulated for administration in vivo.

Pharmaceutical compositions according to the present disclosure comprise a therapeutically effective amount of one or more antigen-binding molecules according to the present disclosure dissolved in a pharmaceutically acceptable carrier.

In an embodiment, the present disclosure provides a kit comprising the antigen-binding molecule according to the present disclosure or a pharmaceutical composition comprising the antigen-binding molecule according to the present disclosure.

In an embodiment, the present disclosure provides a kit comprising the antigen-binding molecule according to the present disclosure or a pharmaceutical composition comprising the antigen-binding molecule according to the present disclosure, and a package insert comprising instructions for administration of a binding molecule according to the present disclosure for treating or delaying progression of cancer or reducing or inhibiting tumor growth in a subject in need thereof.

### Dosage

For the prevention or treatment of a disease, the appropriate dosage of an antigen-binding molecule according to the present disclosure will depend on the type of disease to be treated, the route of administration, the body weight of the individual, the particular type of antigen-binding molecule, the severity and course of the disease, whether the antigen-binding molecule is administered for preventive or therapeutic purposes, previous or concurrent therapeutic interventions, the individual's clinical history and response to of antigen-binding molecule, and the discretion of the attending physician. An antigen-binding molecule according to the present disclosure is suitably administered to the patient at one time or over a series of treatments. Depending on the type and severity of the disease, 1 μg/kg to 15 mg/kg (e.g. 0.1 mg/kg-10 mg/kg) of an antigen-binding molecule according to the present disclosure can be an initial dosage for administration to the individual, whether, for example, by one or more separate administrations, or by continuous infusion. One typical daily dosage might range from 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage for an antigen-binding molecule according to the present disclosure would be in the range from 0.005 mg/kg to 10 mg/kg. In other non-limiting examples, a dose may also comprise 1 μg/kg body weight, 5 μg/kg body weight, 10 μg/kg body weight, 50 μg/kg body weight, 100 μg/kg body weight, 200 μg/kg body weight, 350 μg/kg body weight, 500 μg/kg body weight, 1 mg/kg body weight, 5 mg/kg body weight, 10 mg/kg body weight, 50 mg/kg body weight, 100 mg/kg body weight, 200 mg/kg body weight, 350 mg/kg body weight, 500 mg/kg body weight, to 1000 mg/kg body weight or more per administration, and any range derivable therein. In non-limiting examples of a derivable range from the numbers listed herein, a range of 5 mg/kg body weight to 100 mg/kg body weight, 5 μg/kg body weight to 500 mg/kg body weight, etc., can be administered, based on the numbers described above. Thus, one or more doses of 0.5 mg/kg, 2.0 mg/kg, 5.0 mg/kg or 10 mg/kg (or any combination thereof) may be administered to the individual. Such doses may be administered intermittently, e.g. every week or every three weeks (e.g. such that the individual receives from two to twenty, or e.g. six doses of the antigen-binding molecule). An initial higher loading dose, followed by one or more lower doses may be administered. An antigen-binding molecule according to the present disclosure will generally be used in a therapeutically amount effective to achieve the intended purpose.

### Combination Therapies

An antigen-binding molecule according to the present disclosure may be administered in combination with one or more other therapeutic agents. “Therapeutic agent” encompasses any agent administered to treat a symptom or disease in an individual in need of such treatment. In certain embodiments, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers. Such other therapeutic agents are suitably present in combination in amounts that are effective for the purpose intended. Combination therapies encompass combined administration (where two or more therapeutic agents are included in the same or separate compositions), and separate administration, in which case, administration of an antigen-binding molecule according to the present disclosure can occur prior to, simultaneously, and/or following, administration of the additional therapeutic agent. An antigen-binding molecule according to the present disclosure can also be used in combination with radiation therapy.

### Sequences

TABLE 2
Amino acid sequence of the extracellular domain of human CD3 epsilon

| Target protein | SEQ ID NO: | [aa] |
|---|---|---|
| Mature human CD3 | 1 | DGNEEMGGITQTPYKVSISGTTVILTCPQYPGSEIL |
| epsilon-ECD (1-118) | | WQHNDKNIGGDEDDKNIGSDEDHLSLKEFSELEQS |
| | | GYYVCYPRGSKPEDANFYLYLRARVCENCMEMD |

TABLE 3
VH and VL amino acid sequences of CD3 specific antibody “SP34”

| Antibody | Chain | SEQ ID NO | [aa] |
|---|---|---|---|
| SP34 | VH | 2 | EVQLVESGGGLVQPKGSLKLSCAASGFTFNTYAMN |
| | | | WVRQAPGKGLEWVARIRSKYNNYATYYADSVKDRF |
| | | | TISRDDSQSILYLQMNNLKTEDTAMYYCVRHGNFGN |
| | | | SYVSWFAYWGQGTLVTVSS |
| SP34 | VL | 3 | QAVVTQESALTTSPGETVTLTCRSSTGAVTTSNYAN |
| | | | WVQEKPDHLFTGLIGGTNKRAPGVPARFSGSLIGDK |
| | | | AALTITGAQTEDEAIYFCALWYSNLWVFGGGTKLTVL |
| | | | GQ |

TABLE 4
VH and VL amino acid sequences of CD3 specific antibody “I2C”

| Antibody | Chain | SEQ ID NO: | [aa] |
|---|---|---|---|
| I2C | VH | 4 | EVQLVESGGGLVQPGGSLKLSCAASGFTFNKYAMN |
| | | | WVRQAPGKGLEWVARIRSKYNNYATYYADSVKDRFT |
| | | | ISRDDSKNTAYLQMNNLKTEDTAVYYCVRHGNFGNS |
| | | | YISYWAYWGQGTLVTVSS |
| I2C | VL | 5 | QTVVTQEPSLTVSPGGIVTLICGSSTGAVTSGNYPN |
| | | | WVQQKPGQAPRGLIGGTKFLAPGTPARFSGSLLGGK |
| | | | AALTLSGVQPEDEAEYYCVLWYSNRWVFGGGTKLTV |
| | | | L |

TABLE 5
VH and VL amino acid sequences of CD3 specific antibody “Roche”

| Antibody | Chain | SEQ ID NO: | [aa] |
|---|---|---|---|
| Roche | VH | 6 | EVQLLESGGGLVQPGGSLRLSCAASGFTFSTYAM |
| | | | NWVRQAPGKGLEWVSRIRSKYNNYATYYADSVK |
| | | | GRFTISRDDSKNTLYLQMNSLRAEDTAVYYCVRH |
| | | | GNFGNSYVSWFAYWGQGTLVTVSS |
| Roche | VL | 7 | QAVVTQEPSLTVSPGGTVTLTCGSSTGAVTTSNY |
| | | | ANWVQEKPGQAFRGLIGGTNKRAPGTPARFSGS |
| | | | LLGGKAALTLSGAQPEDEAEYYCALVVYSNLWVFG |
| | | | GGTKLTVLGQ |

TABLE 6
VH and VL amino acid sequence of HER2 specific antibody Trastuzumab

| Antibody | Chain | SEQ ID NO: | [aa] |
|---|---|---|---|
| Trastuzumab | VH | 8 | QVQLVESGGGLVQPGGSLRLSCAASGFNIKDTY |
| | | | IHWVRQAPGKGLEWVARIYPTNGYTRYADSVKG |
| | | | RFTISADTSKNTAYLQMNSLRAEDTAVYYCSRW |
| | | | GGDGFYAMDYWGQGTLVTVSS |
| Trastuzumab | VL | 9 | DIQMTQSPSSLSASVGDRVTITCRASQDVNTAV |
| | | | AWYQQKPGKAPKLLIYSASFLYSGVPSRFSGSR |
| | | | SGTDFTLTISSLQPEDFATYYCQQHYTTPPTFGQ |
| | | | GTKVEIK |

TABLE 7
Amino acid sequences of peptide linkers

| | SEQ ID | |
|---|---|---|
| Linker | NO: | [aa] |
| (GGS)₃ Linker | 10 | GGSGGSGGS |
| CH1_{trunc.} Linker | 11 | ASTKGP |
| CLλ_{trunc.} Linker | 12 | QPKAAP |
| Hinge_{trunc} Linker | 13 | DKTHTCPPCP |
| Combined (CH1_{trunc.} + | 14 | ASTKGPDKTHTCPPCP |
| Hinge_{trunc.}) Linker | | |
| Combined (CLλ_{trunc} + | 15 | QPKAAPDKTHTCPPCP |
| Hinge_{trunc.}) Linker | | |

TABLE 8
Amino acid sequences of heterodimeric Fc region subunits
including N-terminal located IgG hinge derived linker

| Linker + Fc | SEQ | |
|---|---|---|
| region | ID | |
| subunits | NO: | [aa] |
| Linker_{(Hinge)}- | 16 | DKTHTCPPCPAPEAEGAPSVFLFPPKPKDTLMISRTPEVTC |
| CH2_{(AEASS)}- | | VVVDVSHEDPEVKFNWYVDGVEVHNAKTKPREEQYNSTY |
| CH3_{(knob)} | | RVVSVLTVLHQDWLNGKEYKCKVSNKALPSSIEKTISKAKG |
| | | QPREPQVYTLPPCREEMTKNQVSLWCLVKGFYPSDIAVEW |
| | | ESNGQPENNYKTTPPVLDSDGSFFLYSKLTVDKSRWQQG |
| | | NVFSCSVMHEALHNHYTQKSLSLSPGK |
| Linker_{(Hinge)}- | 17 | DKTHTCPPCPAPEAEGAPSVFLFPPKPKDTLMISRTPEVTC |
| CH2_{(AEASS)}- | | VVVDVSHEDPEVKFNWYVDGVEVHNAKTKPREEQYNSTY |
| CH3₍ₕₒₗₑ₎ | | RVVSVLTVLHQDWLNGKEYKCKVSNKALPSSIEKTISKAKG |
| | | QPREPQVCTLPPSREEMTKNQVSLSCAVKGFYPSDIAVEW |
| | | ESNGQPENNYKTTPPVLDSDGSFFLVSKLTVDKSRWQQG |
| | | NVFSCSVMHEALHNHYTQKSLSLSPGK |

TABLE 9
Amino acid sequences of the polypeptides forming a trivalent
bispecific antigen-binding molecule according to the present
disclosure and as shown in FIG. 1B (without a disulfide
stabilized second Fv region) with bivalent binding
to HER2 and monovalent binding to CD3

| Construct 1 | SEQ | |
|---|---|---|
| (Trastuzumab/I2C) | ID NO: | [aa] |
| VH_{(Trast.)}-CH1-Linker_{(GGS)3}- | 18 | QVQLVESGGGLVQPGGSLRLSCAASGFNIKDTY |
| VL_{(12C)}-Linker_{(CLλ+hinge)}- | | IHWVRQAPGKGLEWVARIYPTNGYTRYADSVKG |
| CH2_{(AEASS)}-CH3_{(knob)} | | RFTISADTSKNTAYLQMNSLRAEDTAVYYCSRW |
| | | GGDGFYAMDYWGQGTLVTVSSASTKGPSVFPL |
| | | APSSKSTSGGTAALGCLVKDYFPEPVTVSWNSG |
| | | ALTSGVHTFPAVLQSSGLYSLSSVVTVPSSSLGT |
| | | QTYICNVNHKPSNTKVDKRVEPKSCGGSGGSG |
| | | GSQTVVTQEPSLTVSPGGTVTLTCGSSTGAVTS |
| | | GNYPNWVQQKPGQAPRGLIGGTKFLAPGTPAR |
| | | FSGSLLGGKAALTLSGVQPEDEAEYYCVLWYSN |
| | | RWVFGGGTKLTVLGQPKAAPDKTHTCPPCPAP |
| | | EAEGAPSVFLFPPKPKDTLMISRTPEVTCVVVDV |
| | | SHEDPEVKFNWYVDGVEVHNAKTKPREEQYNS |
| | | TYRVVSVLTVLHQDWLNGKEYKCKVSNKALPSS |
| | | IEKTISKAKGQPREPQVYTLPPCREEMTKNQVSL |
| | | WCLVKGFYPSDIAVEWESNGQPENNYKTTPPVL |
| | | DSDGSFFLYSKLTVDKSRWQQGNVFSCSVMHE |
| | | ALHNHYTQKSLSLSPGK |
| VH_{(Trast.)}-CH1-Linker_{(GGS)3}- | 19 | QVQLVESGGGLVQPGGSLRLSCAASGFNIKDTY |
| VH_{(12C)}-Linker_{(CH1+hinge)}- | | IHWVRQAPGKGLEWVARIYPTNGYTRYADSVKG |
| CH2_{(AEASS)}-CH3₍ₕₒₗₑ₎ | | RFTISADTSKNTAYLQMNSLRAEDTAVYYCSRW |
| | | GGDGFYAMDYWGQGTLVTVSSASTKGPSVFPL |
| | | APSSKSTSGGTAALGCLVKDYFPEPVTVSWNSG |
| | | ALTSGVHTFPAVLQSSGLYSLSSVVTVPSSSLGT |
| | | QTYICNVNHKPSNTKVDKRVEPKSCGGSGGSG |
| | | GSEVQLVESGGGLVQPGGSLKLSCAASGFTFNK |
| | | YAMNWVRQAPGKGLEWVARIRSKYNNYATYYA |
| | | DSVKDRFTISRDDSKNTAYLQMNNLKTEDTAVY |
| | | YCVRHGNFGNSYISYWAYWGQGTLVTVSSAST |
| | | KGPDKTHTCPPCPAPEAEGAPSVFLFPPKPKDT |
| | | LMISRTPEVTCVVVDVSHEDPEVKFNWYVDGVE |
| | | VHNAKTKPREEQYNSTYRVVSVLTVLHQDWLN |
| | | GKEYKCKVSNKALPSSIEKTISKAKGQPREPQVC |
| | | TLPPSREEMTKNQVSLSCAVKGFYPSDIAVEWE |
| | | SNGQPENNYKTTPPVLDSDGSFFLVSKLTVDKS |
| | | RWQQGNVFSCSVMHEALHNHYTQKSLSLSPGK |
| VL_{(Trast )}-CL | 20 | DIQMTQSPSSLSASVGDRVTITCRASQDVNTAV |
| human IgG kappa | | AWYQQKPGKAPKLLIYSASFLYSGVPSRFSGSR |
| | | SGTDFTLTISSLQPEDFATYYCQQHYTTPPTFGQ |
| | | GTKVEIKRTVAAPSVFIFPPSDEQLKSGTASVVC |
| | | LLNNFYPREAKVQWKVDNALQSGNSQESVTEQ |
| | | DSKDSTYSLSSTLTLSKADYEKHKVYACEVTHQ |
| | | GLSSPVTKSFNRGEC |

TABLE 10
Amino acid sequences of the polypeptides forming a trivalent
bispecific antigen-binding molecule according to the present
disclosure and as shown in FIG. 1B (with a disulfide stabilized
second Fv region) with bivalent binding to HER2 and
monovalent binding to CD3

| Construct 2 | SEQ | |
|---|---|---|
| (Trastuzumab/I2C) | ID NO: | [aa] |
| VH_{(Trast.)}-CH1-Linker_{(GGS)3}- | 21 | QVQLVESGGGLVQPGGSLRLSCAASGFNIK |
| VL(G100C)_{(I2C)}-Linker_{(CLλ+hinge)}- | | DTYIHWVRQAPGKGLEWVARIYPTNGYTRY |
| CH2_{(AEASS)}-CH3_{(knob)} | | ADSVKGRFTISADTSKNTAYLQMNSLRAEDT |
| | | AVYYCSRWGGDGFYAMDYWGQGTLVTVS |
| | | SASTKGPSVFPLAPSSKSTSGGTAALGOLVK |
| | | DYFPEPVTVSWNSGALTSGVHTFPAVLQSS |
| | | GLYSLSSVVTVPSSSLGTQTYICNVNHKPSN |
| | | TKVDKRVEPKSCGGSGGSGGSQTVVTQEP |
| | | SLTVSPGGIVTLTCGSSTGAVISGNYPNWV |
| | | QQKPGQAPRGLIGGTKFLAPGTPARFSGSL |
| | | LGGKAALTLSGVQPEDEAEYYCVLVVYSNR |
| | | WVFGCGTKLTVLGQQPKAAPDKTHTCPPCP |
| | | APEAEGAPSVFLFPPKPKDTLMISRTPEVTC |
| | | VVVDVSHEDPEVKFNVVYVDGVEVHNAKTK |
| | | PREEQYNSTYRVVSVLTVLHQDWLNGKEYK |
| | | CKVSNKALPSSIEKTISKAKGQPREPQVYTL |
| | | PPCREEMTKNQVSLWCLVKGFYPSDIAVEW |
| | | ESNGQPENNYKTTPPVLDSDGSFFLYSKLT |
| | | VDKSRWQQGNVFSCSVMHEALHNHYTQKS |
| | | LSLSPGK |
| VH_{(Trast.)}-CH1-Linker_{(GGS)3}- | 22 | QVQLVESGGGLVQPGGSLRLSCAASGFNIK |
| VH(G44C)_{(I2C)}-Linker_{(CH1+hinge)}- | | DTYIHWVRQAPGKGLEWVARIYPTNGYTRY |
| CH2_{(AEASS)}-CH3₍ₕₒₗₑ₎ | | ADSVKGRFTISADTSKNTAYLQMNSLRAEDT |
| | | AVYYCSRWGGDGFYAMDYWGQGTLVTVS |
| | | SASTKGPSVFPLAPSSKSTSGGTAALGCLVK |
| | | DYFPEPVTVSWNSGALTSGVHTFPAVLQSS |
| | | GLYSLSSVVTVPSSSLGTQTYICNVNHKPSN |
| | | TKVDKRVEPKSCGGSGGSGGSEVQLVESG |
| | | GGLVQPGGSLKLSCAASGFTFNKYAMNWV |
| | | RQAPGKCLEWVARIRSKYNNYATYYADSVK |
| | | DRFTISRDDSKNTAYLQMNNLKTEDTAVYYC |
| | | VRHGNFGNSYISYWAYWGQGTLVTVSSAST |
| | | KGPDKTHTCPPCPAPEAEGAPSVFLFPPKP |
| | | KDTLMISRTPEVTCVVVDVSHEDPEVKFNW |
| | | YVDGVEVHNAKTKPREEQYNSTYRVVSVLT |
| | | VLHQDWLNGKEYKCKVSNKALPSSIEKTISK |
| | | AKGQPREPQVCTLPPSREEMTKNQVSLSCA |
| | | VKGFYPSDIAVEWESNGQPENNYKTTPPVL |
| | | DSDGSFFLVSKLTVDKSRWQQGNVFSCSV |
| | | MHEALHNHYTQKSLSLSPGK |
| VL_{(Trast.)}-CL | 23 | DIQMTQSPSSLSASVGDRVTITCRASQDVNT |
| human IgG kappa | | AVAWYQQKPGKAPKLLIYSASFLYSGVPSRF |
| | | SGSRSGTDFTLTISSLQPEDFATYYCQQHYT |
| | | TPPTFGQGTKVEIKRTVAAPSVFIFPPSDEQL |
| | | KSGTASVVCLLNNFYPREAKVQWKVDNALQ |
| | | SGNSQESVTEQDSKDSTYSLSSTLTLSKAD |
| | | YEKHKVYACEVTHQGLSSPVTKSFNRGEC |

TABLE 11
Amino acid sequences of the polypeptides forming a trivalent
bispecific antigen-binding molecule as shown in FIG. 1B
(without a disulfide stabilized second Fv region) with
bivalent binding to HER2 and monovalent binding to CD3

| Contruct 3 | SEQ | |
|---|---|---|
| (Trastuzumab/Roche) | ID NO: | [aa] |
| VH_{(Trast.)}-CH1-Linker_{(GGS)3}- | 24 | QVQLVESGGGLVQPGGSLRLSCAASGFNIK |
| VL_{(Roche)}-Linker_{(CLλ+hinge)}- | | DTYIHWVRQAPGKGLEWVARIYPTNGYTRY |
| CH2_{(AEASS)}-CH3_{(knob)} | | ADSVKGRFTISADTSKNTAYLQMNSLRAEDT |
| | | AVYYCSRWGGDGFYAMDYWGQGTLVTVS |
| | | SASTKGPSVFPLAPSSKSTSGGTAALGCLVK |
| | | DYFPEPVTVSWNSGALTSGVHTFPAVLQSS |
| | | GLYSLSSVVTVPSSSLGTQTYICNVNHKPSN |
| | | TKVDKRVEPKSCGGSGGSGGSQAVVTQEP |
| | | SLTVSPGGTVTLTCGSSTGAVTTSNYANWV |
| | | QEKPGQAFRGLIGGTNKRAPGTPARFSGSL |
| | | LGGKAALTLSGAQPEDEAEYYCALWYSNLW |
| | | VFGGGTKLTVLGQPKAAPDKTHTCPPCPAP |
| | | EAEGAPSVFLFPPKPKDTLMISRTPEVTCVV |
| | | VDVSHEDPEVKFNWYVDGVEVHNAKTKPR |
| | | EEQYNSTYRVVSVLTVLHQDWLNGKEYKCK |
| | | VSNKALPSSIEKTISKAKGQPREPQVYTLPP |
| | | CREEMTKNQVSLWCLVKGFYPSDIAVEWES |
| | | NGQPENNYKTTPPVLDSDGSFFLYSKLTVD |
| | | KSRWQQGNVFSCSVMHEALHNHYTQKSLS |
| | | LSPGK |
| VH_{(Trast.)}-CH1-Linker_{(GGS)3}- | 25 | QVQLVESGGGLVQPGGSLRLSCAASGFNIK |
| VH_{(Roche)}-Linker_{(CH1+hinge)}- | | DTYIHWVRQAPGKGLEWVARIYPTNGYTRY |
| CH2_{(AEASS)}-CH3₍ₕₒₗₑ₎ | | ADSVKGRFTISADTSKNTAYLQMNSLRAEDT |
| | | AVYYCSRWGGDGFYAMDYWGQGTLVTVS |
| | | SASTKGPSVFPLAPSSKSTSGGTAALGCLVK |
| | | DYFPEPVTVSWNSGALTSGVHTFPAVLQSS |
| | | GLYSLSSVVTVPSSSLGTQTYICNVNHKPSN |
| | | TKVDKRVEPKSCGGSGGSGGSEVQLLESG |
| | | GGLVQPGGSLRLSCAASGFTFSTYAMNWV |
| | | RQAPGKGLEWVSRIRSKYNNYATYYADSVK |
| | | GRFTISRDDSKNTLYLQMNSLRAEDTAVYYC |
| | | VRHGNFGNSYVSWFAYWGQGTLVTVSSAS |
| | | TKGPDKTHTCPPCPAPEAEGAPSVFLFPPK |
| | | PKIDTLMISRTPEVTCVVVDVSHEDPEVKFN |
| | | WYVDGVEVHNAKTKPREEQYNSTYRVVSVL |
| | | TVLHQDWLNGKEYKCKVSNKALPSSIEKTIS |
| | | KAKGQPREPQVCTLPPSREEMTKNQVSLSC |
| | | AVKGFYPSDIAVEWESNGQPENNYKTTPPV |
| | | LDSDGSFFLVSKLTVDKSRWQQGNVFSCSV |
| | | MHEALHNHYTQKSLSLSPGK |
| VL_{(Trast.)}-CL | 26 | DIQMTQSPSSLSASVGDRVTITCRASQDVNT |
| human IgG kappa | | AVAWYQQKPGKAPKLLIYSASFLYSGVPSRF |
| | | SGSRSGTDFTLTISSLQPEDFATYYCQQHYT |
| | | TPPTFGQGTKVEIKRTVAAPSVFIFPPSDEQL |
| | | KSGTASVVCLLNNFYPREAKVQWKVDNALQ |
| | | SGNSQESVTEQDSKDSTYSLSSTLTLSKAD |
| | | YEKHKVYACEVTHQGLSSPVTKSFNRGEC |

TABLE 12
Amino acid sequences of the polypeptides forming a
trivalent bispecific antigen-binding molecule as shown in
FIG. 1B (without a disulphide stabilized second Fv region)
with bivalent binding to HER2 and monovalent binding to CD3.

| Construct 4 | SEQ | |
|---|---|---|
| (Trastuzumab/SP34) | ID NO: | [aa] |
| VH_{(Trast.)}-CH1-Linker_{(GGS)3}- | 27 | QVQLVESGGGLVQPGGSLRLSCAASGFNIK |
| VL_{(SP34)}-Linker_{(CLλ+hinge)}- | | DTYIHWVRQAPGKGLEWVARIYPTNGYTRY |
| CH2_{(AEASS)}-CH3_{(knob)} | | ADSVKGRFTISADTSKNTAYLQMNSLRAEDT |
| | | AVYYCSRWGGDGFYAMDYWGQGTLVTVS |
| | | SASTKGPSVFPLAPSSKSTSGGTAALGCLVK |
| | | DYFPEPVTVSWNSGALTSGVHTFPAVLQSS |
| | | GLYSLSSVVTVPSSSLGTQTYICNVNHKPSN |
| | | TKVDKRVEPKSCGGSGGSGGSQAVVTQES |
| | | ALTTSPGETVTLTCRSSTGAVTTSNYANWV |
| | | QEKPDHLFTGLIGGTNKRAPGVPARFSGSLI |
| | | GDKAALTITGAQTEDEAIYFCALWYSNLWVF |
| | | GGGTKLTVLGQPKAAPDKTHTCPPCPAPEA |
| | | EGAPSVFLFPPKPKDTLMISRTPEVTCVVVD |
| | | VSHEDPEVKFNWYVDGVEVHNAKTKPREE |
| | | QYNSTYRVVSVLTVLHQDWLNGKEYKCKVS |
| | | NKALPSSIEKTISKAKGQPREPQVYTLPPCR |
| | | EEMTKNQVSLWCLVKGFYPSDIAVEWESNG |
| | | QPENNYKTTPPVLDSDGSFFLYSKLTVDKSR |
| | | WQQGNVFSCSVMHEALHNHYTQKSLSLSP |
| | | GK |
| VH_{(Trast. )}-CH1-Linker_{(GGS)3}- | 28 | QVQLVESGGGLVQPGGSLRLSCAASGFNIK |
| VH_{(SP34)}-Linker_{(CH1+hinge)}- | | DTYIHWVRQAPGKGLEWVARIYPTNGYTRY |
| CH2_{(AEASS)}-CH3₍ₕₒₗₑ₎ | | ADSVKGRFTISADTSKNTAYLQMNSLRAEDT |
| | | AVYYCSRWGGDGFYAMDYWGQGTLVTVS |
| | | SASTKGPSVFPLAPSSKSTSGGTAALGCLVK |
| | | DYFPEPVTVSWNSGALTSGVHTFPAVLQSS |
| | | GLYSLSSVVTVPSSSLGTQTYICNVNHKPSN |
| | | TKVDKRVEPKSCGGSGGSGGSEVQLVESG |
| | | GGLVQPKGSLKLSCAASGFTFNTYAMNWV |
| | | RQAPGKGLEWVARIRSKYNNYATYYADSVK |
| | | DRFTISRDDSQSILYLQMNNLKTEDTAMYYC |
| | | VRHGNFGNSYVSWFAYWGQGTLVTVSSAS |
| | | TKGPDKTHTCPPCPAPEAEGAPSVFLFPPK |
| | | PKDTLMISRTPEVTCVVVDVSHEDPEVKFN |
| | | WYVDGVEVHNAKTKPREEQYNSTYRVVSVL |
| | | TVLHQDWLNGKEYKCKVSNKALPSSIEKTIS |
| | | KAKGQPREPQVCTLPPSREEMTKNQVSLSC |
| | | AVKGFYPSDIAVEWESNGQPENNYKTTPPV |
| | | LDSDGSFFLVSKLTVDKSRWQQGNVFSCSV |
| | | MHEALHNHYTQKSLSLSPGK |
| VL_{(Trast.)}-CL | 29 | DIQMTQSPSSLSASVGDRVTITCRASQDVNT |
| human IgG kappa | | AVAWYQQKPGKAPKLLIYSASFLYSGVPSRF |
| | | SGSRSGTDFTLTISSLQPEDFATYYCQQHYT |
| | | TPPTFGQGTKVEIKRTVAAPSVFIFPPSDEQL |
| | | KSGTASVVCLLNNFYPREAKVQWKVDNALQ |
| | | SGNSQESVTEQDSKDSTYSLSSTLTLSKAD |
| | | YEKHKVYACEVTHQGLSSPVTKSFNRGEC |

TABLE 13
Amino acid sequences of the polypeptides forming a
trivalent bispecific antigen-binding molecule as shown in
FIG. 1B (without a disulfide stabilized second Fv region)
with bivalent binding to HER2 and monovalent binding to CD3

| Construct 5 | SEQ | |
|---|---|---|
| (Trastuzumab/Neg. Ctrl.) | ID NO: | [aa] |
| VH_{(Trast.)}-CH1-Linker_{(GGS)3}- | 30 | QVQLVESGGGLVQPGGSLRLSCAASGFNIK |
| VL_{(Neg. Ctrl)}-Linker_{(CLλ+hinge)}- | | DTYIHWVRQAPGKGLEWVARIYPTNGYTRY |
| CH2_{(AEASS)}-CH3_{(knob)} | | ADSVKGRFTISADTSKNTAYLQMNSLRAEDT |
| | | AVYYCSRWGGDGFYAMDYWGQGTLVTVS |
| | | SASTKGPSVFPLAPSSKSTSGGTAALGCLVK |
| | | DYFPEPVTVSWNSGALTSGVHTFPAVLQSS |
| | | GLYSLSSVVTVPSSSLGTQTYICNVNHKPSN |
| | | TKVDKRVEPKSCGGSGGSGGSDIELTQPPS |
| | | VSVAPGQTARISCSGDNLPAYTVIWYQQKP |
| | | GQAPVLVIYDDSDRPSGIPERFSGSNSGNTA |
| | | TLTISGTQAEDEADYYCASWDPSSGVVFGG |
| | | GTKLTVLGQPKAAPDKTHTCPPCPAPEAEG |
| | | APSVFLFPPKPKDTLMISRTPEVTCVVVDVS |
| | | HEDPEVKFNWYVDGVEVHNAKTKPREEQY |
| | | NSTYRVVSVLTVLHQDWLNGKEYKCKVSNK |
| | | ALPSSIEKTISKAKGQPREPQVYTLPPCREE |
| | | MTKNQVSLWCLVKGFYPSDIAVEWESNGQ |
| | | PENNYKTTPPVLDSDGSFFLYSKLTVDKSR |
| | | WQQGNVFSCSVMHEALHNHYTQKSLSLSP |
| | | GK |
| VH_{(Trast.)}-CHI-Linker_{(GGS)3}- | 31 | QVQLVESGGGLVQPGGSLRLSCAASGFNIK |
| VH_{(Neg. Ctrl.)}-Linker_{(CH1+hinge)}- | | DTYIHWVRQAPGKGLEWVARIYPTNGYTRY |
| CH2_{(AEASS)}-CH3₍ₕₒₗₑ₎ | | ADSVKGRFTISADTSKNTAYLQMNSLRAEDT |
| | | AVYYCSRWGGDGFYAMDYWGQGTLVTVS |
| | | SASTKGPSVFPLAPSSKSTSGGTAALGCLVK |
| | | DYFPEPVTVSWNSGALTSGVHTFPAVLQSS |
| | | GLYSLSSVVTVPSSSLGTQTYICNVNHKPSN |
| | | TKVDKRVEPKSCGGSGGSGGSQVQLQQSG |
| | | PGLVKPSQTLSLTCAISGDSVSSNSAAWSWI |
| | | RQSPGRGLEWLGRIYYRSKWYNDYAVSVK |
| | | SRITINPDTSKNQFSLQLNSVTPEDTAVYYC |
| | | ARLDHRYHEDTVYPGMDVWGQGTLVTVSS |
| | | ASTKGPDKTHTCPPCPAPEAEGAPSVFLFP |
| | | PKPKDTLMISRTPEVTCVVVDVSHEDPEVKF |
| | | NWYVDGVEVHNAKTKPREEQYNSTYRVVS |
| | | VLTVLHQDWLNGKEYKCKVSNKALPSSIEKT |
| | | ISKAKGQPREPQVCTLPPSREEMTKNQVSL |
| | | SCAVKGFYPSDIAVEWESNGQPENNYKTTP |
| | | PVLDSDGSFFLVSKLTVDKSRWQQGNVFSC |
| | | SVMHEALHNHYTQKSLSLSPGK |
| VL_{(Trast.)}-CL | 32 | DIQMTQSPSSLSASVGDRVTITCRASQDVNT |
| human IgG kappa | | AVAWYQQKPGKAPKLLIYSASFLYSGVPSRF |
| | | SGSRSGTDFTLTISSLQPEDFATYYCQQHYT |
| | | TPPTFGQGTKVEIKRTVAAPSVFIFPPSDEQL |
| | | KSGTASVVCLLNNFYPREAKVQWKVDNALQ |
| | | SGNSQESVTEQDSKDSTYSLSSTLTLSKAD |
| | | YEKHKVYACEVTHQGLSSPVTKSFNRGEC |

### WORKING EXAMPLES

The following are examples of molecules and methods according to the present disclosure. It is understood that various other embodiments may be practiced, given the general description provided herein.

Standard methods were used to manipulate DNA as described in Sambrook et al., Molecular cloning: A laboratory manual; Cold Spring Harbor Laboratory Press, Cold Spring Harbor, N.Y., 1989. General information regarding the nucleotide sequences of human immunoglobulins light and heavy chains is given in: Kabat, E. A. et al., (1991) Sequences of Proteins of Immunological Interest, 5th ed., NIH Publication No. 91-3242.

### Example 1: Preparation, Production and Characterization of Trivalent Bispecific Antigen Binding Molecules

The antigen-binding molecules as exemplified herein were built from an aglycosylated monoclonal human IgG1 template antibody incorporating an additional Fv region (Fv₂) between the Fc region and the two Fab arms of a regular human IgG1 molecule. A basic structure of such a molecule is provided in FIG. 1B.

The fusion between the two Fab arms and the Fc region was achieved by using two glyine-serine linkers ((GGS)₃) (SEQ ID NO: 10) between the C-terminus of the two Fab heavy chains and the N-terminus of the variable domains (VH₂ and VL₂) of the incorporated additional Fv region. The fusion between the variable domains of the second Fv region (Fv₂) and the two Fc region subunits was achieved by using peptide linkers build from the first 5 amino acid residues of a CU. (QPKAAP (SEQ ID NO: 12)) or CH1 (ASTKGP (SEQ ID NO: 11) constant domain and a portion of a human IgG1 hinge sequence (DKTHTCPPCP (SEQ ID NO: 13). The use of the human IgG1 hinge sequence allowed for a further stabilization of the heterodimeric molecule via the formation of interchain-disulfide bridges between the two employed peptide linkers. The Fc region was modified by introducing mutations into the CH3 domain of each Fc region subunit according to the “knob-into-holes” technology. Thereby, the polypeptide comprising one mutated CH3 domain is forced to heterodimerize with the other polypeptide comprising the other CH3 domain, which is engineered in a complementary manner.

In the below exemplified bispecific trivalent constructs, the two Fabs (Fab₁ and Fab₂) present in the two Fab arms of the antigen-binding molecule, specifically bind to the cancer associated target HER2 in a bivalent manner, whereas the incorporated second Fv region (Fv₂) specifically binds to human CD3 epsilon in a monovalent fashion.

For HER2 binding, nucleotide sequences encoding the VH and VL domains from “Trastuzumab” (HERCEPTIN®) as described by Baselga et al. 1998, Cancer Res 58(13): 2825-2831) were used. Trastuzumab and its method of preparation are described in U.S. Pat. No. 5,821,337.

For CD3 binding, the following nucleotide sequences encoding the VH and VL domains of the following CD3 antibodies were used:
SP34: a monoclonal antibody described by Yoshino et al. (Exp. Anim. 49(2), 97-110, 2000).
I2C: a monoclonal antibody described in WO 2008/119566 (MICROMET AG)
A monoclonal CD3 antibody disclosed in WO 2016/020309 (F. HOFFMANN-LA ROCHE AG) referred herein to as antibody “Roche”.
An in-house negative control antibody with specificity for chicken lysozyme.

A summary of the individual components (Fabs, Linkers, Fv regions, Fc region etc.) of the produced bispecific trivalent antigen-binding molecules according to the present disclosure (referred herein to as Constructs 1-5) made in accordance with the examples described herein are set forth in Tables: 3-13.

### Gene Synthesis

All nucleic acid sequences or desired gene segments were either generated by PCR using appropriate templates or were gene synthesized as linear DNA fragments with appropriate flanking regions (e.g. suitable restriction enzyme recognition sites, linker sequences) in-house or by an external provider. The nucleic acid sequences or gene segments flanked by singular restriction endonuclease cleavage sites were cloned into respective expression vectors (e.g. mammalian IgG expression vectors) or sequencing vectors using standard molecular biology methods. When intended for use in mammalian expression vectors, all constructs were designed with a 5′-end DNA sequence coding for a leader peptide which targets proteins for secretion in eukaryotic cells. The DNA sequence of the subcloned gene fragments was confirmed by DNA by double strand sequencing.

### Production

For expression of Construct 1-5, exponentially growing eukaryotic HEK293 cells were transfected with a mammalian two vector expression system encoding all components of the Constructs, resulting in a 1:1:2 ratio of the two polypeptides comprising the Fc region subunits and the polypeptide comprising the light chain of the first and second Fab, respectively.

Cell culture supernatants were harvested on day 6 post transfection and subjected to standard Protein A affinity chromatography (MabSelect SURE|GE Healthcare). Buffer exchange was performed to 1× Dulbcecco's PBS (pH 7.2|Invitrogen) and samples were sterile filtered (0.2 μm pore size). Protein concentrations were determined by UV-spectrophotometry and purities of the constructs were analyzed under denaturing, reducing and non-reducing conditions using CE-SDS (LabChip GXII|Perkin Elmer|USA). HP-SEC was performed to analyze IgG preparations in native state.

### Production Results

Table 14 summarizes yields and final monomer content of the different preparations obtained for the produced constructs. In general, the constructs could be generated by the described production and purification method with yields between 29-75 mg/L and final monomer content between 65-90%. The use of a stabilizing disulfide bridge (VH-G44C/VL-G100C) between the VH and VL domain of the second Fv region in Construct 2 resulted in a significant reduced yield and monomer content when compared to corresponding Construct 1 lacking the stabilizing disulfide bridge.

TABLE 14
Yields and final monomer content.

| | | | Volu- | |
|---|---|---|---|---|
| | | | metric | Monomer |
| | | | Yield | Content |
| Construct | | SEQ ID NOs: | [mg/L] | [%] |
| Contruct 1 | Trastuzumab/I2C | 18, 19, 20 | 62* | 88* |
| Construct 2 | Trastuzumab/I2C | 21, 22, 23 | 29 | 65 |
| | (VH-G44C/VL- | | | |
| | G100C) | | | |
| Contruct 3 | Trastuzumab/Roche | 24, 25, 26 | 75 | 90 |
| Contruct 4 | Trastuzumab/SP34 | 27, 28, 29 | 53 | 90 |
| Contruct 5 | Trastuzumab/Neg. | 30, 31, 32 | 69 | 90 |
| | Ctrl. | | | |

*average of n = 2

### Example 2: Binding of Trivalent Bispecific Antigen-Binding Molecules to CD3 and HER2 Expressed on Cells

### Target Cells:

For the assessment of HER2 targeting of the bispecific trivalent Constructs 1, 3, 4 and 5 with bivalent binding to HER2 and monovalent binding to CD3, the following tumor cell lines were used: the HER2 positive human adenocarcinoma SKOV-3 [SKOV3] (ATCC® HTB-77™) cell line and the HER2 negative human adenocarcinoma MDA-MB-468 (ATCC® HTB-132™) cell line. In addition, a human CD3 positive T cell leukemia cell line, Jurkat (ATCC #TIB-152) was used to assess binding to human CD3.

### Method:

Jurkat or SKOV-3 cells were resuspended and counted in wash buffer (DPBS with calcium and magnesium (Gibco, #14040174) supplemented with 3% FBS and 0.02% sodium acid). 6E+04 cells per well were seeded in 384 well V-bottom plates (Greiner bio-one, #781280) and incubated with serially diluted constructs (titration range 500 nM to 0.5 nM) for 1 h on ice. Cells were washed 2 times in wash buffer. Bound constructs were detected using AlexaFluor647-conjugated detection antibody directed against human F(ab′)₂ fragment (Jackson Immuno Research, #109-606-097). Construct staining was measured using IntelliCyt iQue flow cytometer and analyzed in or ForeCyt (version 4.1.5379, IntelliCyt) software. EC₅₀ values were calculated using 4-parameter non-linear regression analysis in Prism software (GraphPad Software Inc., version 5.04).

### Results:

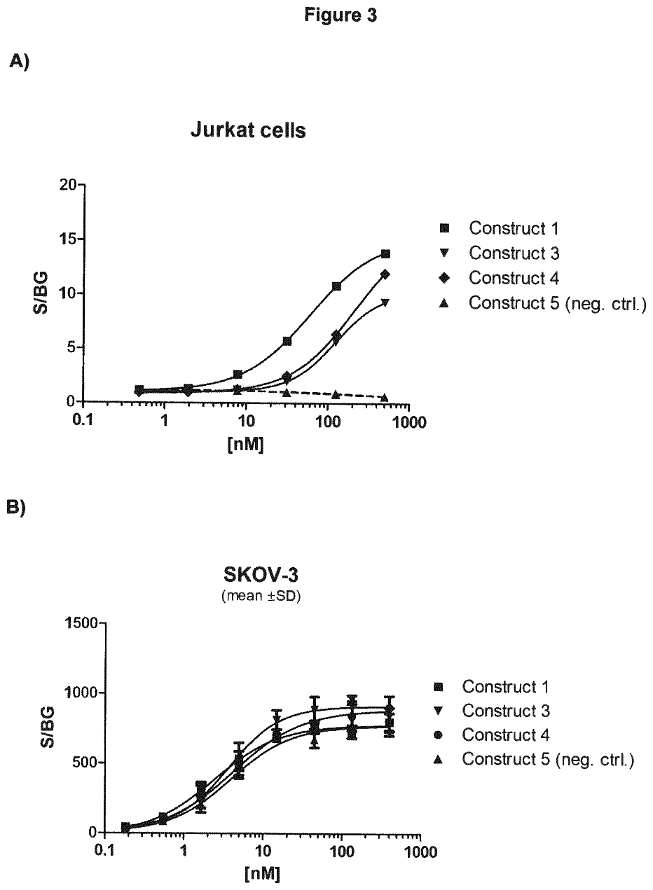

Results of the experiment are summarized in Table 15 and FIG. 3A (Jurkat cells) and FIG. 3B (SKOV-3 cells) and reveal that the bispecific trivalent Constructs 1, 3 and 4 specifically bind to HER2 and CD3 expressed on cells in a dose dependent manner. Furthermore, no binding to the HER2 and CD3 negative cell line is observable for the tested constructs (data not shown). Negative control Construct 5 shows no binding activity using either cell line.

TABLE 15
Cell binding of HER2 × CD3 bispecific
constructs to HER2 expressing SKOV-3 cells

| | | | FACS |
|---|---|---|---|
| | | | EC₅₀ [nM] |
| Construct | | SEQ ID NOs: | SKOV-3 |
| Construct 1 | Trastuzumab/I2C | 18, 19, 20 | 2.41 |
| Construct 3 | Trastuzumab/Roche | 24, 25, 26 | 3.72 |
| Construct 4 | Trastuzumab/SP34 | 27, 28, 29 | 4.17 |
| Construct 5 | Trastuzumab/Neg. Ctrl. | 30, 31, 32 | 3.96 |

### Example 3: Reporter Gene Assay—Testing of Trivalent Bispecific Antigen-Binding Molecules on SKOV-3 and Jurkat Cells Transfected with the NFAT Reporter Gene

### Target and Effector Cells:

For the evaluation of the functional activity of the bispecific trivalent Constructs 1, 3, 4 and 5, Jurkat cells (ATCC #TIB-152) transiently transfected with an NFAT reporter gene construct were used as surrogate effector cells. As target cells the following tumor cell lines were used: the HER2 positive human adenocarcinoma SKOV-3 [SKOV3] (ATCC® HTB-77™) cell line and the HER2 negative human adenocarcinoma MDA-MB-468 (ATCC® HTB-132™) cell line.

The following growth media were used for maintenance of the cell lines: (a) Jurkat: RPMI-1640+L-Glutamine (Thermo Fisher, #21875-034) supplemented with 10% FCS (Sigma, #F7524); (b) SKOV-3: McCoys 5a (ThermoFisher, #10938), supplemented with 10% FCS (Sigma #F7524) (c) MDA-MB-468: DMEM-L-Glutamine (ThermoFisher, #10938) supplemented with 1× GlutaMAX™ (ThermoFisher, #35050-061), 1× Sodium Pyruvate (ThermoFisher, #11360-039) and 10% FCS (Sigma #F7524).

### Method:

SKOV-3 and MDA-MB-468 cells were diluted in growth medium to a density of 4E+05 cells/ml. 100 μl cell suspension corresponding to 40,000 cells were seeded in each well of a tissue culture treated 96 well plate (Corning, #3917) and incubated overnight in a humidified incubator at 37° C. and 5% CO₂. Jurkat cells were resuspended in growth medium to a concentration of 2.5E+05 cells/ml. Transfection components pGL4.30[luc2P/N FAT-RE/Hygro] reporter gene vector, OptiMEM-I medium (Life Technologies, #31985-047) and TransIT-LT1 transfection reagent (Mirus, #MIR2304) were incubated for 15 min at RT, then added to the Jurkat cell suspension and incubated for 17 h in a humidified incubator at 37° C. and 5% CO₂. Jurkat cells were harvested and resuspended in growth medium at a concentration of 1.2E+06/ml. Medium was removed from coated target cells and replaced by 50 μl Jurkat cell suspension corresponding to 60,000 cells per well. Constructs 1, 3, 4 and negative control Construct 5 were serially diluted in Jurkat growth medium. 50 μl construct dilution was added to each well resulting in a final concentration range of 31 nM to 0.12 nM. Assay plates were incubated for 5 h in a humidified incubator at 37° C. and 5% CO₂. Bright-Glo™ Reagent (Promega, #E2620) was reconstituted according to manufacturer's instructions. Assay plates and reagent were equilibrated at room temperature. 100 μl of the Bright-Glo™ reagent was added to each well of the assay plate and mixed. Luminescence was measured using an InfiniteM1000 Pro plate reader (Tecan).

### Results:

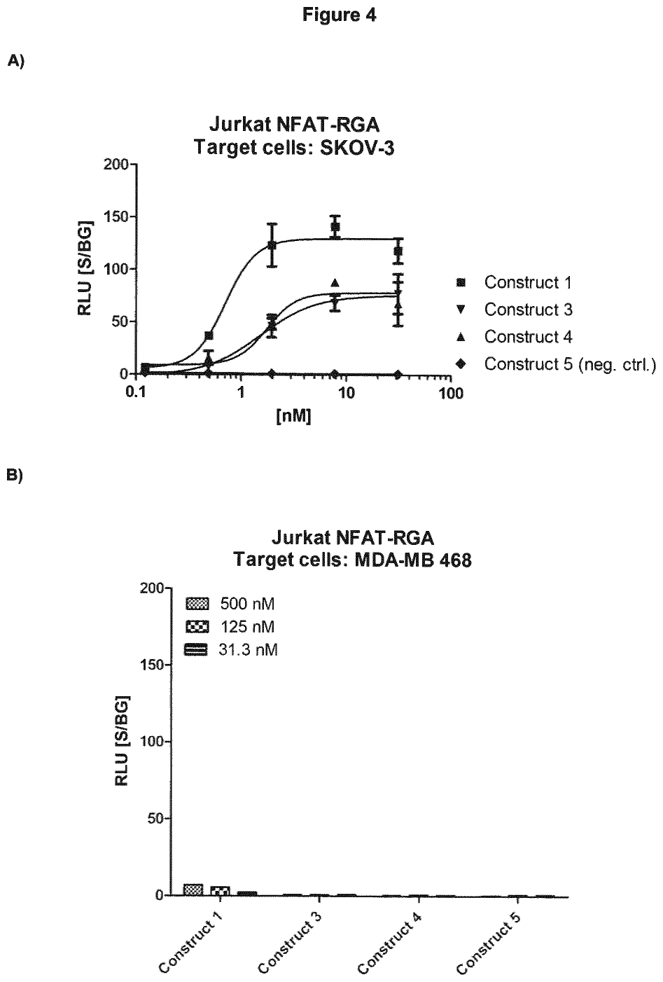

The results of the experiments are summarized in Table 16 and FIG. 4A (SKOV-3 cells) and FIG. 4B (MDA-MB-468 cells). Constructs 1, 3, 4 induce dose-dependent luciferase activity in the presence of the HER2 expressing target cell line SKOV-3 with EC₅₀ concentrations ranging from 0.7 nM to 1.8 nM. Construct 1 shows the highest efficacy and potency as indicated by EC₅₀ and maximum luciferase activity level, respectively. In the presence of HER2-negative MDA-MB-468 target cells, Construct 1 induces weak luciferase activity at high concentrations. Constructs 3, 4 are inactive in the absence of HER2. Negative control Construct 5 shows no activity using either target cell line.

TABLE 16
Induction of luciferase activity of HER2 × CD3
bispecific constructs in the presence of SKOV-3 cells.

| | | | EC₅₀ |
|---|---|---|---|
| | | | [nM] |
| Construct | | SEQ ID NOs: | SKOV-3 |
| Construct 1 | Trastuzumab/I2C | 18, 19, 20 | 0.71 |
| Construct 3 | Trastuzumab/Roche | 24, 25, 26 | 1.53 |
| Construct 4 | Trastuzumab/SP34 | 27, 28, 29 | 1.75 |
| Construct 5 | Trastuzumab/Neg. Ctrl. | 30, 31, 32 | no activity |

### Example 4: Re-Directed T-Cell Cytotoxicity Mediated by Trivalent Bispecific Antigen-Binding Molecules

Bispecific trivalent Constructs 1, 3, 4 and 5 were analyzed for their potential to induce T-cell-mediated killing of tumor cells upon binding to CD3 and HER2.

### Method

Human whole blood from healthy donors was collected in Li-Heparin containing S-Monovette containers (Sarstedt). Blood was transferred to 50 ml conical tubes and mixed with an equal volume of PBS containing 2% fetal bovine serum (Sigma, #F7524) and 2 mM EDTA. Diluted blood was transferred to SepMate-50 tubes (StemCell Technologies, #86450) containing 15 ml Biocoll solution (Biochrom, #L6115) and centrifuged for 10 min at 1200×g. Supernatant was transferred into a 50 ml conical tube, diluted to 45 ml with PBS and centrifuged for 8 min at 300×g. Supernatant was discarded, cell pellet resuspended in 1 ml PBS and cells counted using a Neubauer chamber.

5,000 HER2 expressing SKBR3 cells and HER2 negative MDA-MB-468 cells were suspended in culture medium (SKBR3: McCoy's 5A Medium (ThermoFisher, #26600), 10% FCS (Sigma, #F7524); MDA-MB-468: DMEM (ThermoFisher, #10938), 1× GlutaMAX™ (ThermoFisher, #35050-061), 1× Sodium Pyruvate (ThermoFisher, #11360-039), 10% FCS seeded in black 96 well assay plates (Corning, #3340) and incubated over night at 37° C. and 5% CO₂. CellToxGreen dye (Promega, #G8731), bispecific antigen-binding molecules diluted to 100 nM and 100,000 purified PBMCs, all diluted in assay medium comprising RPMI 1640 w/o Phenol red (Gibco, #32404-014), GlutaMAX and 10% fetal bovine serum, were added to the cells and incubated for 48 h at 37° C. and 5% CO₂. Cytotoxic activity was assessed by measuring incorporated CellToxGreen fluorescence at 485 nm excitation and 535 nm emission using a Tecan Infinite F500 device.

### Results:

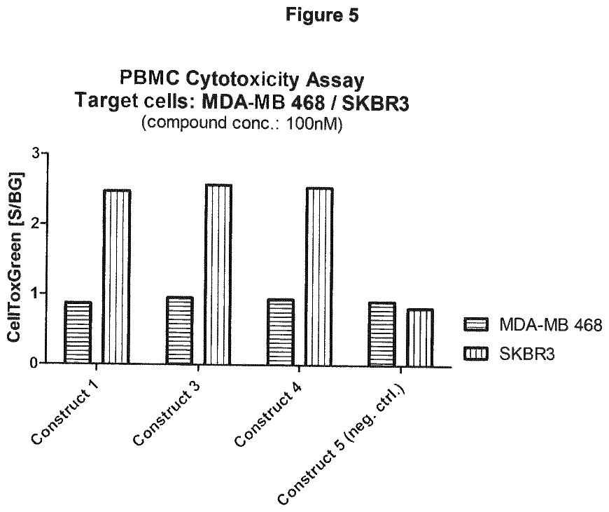

The results of the experiments are shown in FIG. 5. Co-cultivation of PBMCs with Construct 1, 3 and 4 induces killing of HER2 expressing SKBR3 target cells at a tested construct concentration of 100 nM. In the absence of the HER2 negative MDA-MB-468 cells, none of the tested constructs stimulates cytotoxic activity. Negative control Construct 5 induces no activity using either target cell line.

### Example 5: Affinity Determination

### Methods

Kinetic characterization of the interaction between human CD3 epsilon and an antigen-binding molecule with monovalent specificity for CD3 and bivalent specific for HER2 (Construct 1 and 3) was carried out in antigen-binding molecule capture format, with the antigen being applied as analyte in solution. High-capacity capture surfaces were prepared by loading biotinylated MabSelect SuRe ligand (non-biotinylated ligand: GE Healthcare, 28-4018-60) onto several streptavidin sensors (fortébio, part 18-5021). Each cycle of the kinetic experiment consisted of capture steps (of one ligand on several sensors used in parallel), followed by an analyte binding step (association phase, different analyte concentrations and assay buffer, i.e. antigen concentration 0 for blank subtraction). After binding, the dissociation of bound antigen was monitored (sensors exposed to assay buffer). At the end of each cycle, bound ligand and/or ligand-antigen complex was removed from the sensor surfaces by 2 consecutive regeneration steps à 20 s with 10 mM Glycine/HCl pH1.5 (GE Healthcare, BR 100354), while maintaining the integrity of the capture surface.

Signals recorded on the sensor with captured ligand, but exposed to assay buffer instead of antigen during binding were subtracted from the sensorgrams with non-zero antigen concentrations to correct e.g. potential dissociation of captured ligand. Association was recorded for 300 s and dissociation for 300 s at an orbital shaking speed of 1000 rpm. DPBS (GIBCO, no Ca²⁺, no Mg²⁺; Thermo Fisher Cat. No. 14190) supplemented with 0.05% (v/v) Polysorbate 20 (Merck, 8.22184.0500) and 0.1% (w/v) bovine serum albumin (Sigma, A7906) was used as assay buffer. Capture levels of ligands were adjusted to approx. 2 nm to achieve saturation levels R max of approx. 0.3 nm by the hCD3e analyte. Seven different analyte concentrations were used for analysis during kinetic experiments (pMAX_hCD3e(1-118)_F-chLys_avi; applied molarities 15.625-1000 nM, in a 2-fold serial dilution series).

Sensorgrams were evaluated with Data Analysis Software v 10 (Octet/fortébio). All sensorgrams were fitted to a 1:1 binding model to determine kₒₙ and k_{off} rate constants, which were used to calculate the K_{D} value. For kinetic profiles deviating from the expected 1:1 binding, the sensorgrams were evaluated using a best approximation to the monovalent kinetics, and results marked with comment “heterogeneous binding”. These results are considered less precise than kinetic profiles completely following the expected monovalent binding kinetics, but are assumed to be good approximations for K_{D}. Additionally, extrapolated saturation levels (R max) were set into relation with the obtained capture levels of ligand, taking into account the respective molecular weights of ligand and CD3 protein to assess if the observed binding events could be explained with the expected stoichiometry and monovalent binding.

### Results: Binding to Human CD3 Epsilon

The results of the experiments are summarized in Table 17. The observed binding was used to extrapolate the saturation level of CD3, and set into relation to the capture level of ligand. The experimental saturation R max was found within in the range (100±15%) theoretically expected for monovalent binding of CD3 to a fully active, monovalent antibody.

The results reveal that the relative position of the second Fv region with specificity for CD3 is not detrimental to the binding activity of the used CD3 specific antibody.

TABLE 17
Affinities of bispecific trivalent Construct 1 and Construct 3 with
monovalent binding to human CD3 epsilon

| | | SEQ ID | kon | koff | KD | |
|---|---|---|---|---|---|---|
| Construct | | NO: | [1/Ms] | [1/s] | [nM] | Comment |
| Construct 1 | Trastuzumab/ | 18, 19, | 4.57E+05 | 4.47E−04 | 1.0 | slightly heterogenous |
| | I2C | 20 | | | | binding |
| Construct 3 | Trastuzumab/ | 24, 25, | 5.19E+05 | 3.24E−03 | 6.2 | — |
| | Roche | 26 | | | | |

1. An antigen-binding molecule, comprising
a) a first Fab comprising a first Fv region, which specifically binds to a first antigen,
b) a second Fv region which specifically binds to a second antigen and
c) a second Fab comprising a third Fv region, which specifically binds to a third antigen, and
d) a Fc region composed of a first and second Fc region subunit; wherein
i. the C-terminus of the heavy or light chain of the first Fab is fused to the N-terminus of the VH or VL of the second Fv region, and wherein
ii. the C-terminus of the VH or VL of the second Fv region is fused to the N-terminus of the first Fc region subunit and the N-terminus of the second Fc subunit is fused to the C-terminus of the complementary variable domain of the second Fv region, and wherein
iii. the C-terminus of the heavy or light chain the second Fab is fused to the N-terminus of the VH or VL of the second Fv region with the proviso that the first and second Fab are fused to distinct variable domains of the second Fv region, and wherein
iv. in the CH3 domain of the first Fc region subunit, the threonine residue at position 366 is replaced with a tryptophan residue (T366W) and the serine residue at position 354 is replaced with a cysteine residue (S354C) and in the CH3 domain of the second Fc region subunit the tyrosine residue at position 407 is replaced with a valine residue (Y407V), the threonine residue at position 366 is replaced with a serine residue (T366S), the leucine residue at position 368 is replaced with an alanine residue (L368A) and the tyrosine residue at position 349 is replaced by a cysteine residue (Y349C) with numbering according EU index.

2. The antigen-binding molecule according to claim 1, wherein each fusion occurs via a peptide linker.

3. The antigen-binding molecule according to claim 1, wherein the antigen-binding molecule is composed of at least 4 polypeptides, wherein
a. a first polypeptide comprises the light or heavy chain of the first Fab,
b. a second polypeptide comprises from its N-terminus to its C-terminus
i. the complementary light or heavy chain of the first Fab,
ii. the VH or VL of the second Fv region and
iii. the first or second Fc region subunit
c. a third polypeptide comprises from its N-terminus to its C-terminus
i. the light or heavy chain of the second Fab,
ii. the complementary VH or VL of the second Fv region and
iii. the complementary first or second Fc region subunit
d. a fourth polypeptide comprises the complementary light or heavy chain of the second Fab.

4. The antigen-binding molecule according to claim 1, wherein the third antigen is identical to the first antigen.

5. The antigen-binding molecule according to claim 1, wherein the antigen-binding molecule provides bivalent binding to the first antigen and monovalent binding to the second antigen.

6. The antigen-binding molecule according to claim 1 wherein the antigen-binding molecule is a trivalent bispecific antigen-binding molecule.

7. The antigen-binding molecule according to claim 1, wherein the second antigen is expressed on an immune effector cell.

8. The antigen-binding molecule according to claim 1, wherein the first antigen is a member of the T-cell receptor complex.

9. The antigen-binding molecule according to claim 1, wherein the second antigen is CD3.

10. The antigen-binding molecule according to claim 1, wherein the Fc region is a human IgG1 Fc region.

11. The antigen-binding molecule according to claim 1, wherein the Fc region comprises one or more amino acid modification promoting the association of the first and second Fc region subunit.

12. The antigen-binding molecule according to claim 1, wherein in each of the Fc region subunit at least 5 amino acid residues in the positions corresponding to positions L234, L235, G237, A330, P331 with numbering according EU index in a human IgG1 are mutated to A, E, A, S, and S, respectively.

13. A pharmaceutical composition comprising the antigen-binding molecule according to claim 1 and a pharmaceutically acceptable carrier or excipient.

14. (canceled)

15. A method for re-directing cytotoxic activity of a T-cell to a cancer cell comprising contacting said cancer cell in the presence of a T-cell with a antigen-binding molecule according to claim 1.

16. A method for treating a disease in an individual, said method comprising administering an effective amount of the pharmaceutical composition of claim 13 to the individual.

17. The method of claim 16 wherein the disease is an autoimmune disease, an inflammatory disease, cancer, a vascular disease, an infectious disease, thrombosis, myocardial infarction or diabetes.

18. The method of claim 16 wherein the disease is a proliferative disease.

19. The method of claim 16 wherein the disease is cancer.


SEQUENCE LISTING (39 sequences)

SEQ ID NO: 1 - protein, 104 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 2 - protein, 125 aa, Artificial Sequence
  source: /note=“Description of Artificial Sequence Synthetic polypeptide”
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSS

SEQ ID NO: 3 - protein, 111 aa, Artificial Sequence
  source: /note=“Description of Artificial Sequence Synthetic polypeptide”
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGQ

SEQ ID NO: 4 - protein, 125 aa, Artificial Sequence
  source: /note=“Description of Artificial Sequence Synthetic polypeptide”
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 5 - protein, 109 aa, Artificial Sequence
  source: /note=“Description of Artificial Sequence Synthetic polypeptide”
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 6 - protein, 125 aa, Artificial Sequence
  source: /note=“Description of Artificial Sequence Synthetic polypeptide”
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 7 - protein, 111 aa, Artificial Sequence
  source: /note=“Description of Artificial Sequence Synthetic polypeptide”
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLGQ

SEQ ID NO: 8 - protein, 120 aa, Artificial Sequence
  source: /note=“Description of Artificial Sequence Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 9 - protein, 107 aa, Artificial Sequence [sequence omitted]

SEQ ID NO: 10 - protein, 9 aa, Artificial Sequence
  source: /note=“Description of Artificial Sequence Synthetic peptide”
GGSGGSGGS

SEQ ID NO: 11 - protein, 6 aa, Artificial Sequence
  source: /note=“Description of Artificial Sequence Synthetic peptide”
ASTKGP

SEQ ID NO: 12 - protein, 6 aa, Artificial Sequence
  source: /note=“Description of Artificial Sequence Synthetic peptide”
QPKAAP

SEQ ID NO: 13 - protein, 10 aa, Homo sapiens
DKTHTCPPCP

SEQ ID NO: 14 - protein, 16 aa, Artificial Sequence
  source: /note=“Description of Artificial Sequence Synthetic peptide”
ASTKGPDKTHTCPPCP

SEQ ID NO: 15 - protein, 16 aa, Artificial Sequence
  source: /note=“Description of Artificial Sequence Synthetic peptide”
QPKAAPDKTHTCPPCP

SEQ ID NO: 16 - protein, 227 aa, Artificial Sequence
  source: /note=“Description of Artificial Sequence Synthetic polypeptide”
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 227 aa, Artificial Sequence
  source: /note=“Description of Artificial Sequence Synthetic polypeptide”
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 18 - protein, 575 aa, Artificial Sequence
  source: /note=“Description of Artificial Sequence Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGSGGSGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGQPKAAPDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 590 aa, Artificial Sequence
  source: /note=“Description of Artificial Sequence Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGSGGSGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 20 - protein, 214 aa, Artificial Sequence
  source: /note=“Description of Artificial Sequence Synthetic polypeptide”
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 576 aa, Artificial Sequence
  source: /note=“Description of Artificial Sequence Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGSGGSGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGCGTKLTVLGQQPKAAPDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 590 aa, Artificial Sequence
  source: /note=“Description of Artificial Sequence Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGSGGSGGSEVQLVESGGGLVQPGGSLKLSCAASGFTFNKYAMNWVRQAPGKCLEWVARIRSKYNNYATYYADSVKDRFTISRDDSKNTAYLQMNNLKTEDTAVYYCVRHGNFGNSYISYWAYWGQGTLVTVSSASTKGPDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 23 - protein, 214 aa, Artificial Sequence; identical to SEQ ID NO: 20
  source: /note=“Description of Artificial Sequence Synthetic polypeptide”

SEQ ID NO: 24 - protein, 575 aa, Artificial Sequence
  source: /note=“Description of Artificial Sequence Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGSGGSGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLGQPKAAPDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 590 aa, Artificial Sequence
  source: /note=“Description of Artificial Sequence Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGSGGSGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASTKGPDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 26 - protein, 214 aa, Artificial Sequence; identical to SEQ ID NO: 20
  source: /note=“Description of Artificial Sequence Synthetic polypeptide”

SEQ ID NO: 27 - protein, 575 aa, Artificial Sequence
  source: /note=“Description of Artificial Sequence Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGSGGSGGSQAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGQPKAAPDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 590 aa, Artificial Sequence
  source: /note=“Description of Artificial Sequence Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGSGGSGGSEVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSSASTKGPDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 29 - protein, 214 aa, Artificial Sequence; identical to SEQ ID NO: 20
  source: /note=“Description of Artificial Sequence Synthetic polypeptide”

SEQ ID NO: 30 - protein, 573 aa, Artificial Sequence
  source: /note=“Description of Artificial Sequence Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGSGGSGGSDIELTQPPSVSVAPGQTARISCSGDNLPAYTVTWYQQKPGQAPVLVIYDDSDRPSGIPERFSGSNSGNTATLTISGTQAEDEADYYCASWDPSSGVVFGGGTKLTVLGQPKAAPDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 593 aa, Artificial Sequence
  source: /note=“Description of Artificial Sequence Synthetic polypeptide”
QVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGSGGSGGSQVQLQQSGPGLVKPSQTLSLTCAISGDSVSSNSAAWSWIRQSPGRGLEWLGRIYYRSKWYNDYAVSVKSRITINPDTSKNQFSLQLNSVTPEDTAVYYCARLDHRYHEDTVYPGMDVWGQGTLVTVSSASTKGPDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 32 - protein, 214 aa, Artificial Sequence; identical to SEQ ID NO: 20
  source: /note=“Description of Artificial Sequence Synthetic polypeptide”

SEQ ID NO: 33 - protein, 15 aa, Artificial Sequence
  source: /note=“Description of Artificial Sequence Synthetic peptide”
GGGGSGGGGSGGGGS

SEQ ID NO: 34 - protein, 20 aa, Artificial Sequence
  source: /note=“Description of Artificial Sequence Synthetic peptide”
  SITE (1)..(20): /note=“This sequence may encompass 1-10 ′Gly Ser′ repeating units”
GSGSGSGSGSGSGSGSGSGS

SEQ ID NO: 35 - protein, 50 aa, Artificial Sequence
  source: /note=“Description of Artificial Sequence Synthetic polypeptide”
  SITE (1)..(50): /note=“This sequence may encompass 1-10 ′Gly Gly Gly Gly Ser′ repeating units”
GGGGSGGGGSGGGGSGGGGSGGGGSGGGGSGGGGSGGGGSGGGGSGGGGS

SEQ ID NO: 36 - protein, 50 aa, Artificial Sequence
  source: /note=“Description of Artificial Sequence Synthetic polypeptide”
  SITE (1)..(50): /note=“This sequence may encompass 1-10 ′Ser Gly Gly Gly Gly′ repeating units”
SGGGGSGGGGSGGGGSGGGGSGGGGSGGGGSGGGGSGGGGSGGGGSGGGG

SEQ ID NO: 37 - protein, 50 aa, Artificial Sequence
  source: /note=“Description of Artificial Sequence Synthetic polypeptide”
  SITE (1)..(50): /note=“This sequence may encompass 1-10 ′Gly Ser Gly Gly Ser′ repeating units”
GSGGSGSGGSGSGGSGSGGSGSGGSGSGGSGSGGSGSGGSGSGGSGSGGS

SEQ ID NO: 38 - protein, 40 aa, Artificial Sequence
  source: /note=“Description of Artificial Sequence Synthetic polypeptide”
  SITE (1)..(40): /note=“This sequence may encompass 1-10 ′Gly Gly Gly Ser′ repeating units”
GGGSGGGSGGGSGGGSGGGSGGGSGGGSGGGSGGGSGGGS

SEQ ID NO: 39 - protein, 54 aa, Artificial Sequence
  source: /note=“Description of Artificial Sequence Synthetic polypeptide”
  SITE (5)..(54): /note=“This region may encompass 1-10 ′Ser Gly Gly Gly Gly′ repeating units”
GGGGSGGGGSGGGGSGGGGSGGGGSGGGGSGGGGSGGGGSGGGGSGGGGSGGGG